(19) United States
(12) Patent Application Publication
(10) Pub. No.: US 2016/0115146 A1
(43) Pub. Date: Apr. 28, 2016

(54) 3-CARBOXY SUBSTITUTED COUMARIN DERIVATIVES WITH A POTENTIAL UTILITY FOR THE TREATMENT OF CANCER DISEASES

(71) Applicants: UNIVERSITE CATHOLIQUE DE LOUVAIN, Louvain-la-Neuve (BE); KATHOLIEKE UNIVERSITEIT LEUVEN, Leuven (BE)

(72) Inventors: Nihid Draoui, Louvain-la-Neuve (BE); Olivier Feron, Louvain-la-Neuve (BE); Olivier Riant, Louvain-la-Neuve (BE); Pierre Sonveaux, Louvain-la-Neuve (BE); Olivier Schicke, Louvain-la-Neuve (BE); Antony Femandes, Louvain-la-Neuve (BE); Amuri Kilonda, Leuven (BE); Jean-Christophe Vanherck, Leuven (BE); Arnaud Marchand, Leuven (BE)

(21) Appl. No.: 14/894,076

(22) PCT Filed: Jun. 6, 2014
(86) PCT No.: PCT/EP2014/061921

§ 371 Date: Nov. 25, 2015

(30) Foreign Application Priority Data
Jun. 7, 2013 (GB) 1310195.1
Sep. 10, 2013 (GB) 1316100.5

(51) Int. Cl. C07D 311/08

(52) U.S. Cl. CPC C07D 311/08

(57) ABSTRACT

The present invention relates to novel compounds. The present invention also relates to the compounds for use as a medicine, more in particular for the prevention or treatment of cancer, more in particular cancers expressing MCT1 and/or MCT4. The present invention also relates to a method for the prevention or treatment of cancer in animals or humans by using the novel compounds. The present invention furthermore relates to pharmaceutical compositions or combination preparations of the novel compounds and to the compositions or preparations for use as a medicine, more preferably for the prevention or treatment of cancer. The present invention also relates to processes for the preparation of the compounds.

### FIELD OF THE INVENTION

The present invention relates to novel compounds. The present invention also relates to the compounds for use as a medicine, more in particular for the prevention or treatment of cancer, more in particular cancers expressing MCT1 and/or MCT4. The present invention also relates to a method for the prevention or treatment of cancer in animals or humans by using the novel compounds.

The present invention furthermore relates to pharmaceutical compositions or combination preparations of the novel compounds and to the compositions or preparations for use as a medicine, more preferably for the prevention or treatment of cancer. The present invention also relates to processes for the preparation of the compounds.

### BACKGROUND OF THE INVENTION

Cancer cells consume large amounts of glucose to survive and proliferate. The high turnover of the glycolytic pathway uncoupled from mitochondrial respiration accounts for the high concentration of lactate (up to 10-40 mM) in tumors, that is proposed to be associated with tumor invasiveness and poor patient outcomes. Lactate however does not merely represent a waste metabolite or a biomarker of tumor aggressiveness. It can indeed be captured by oxidative tumor cells and reconverted into pyruvate to be used in the TCA cycle.² A synergy actually takes place between glycolytic tumor cells exporting lactate, and oxygenated cells importing it to feed their metabolism. In addition, lactate shuttle has been reported to occur in tumors between glycolytic tumor cells and endothelial cells as well as between tumor-associated fibroblasts and oxidative tumor cells. The finding that in tumors lactate is an important fuel for proliferation and survival identified the blocking of the influx of lactate by tumor cells as an anti-cancer strategy. Since monocarboxylate transporters (MCT) represent the major path for inward and outward lactate fluxes in cells, inhibiting MCTs responsible for this lactate transport represents a viable anti-cancer strategy.

The family of MCT (also named SLC16 solute carrier) is composed of 14 members. Among them, only four isoforms (MCT1-4) have been documented to act as proton-linked transporters that can carry short chain monocarboxylates such as lactate and pyruvate across cell membranes. In cancer cells, MCT1 and MCT4 are the most widely expressed. MCT1 shows a better affinity for L-lactate (Km 3-6 mM) than MCT4 (Km 25-30 mM), but MCT4 has a higher turnover rate than MCT1. These differences are consistent with their respective roles in tumors. With a high affinity for lactate, MCT1 enables lactate entry into oxidative tumor cells whereas low affinity MCT4 is mainly expressed in glycolytic tumor cells and tumor-associated fibroblasts that export lactate. The complementarity between MCT1 and MCT4 to drive lactate shuttle(s) in tumors, represents an attractive target for new anticancer drugs. MCT1 blockade (and blockade of other MCTs enabling lactate influx in cancers) can indeed prevent oxygenated tumor cells to use lactate and therefore force them to consume glucose more avidly. Consequently, hypoxic tumor cells that are essentially dependent on glucose and have limited or no access to replacement fuels die from glucose deprivation.

α-cyano-4-hydroxycinnamate (CHC) was historically reported as the first MCT inhibitor. CHC, however, is usually described to be active in the upper μM range and lacks specificity since in some experimental setups, it can also inhibit the mitochondrial pyruvate carrier. More recently, AR-C155858, a highly potent MCT1/MCT2 inhibitor was disclosed by Astra-Zeneca. This compound was demonstrated to strongly and selectively block MCT1 and MCT2 activity in activated T-lymphocytes, obstructing lactate efflux and thereby acting as a powerful immunosuppressive drug. The therapeutic effects of this compound in certain tumors is limited by the compensatory effects of MCT4 that can take the lead to facilitate lactate efflux when the high affinity MCT1 transporter is blocked.

Because of the natural organization of tumors in hypoxic and oxygenated compartments and since angiogenesis and fibroblast recruitment in the tumor microenvironment are common characteristics of cancers, virtually all cancer patients may benefit from the administration of inhibitors of lactate influx. We and other have reported the expression of MCT1 in a variety of human cancers including colon, breast, head and neck and lung cancers (Sonveaux et al., J Clin Invest. 2008 December; 118(12):3930-42) and central nervous system (glioma), gynecologic tract (incl. cervix), prostate, and stomach (Pinheiro et al., J Bioenerg Biomembr. 2012 February; 44(1):127-39).

There is still a great need for novel, alternative or better therapeutics for the prevention or treatment of cancers. Therapeutics with good potency, having less side-effects, a higher activity, a lower toxicity or better pharmacokinetic or -dynamic properties would be very welcome. The present invention provides a novel class of novel compounds which can be used in the prevention or treatment of cancer.

The present invention provides a class of novel compounds which can be used as inhibitors of lactate uptake and for the prevention or treatment of cancer or tumor formation.

### SUMMARY OF THE INVENTION

The present invention is based on the unexpected finding that at least one of the above-mentioned problems can be solved by a novel class of compounds.

The present invention provides new compounds which have been shown to possess lactate influx inhibitory activity. The present invention furthermore demonstrates that these compounds efficiently inhibit cancer cell proliferation when the cancer cells use lactate as energy source. Therefore, these compounds constitute a useful class of new potent compounds that can be used in the treatment and/or prevention of cancer in animals, mammals and humans, more particularly for the treatment and/or prevention of solid cancers or more in particularly, cancers expressing MCT1 and/or MCT4.

The new compounds are compounds of formula (A),

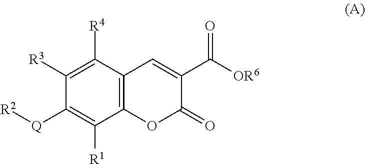

wherein,
each R¹, R³ and R⁴ is independently selected from hydrogen; halogen; hydroxyl; sulfhydryl; trifluoromethyl; trifluoromethoxy; nitro; amino; cyano; alkyl; alkenyl; alkynyl; heteroalkyl; heteroalkenyl; and heteroalkynyl;
   wherein said alkyl, alkenyl, alkynyl, heteroalkyl, heteroalkenyl, and heteroalkynyl can be unsubstituted or substituted with one or more substituents selected from R⁷; or
      R¹ or R³ can be combined with R² to form a 5, 6, or 7 membered heterocycle; or wherein R¹ or R³ can be combined with R⁵ to form a 5, 6, or 7 membered heterocycle;
Q is independently selected from 0 and NR⁵;
R² is independently selected from alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
      R² is taken together with R⁵ to form a 5, 6, or 7 membered heterocycle which can be unsubstituted or substituted with one or more substituents selected from R⁸;
R⁵ is independently selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
      R⁵ is taken together with R² to form a 4, 5, 6, or 7 membered heterocycle which can be unsubstituted or substituted with one or more substituents selected from R⁸;
R⁶ is independently selected from hydrogen; alkyl; and arylalkyl;
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
   and isomers (in particular stereo-isomers or tautomers), solvates, salts (in particular pharmaceutically acceptable salts) or prodrugs thereof.

The present invention furthermore relates to such compounds for use as medicines and to the use of the compounds for the manufacture of medicaments for treating and/or preventing cancer in subjects. The present invention furthermore relates to such compounds for use as medicines for the prevention or treatment of cancer, tumor formation and/or tumor relapse in subjects (animals, mammals, more in particular humans). The invention also relates to methods for the preparation of all such compounds and to pharmaceutical compositions comprising them in an effective amount.

The present invention also relates to a method of treatment or prevention of cancer in humans by the administration of one or more such compounds, optionally in combination with one or more other medicines, to a subject or a patient in need thereof. The present invention also relates to a method of treatment or prevention of tumor formation or tumor relapse in humans by the administration of one or more such compounds, optionally in combination with one or more other medicines, to a subject or a patient in need thereof.

The present invention also relates to combination preparations comprising a compound of the invention and another chemotherapeutic agents, more in particular cisplatin.

### DETAILED DESCRIPTION OF THE FIGURES

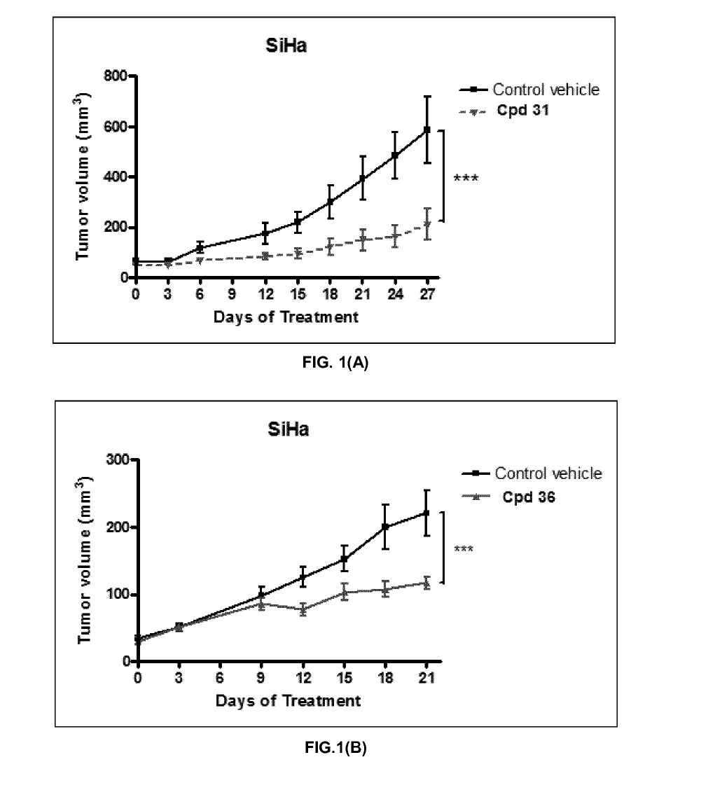

FIG. 1: Results of the evaluation of the anti-cancer activity of two compounds of the invention against cervix cancer (SiHa cells xenograft model). (A) cpd 31 (tested by using nude NMRI mice) (B) cpd 36 (tested by using nude Balb-c mice)

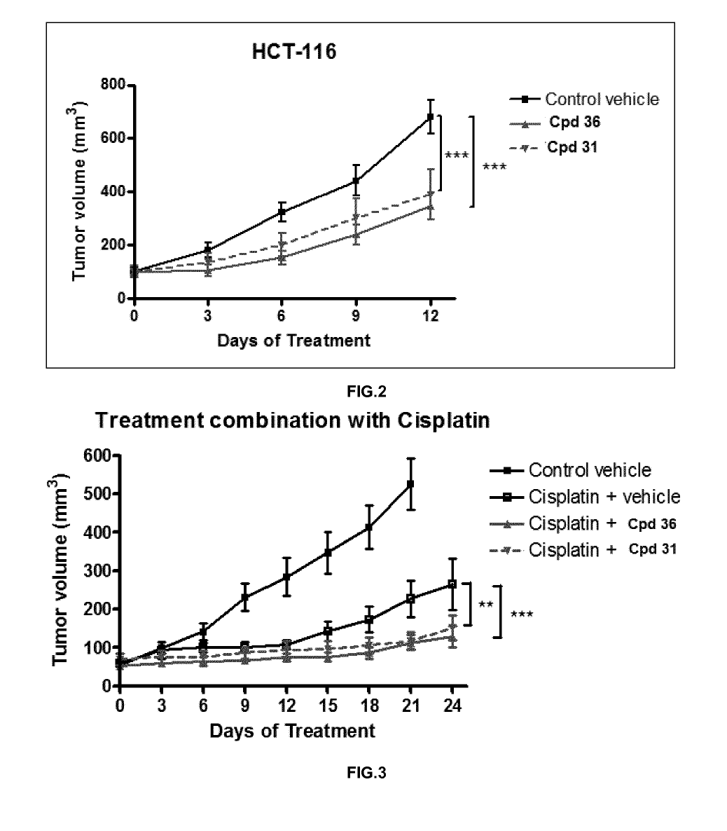

FIG. 2: Results of the evaluation of the anti-cancer activity of two compounds of the invention (cpd 31 and cpd 36) against colon cancer (HCT116 cells xenograft model).

FIG. 3: Results of the evaluation of the anti-cancer activity of two compounds of the invention (cpd 31 and cpd 36) against cervix cancer (SiHa cells xenograft model) in combination with standard chemotherapy, cisplatin.

### DETAILED DESCRIPTION OF THE INVENTION

The present invention will be described with respect to particular embodiments but the invention is not limited thereto.

One aspect of the present invention is the provision of new compounds, namely compounds of formula (A) as described in the summary of the invention.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous. Hereto, the present invention is in particular captured by any one or any combination of one or more of the below numbered aspects and embodiments 1 to 77, with any other statement and/or embodiments.

In a particular embodiment of all aspects, claims, statements or formulas of the invention, R² and R⁵ are not equal to each other or are not the same chemical group or atom. Alternatively worded, in this particular embodiment, R² and R⁵ have a different structure or R² and R⁵ are different from each other.
1. A compound according to formula (A) for use as a medicine,

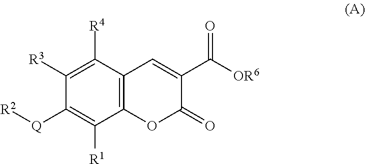

wherein,
each R¹, R³ and R⁴ is independently selected from hydrogen; halogen; hydroxyl; sulfhydryl; trifluoromethyl; trifluoromethoxy; nitro; amino; cyano; alkyl; alkenyl; alkynyl; heteroalkyl; heteroalkenyl; and heteroalkynyl;
   wherein said alkyl, alkenyl, alkynyl, heteroalkyl, heteroalkenyl, and heteroalkynyl can be unsubstituted or substituted with one or more substituents selected from R⁷; or
      R¹ or R³ can be combined with R² to form a 5, 6, or 7 membered heterocycle; or wherein R¹ or R³ can be combined with R⁵ to form a 5, 6, or 7 membered heterocycle;
Q is independently selected from 0 and NR⁵;
R² is independently selected from alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
      R² is taken together with R⁵ to form a 5, 6, or 7 membered heterocycle which can be unsubstituted or substituted with one or more substituents selected from R⁸;
R⁵ is independently selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
      R⁵ is taken together with R² to form a 4, 5, 6, or 7 membered heterocycle which can be unsubstituted or substituted with one or more substituents selected from R⁸;
R⁶ is independently selected from hydrogen; and alkyl;
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
   and isomers (in particular stereo-isomers or tautomers), solvates, salts (in particular pharmaceutically acceptable salts) or prodrugs thereof.
2. The compounds of statement 1, wherein R⁶ is hydrogen.
3. The compounds according to any one of statements 1 or 2, wherein R¹, R³ and R⁴ are hydrogen.
4. The compounds according to any one of statements 1 to 3, wherein R⁵ is selected from hydrogen; C₁₋₉ alkyl; and C₃₋₉ cycloalkyl, more in particular is selected from hydrogen and C₁₋₉ alkyl.
5. The compounds according to any one of statements 1 to 4, wherein R⁵ is selected from hydrogen; and C₁₋₉ alkyl.
6. The compounds according to any one of statements 1 to 5, wherein R² and R⁵ are not equal to each other or are not the same chemical group or atom. Alternatively worded, in this particular embodiment, R² and R⁵ have a different structure or R² and R⁵ are different from each other.
7. A compound according to formula (E1), (E2),(E3) or (E4) and isomers (in particular stereo-isomers or tautomers), solvates, salts (in particular pharmaceutically acceptable salts) or prodrugs thereof,

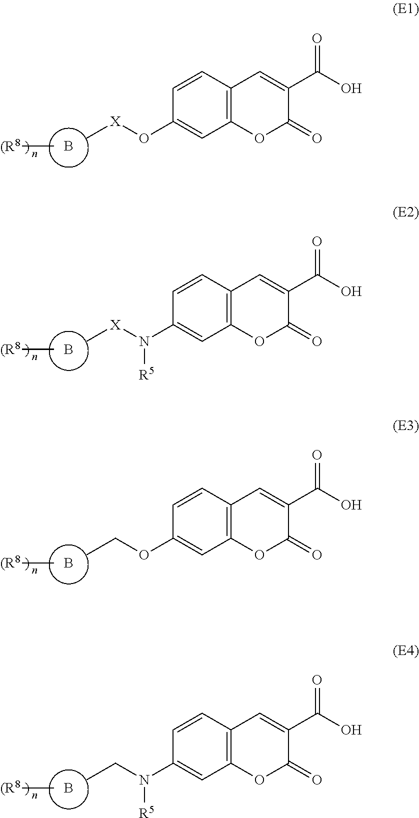

wherein,
each R⁵ and R⁸ is as in statements 1 to 6;
n is selected from 0; 1; 2; 3 and 4;
X is selected from alkylene or represents a single bond (thereby establishing a direct bond between 0 and cycle B for formula (E1) or N and cycle B for formula (E2)); and
cycle B is selected from cycloalkyl; cycloalkenyl; cycloalkynyl; aryl; and heterocycle.
8. A compounds of formula (A),

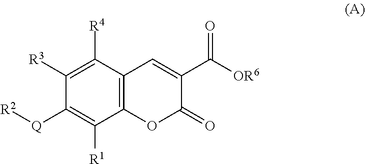

wherein,
each R¹, R³ and R⁴ is independently selected from hydrogen; halogen; hydroxyl; sulfhydryl; trifluoromethyl; trifluoromethoxy; nitro; amino; cyano; alkyl; alkenyl; alkynyl; heteroalkyl; heteroalkenyl; and heteroalkynyl;
   wherein said alkyl, alkenyl, alkynyl, heteroalkyl, heteroalkenyl, and heteroalkynyl can be unsubstituted or substituted with one or more substituents selected from R⁷; or
      R¹ or R³ can be combined with R² to form a 5, 6, or 7 membered heterocycle; or wherein R¹ or R³ can be combined with R⁵ to form a 5, 6, or 7 membered heterocycle;
Q is independently selected from 0 and NR⁵;
R² is independently selected from alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
      R² is taken together with R⁵ to form a 5, 6, or 7 membered heterocycle which can be unsubstituted or substituted with one or more substituents selected from R⁸;
R⁵ is independently selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
      R⁵ is taken together with R² to form a 4, 5, 6, or 7 membered heterocycle which can be unsubstituted or substituted with one or more substituents selected from R⁸;
R⁶ is independently selected from hydrogen; alkyl; and arylalkyl;
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
   and isomers (in particular stereo-isomers or tautomers), solvates, salts (in particular pharmaceutically acceptable salts) or prodrugs thereof.
9. The compound according to any one of statements 1 to 8, wherein said compound is a compound of formula (B),

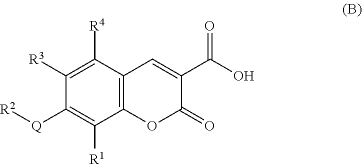

wherein, each of R¹, R³, R⁴, Q and R² have the same meaning as that defined in any one of statements 1 to 6.
10. The compound according to any one of statements 1 to 9, wherein said compound is a compound of formula (C),

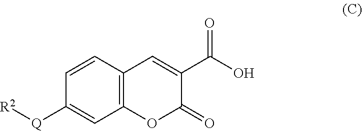

wherein, each of Q and R² have the same meaning as that defined in any one of statements 1 to 9.
11. The compound according to any one of statements 1 to 10, wherein said compound is a compound of formula (D1) or (D2),

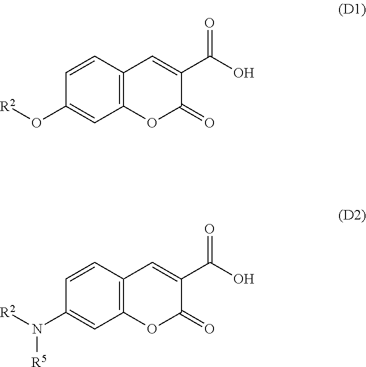

wherein, each of R² and R⁵ have the same meaning as that defined in any one of statements 1 to 10.
12. The compound according to any one of statements 1 to 11, wherein said compound is a compound of formula (E1), (E2), (E3) or (E4),

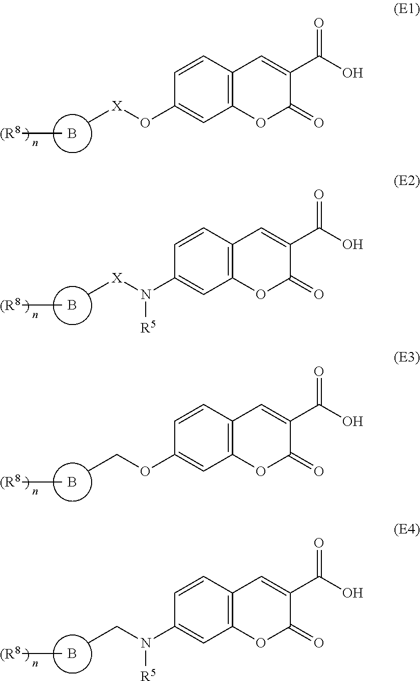

wherein,
R⁵ is selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹;
each R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
each R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
n is selected from 0; 1; 2; 3 and 4;
X is selected from alkylene, —C(═O)—, —SO₂—; or represents a single bond (thereby establishing a direct bond between 0 and cycle B for formula (E1) or N and cycle B for formula (E2)); and
cycle B is selected from cycloalkyl; cycloalkenyl; cycloalkynyl; aryl; and heterocycle.
13. The compound according to any one of statements 1 to 12, wherein said compound is a compound of formula (E5) or (E6),

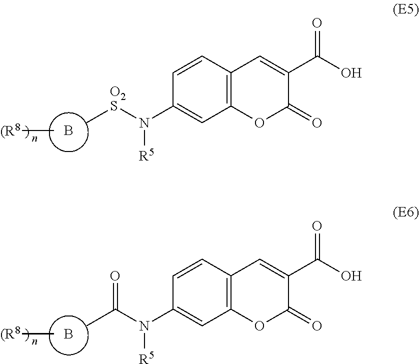

wherein, each of R⁹ and R⁵ have the same meaning as that defined in any one of statements 1 to 12,
n is selected from 0; 1; 2; 3 and 4; and
cycle B is selected from cycloalkyl; cycloalkenyl; cycloalkynyl; aryl; and heterocycle.
14. The compound according to any one of statements 1 to 13, wherein said compound is a compound of formula (F1) or (F2),

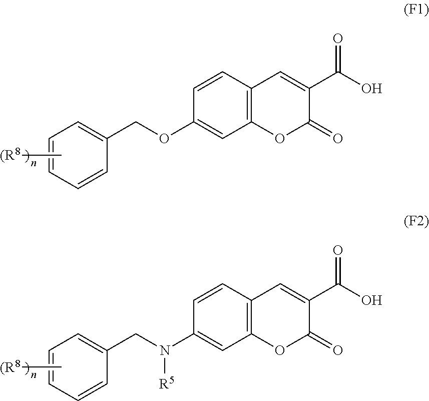

R⁵ is selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹;
each R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
each R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂; and
   n is selected from 0; 1; 2; 3 and 4.
15. The compound according to any one of statements 1 to 13, wherein said compound is a compound of formula (F3) or (F4),

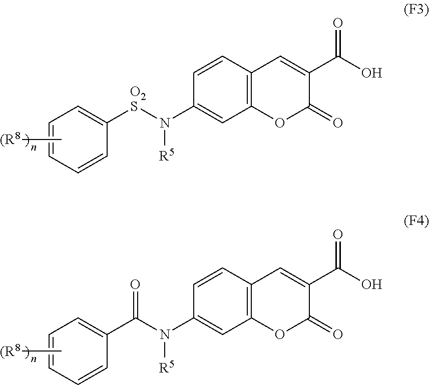

wherein, each of R⁸ and R⁵ have the same meaning as that defined in any one of statements 1 to 14, and
n is selected from 0; 1; 2; 3 and 4.
16. A compound according to formula (A) for use in the prevention and/or treatment of cancer in a subject,

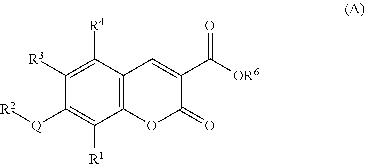

wherein,
each R¹, R³ and R⁴ is independently selected from hydrogen; halogen; hydroxyl; sulfhydryl; trifluoromethyl; trifluoromethoxy; nitro; amino; cyano; alkyl; alkenyl; alkynyl; heteroalkyl; heteroalkenyl; and heteroalkynyl;
   wherein said alkyl, alkenyl, alkynyl, heteroalkyl, heteroalkenyl, and heteroalkynyl can be unsubstituted or substituted with one or more substituents selected from R⁷; or
      R¹ can be combined with R² to form a 6, or 7 membered heterocycle; or R³ can be combined with R² to form a 5, 6, or 7 membered heterocycle; or wherein R¹ or R³ can be combined with R⁵ to form a 5, 6, or 7 membered heterocycle;
Q is independently selected from 0 and NR⁵;
R² is independently selected from alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
      R² is taken together with R⁵ to form a 5, 6, or 7 membered heterocycle which can be unsubstituted or substituted with one or more substituents selected from R⁸;
R⁵ is independently selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
      R⁵ is taken together with R² to form a 4, 5, 6, or 7 membered heterocycle which can be unsubstituted or substituted with one or more substituents selected from R⁸;
R⁶ is independently selected from hydrogen; and alkyl;
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
   and isomers (in particular stereo-isomers or tautomers), solvates, salts (in particular pharmaceutically acceptable salts) or prodrugs thereof.
17. A compound according to formula (A) for use in the prevention and/or treatment of cancer in a subject,

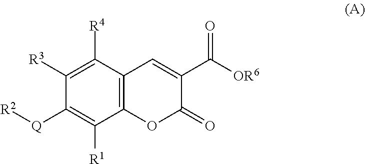

wherein,
each R¹, R³ and R⁴ is independently selected from hydrogen; halogen; hydroxyl; sulfhydryl; trifluoromethyl; trifluoromethoxy; nitro; amino; cyano; alkyl; alkenyl; alkynyl; heteroalkyl; heteroalkenyl; and heteroalkynyl;
   wherein said alkyl, alkenyl, alkynyl, heteroalkyl, heteroalkenyl, and heteroalkynyl can be unsubstituted or substituted with one or more substituents selected from R⁷; or
      R¹ can be combined with R² to form a 5, 6, or 7 membered heterocycle; and/or R³ can be combined with R² to form a 5, 6, or 7 membered heterocycle; or wherein R¹ and/or R³ can be combined with R⁵ to form a 5, 6, or 7 membered heterocycle;
Q is independently selected from 0 and NR⁵;
R² is independently selected from alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
      R² is taken together with R⁵ to form a 5, 6, or 7 membered heterocycle which can be unsubstituted or substituted with one or more substituents selected from R⁸;
R⁵ is independently selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
      R⁵ is taken together with R² to form a 4, 5, 6, or 7 membered heterocycle which can be unsubstituted or substituted with one or more substituents selected from R⁸;
R⁶ is independently selected from hydrogen; and alkyl;
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
   and isomers (in particular stereo-isomers or tautomers), solvates, salts (in particular pharmaceutically acceptable salts) or prodrugs thereof.
18. The compounds of statement 16 or 17, wherein R⁶ is hydrogen.
19. The compounds according to any one of statements 16 to 18, wherein R¹, R³ and R⁴ are hydrogen.
20. The compounds according to any one of statements 16 to 19, wherein R⁵ is selected from hydrogen; C₁₋₉ alkyl; and C₃₋₉ cycloalkyl, more in particular is selected from hydrogen and C₁₋₉ alkyl.
21. A compound of formula (D2) and isomers (in particular stereo-isomers or tautomers), solvates, salts (in particular pharmaceutically acceptable salts) or prodrugs thereof,

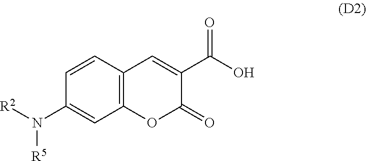

wherein,
R² is independently selected from cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, aryl heteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
R⁵ is independently selected from alkyl; cycloalkyl; alkenyl; cycloalkenyl; alkynyl; cycloalkynyl; heteroalkyl; heteroalkenyl; heteroalkynyl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
   and wherein said alkyl, cycloalkyl, alkenyl, cycloalkenyl, alkynyl, cycloalkynyl, heteroalkyl, heteroalkenyl, heteroalkynyl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
   and isomers (in particular stereo-isomers or tautomers), solvates, salts (in particular pharmaceutically acceptable salts) or prodrugs thereof.
22. The compounds according to statement 21, wherein R² and R⁵ are not equal to each other or are not the same chemical group or atom. Alternatively worded, in this particular embodiment, R² and R⁵ have a different structure or R² and R⁵ are different from each other.
23. The compound according to statement 21 or 22, wherein said compound is a compound of formula (E2), or (E4) and isomers (in particular stereo-isomers or tautomers), solvates, salts (in particular pharmaceutically acceptable salts) or prodrugs thereof,

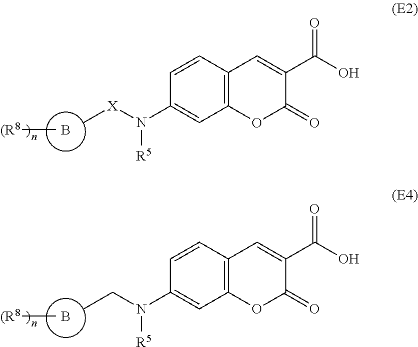

wherein,
each R⁵ and R⁹ is as in statement 21 or 22;
n is selected from 0; 1; 2; 3 and 4;
X is selected from alkylene, —CO—, —SO₂—, or represents a single bond (thereby establishing a direct bond between 0 and cycle B for formula (E1) or N and cycle B for formula (E2)); and
cycle B is selected from cycloalkyl; cycloalkenyl; cycloalkynyl; aryl; and heterocycle.
24. The compound according to any one of statements 21 to 23, wherein said compound is a compound of formula (E5) or (E6),

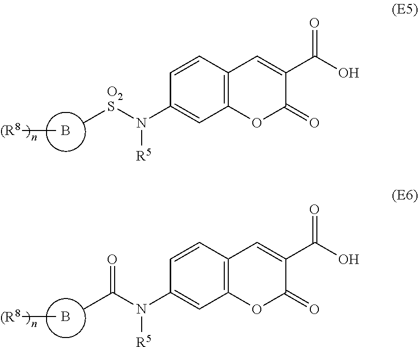

wherein, each of R⁸ and R⁵ have the same meaning as that defined in any one of statements 21 to 23,
n is selected from 0; 1; 2; 3 and 4; and
cycle B is selected from cycloalkyl; cycloalkenyl; cycloalkynyl; aryl; and heterocycle.
25. The compound according to any one of statements 21 to 24, wherein said compound is a compound of formula (F3) or (F4),

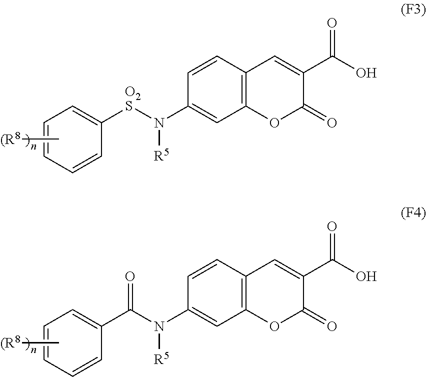

wherein, each of R⁸ and R⁵ have the same meaning as that defined in any one of statements 21 to 24, and
n is selected from 0; 1; 2; 3 and 4.
26. The compound according to statement 23, wherein each R⁵ and R⁸ is as in statement 21;
   n is selected from 0; 1; 2; 3 and 4;
   X is selected from alkylene, or represents a single bond (thereby establishing a direct bond between O and cycle B for formula (E1) or N and cycle B for formula (E2)); and cycle B is selected from cycloalkyl; cycloalkenyl; cycloalkynyl; aryl; and heterocycle.
27. The compounds according to any one of statements 1 to 26, for use as in the prevention and/or treatment of cancer in a subject.
28. The compounds according to statement 27, wherein the cancer is a solid cancer.
29. The compounds according to statement 27, wherein the cancer is a cancer expressing MCT1 and/or MCT4.
30. The compounds according to statement 29, wherein the cancer is a solid cancer expressing MCT1 and/or MCT4.
31. The compounds according to any one of statements 27 to 30, wherein the cancer is selected from cervix cancer and colon cancer.
32. A pharmaceutical composition comprising the compounds according to any one of statements 1 to 26 in combination with a pharmaceutically acceptable carrier.
33. A method for the prevention or treatment of a cancer in an animal, mammal or human comprising administering to said animal, mammal or human in need for such prevention or treatment an effective dose of the compounds according to any one of statements 1 to 26.
34. The compounds according to any one of statements 1 to 31, wherein each R¹, R³ and R⁴ is independently selected from hydrogen; halogen; trifluoromethyl; trifluoromethoxy; cyano; alkyl; and heteroalkyl; wherein said alkyl, and heteroalkyl can be unsubstituted or substituted with one or more substituents selected from R⁷.
35. The compounds according to any one of statements 1 to 31, 34, wherein each R¹, R³ and R⁴ is independently selected from hydrogen; halogen; trifluoromethyl; trifluoromethoxy; cyano; C₁₋₆ alkyl; and C₁₋₆ heteroalkyl; wherein said C₁₋₆ alkyl; and C₁₋₆ heteroalkyl can be unsubstituted or substituted with one or more substituents selected from R⁷.
36. The compounds according to any one of statements 1 to 31, 34 to 35, wherein each R¹, R³ and R⁴ is independently selected from hydrogen; halogen; trifluoromethyl; trifluoromethoxy; cyano; C₁₋₄ alkyl; and C₁₋₄ heteroalkyl.
37. The compounds according to any one of statements 1 to 31, 34 to 36, wherein each R¹, R³ and R⁴ is independently selected from hydrogen; F; Cl; trifluoromethyl; trifluoromethoxy; cyano; methyl; ethyl; propyl; butyl; methoxy; ethoxy; propyloxy; and dimethylaminomethylene.
38. The compounds according to any one of statements 1 to 31, 34 to 37, wherein each R¹, R³ and R⁴ is independently selected from hydrogen; F; Cl; trifluoromethyl; trifluoromethoxy; cyano; methyl; ethyl; methoxy; and ethoxy.
39. The compounds according to any one of statements 1 to 31, 34 to 37, wherein R² is independently selected from C₁₋₉ alkyl; C₃₋₉ cycloalkyl; C₂₋₉ alkenyl; C₃₋₉ cycloalkenyl; C₂₋₉ alkynyl; C₃₋₉ cycloalkynyl; C₁₋₉ heteroalkyl; C₂₋₉ heteroalkenyl; C₂₋₉ heteroalkynyl; aryl; heterocycle; aryl C₁₋₉ alkyl; aryl C₂₋₉ alkenyl; aryl C₂₋₉ alkynyl; aryl C₁₋₉ heteroalkyl; aryl C₂₋₉ heteroalkenyl; aryl C₂₋₉ heteroalkynyl; heterocycle-C₁₋₉ alkyl; heterocycle-C₂₋₉ alkenyl; heterocycle-C₂₋₉ alkynyl; heterocycle-C₁₋₉ heteroalkyl; heterocycle-C₂₋₉ heteroalkenyl; or heterocycle-C₂₋₉ heteroalkynyl;
   and wherein said C₁₋₉ alkyl, C₃₋₉ cycloalkyl, C₂₋₉ alkenyl, C₃₋₉ cycloalkenyl, C₂₋₉ alkynyl, C₃₋₉ cycloalkynyl, C₁₋₉ heteroalkyl, C₂₋₉ heteroalkenyl, C₂₋₉ heteroalkynyl, aryl, heterocycle, aryl C₁₋₉ alkyl, aryl C₂₋₉ alkenyl, aryl C₂₋₉ alkynyl, aryl C₁₋₉ heteroalkyl, aryl C₂₋₉ heteroalkenyl, aryl C₂₋₉ heteroalkynyl, heterocycle-C₁₋₉ alkyl, heterocycle-C₂₋₉ alkenyl, heterocycle-C₂₋₉ alkynyl, heterocycle-C₁₋₉ heteroalkyl, heterocycle-C₂₋₉ heteroalkenyl, or heterocycle-C₂₋₉ heteroalkynyl can be unsubstituted or substituted with one or more substituents selected from R⁸
40. The compounds according to any one of statements 1 to 31, 34 to 39, wherein R² is taken together with R⁵ to form a 5, 6, or 7 membered heterocycle which can be unsubstituted or substituted with one or more substituents selected from R⁸.
41. The compounds according to any one of statements 1 to 31, 34 to 40, wherein R² is independently selected from C₁₋₉ alkyl; C₃₋₉ cycloalkyl; C₁₋₉ heteroalkyl; aryl; heterocycle; aryl C₁₋₉ alkyl; aryl C₁₋₉ heteroalkyl; heterocycle-C₁₋₉ alkyl; heterocycle-C₁₋₉ heteroalkyl;
   and wherein said C₁₋₉ alkyl, C₃₋₉ cycloalkyl, C₁₋₉ heteroalkyl, aryl, heterocycle, aryl C₁₋₉ alkyl, aryl C₁₋₉ heteroalkyl, heterocycle-C₁₋₉ alkyl and heterocycle-C₁₋₉ heteroalkyl, can be unsubstituted or substituted with one or more substituents selected from R⁸; or R² is taken together with R⁵ to form a 5, 6, or 7 membered heterocycle which can be unsubstituted or substituted with one or more substituents selected from R⁸.
42. The compounds according to any one of statements 1 to 31, 34 to 41, wherein R² is independently selected from C₁₋₉ alkyl; C₃₋₉ cycloalkyl; C₁₋₉ heteroalkyl; aryl; heterocycle; aryl C₁₋₉ alkyl; heterocycle-C₁₋₉ alkyl;
   and wherein said C₁₋₉ alkyl, C₃₋₉ cycloalkyl, C₁₋₉ heteroalkyl, aryl, heterocycle, aryl C₁₋₉ alkyl, and heterocycle-C₁₋₉ alkyl, can be unsubstituted or substituted with one or more substituents selected from R⁸; or R² is taken together with R⁵ to form a 5, 6, or 7 membered heterocycle which can be unsubstituted or substituted with one or more substituents selected from R⁸.
43. The compounds according to any one of statements 1 to 31, 34 to 42, wherein R² is independently selected from aryl C₁₋₉ alkyl; aryl C₁₋₉ heteroalkyl, heterocycle-C₁₋₉ alkyl and heterocycle-C₁₋₉ heteroalkyl; wherein said aryl C₁₋₉ alkyl, aryl C₁₋₉ heteroalkyl, heterocycle-C₁₋₉ alkyl and heterocycle-C₁₋₉ heteroalkyl, can be unsubstituted or substituted with one or more substituents selected from R⁸; or R² is taken together with R⁵ to form a 5, 6, or 7 membered heterocycle which can be unsubstituted or substituted with one or more substituents selected from R⁸.
44. The compounds according to any one of statements 1 to 31, 34 to 43, wherein R² is independently selected from aryl C₁₋₉ alkyl and heterocycle-C₁₋₉ alkyl; wherein said aryl C₁₋₉ alkyl and heterocycle-C₁₋₉ alkyl, can be unsubstituted or substituted with one or more substituents selected from R⁸; or R² is taken together with R⁵ to form a 5, 6, or 7 membered heterocycle ring which can be unsubstituted or substituted with one or more substituents selected from R⁸.
45. The compounds according to any one of statements 1 to 31, 34 to 44, wherein R² is independently selected from arylmethyl-; arylethyl-; arylpropyl-; arylcarbonyl-; arylsulfonyl-; heterocyclemethyl-; heterocycle-ethyl-; heterocyclepropyl-; heterocyclecarbonyl-; and heterocyclesulfonyl-; wherein said arylmethyl-, arylethyl-, arylpropyl-, arylcarbonyl-, arylsulfonyl-, heterocyclemethyl-, heterocycle-ethyl-, heterocyclepropyl-, heterocyclecarbonyl-, and heterocyclesulfonyl- can be unsubstituted or substituted with one or more substituents selected from R⁸; or R² is taken together with R⁵ to form a 5, 6, or 7 membered heterocycle which can be unsubstituted or substituted with one or more substituents selected from R⁸.
46. The compounds according to any one of statements 1 to 31, 34 to 45, wherein R² is independently selected from unsubstituted or substituted with one or more substituents selected from R⁸ benzyl; pyridylmethyl-; furylmethyl-; thienylmethyl-; napthylmethyl-; phenethyl-; indanyl; cyclohexylmethyl-; pyranylmethyl; isoxazolylmethyl-; pyrazolylmethyl-; cyclopentylmethyl-; pyranylethyl-; benzothiophenylmethyl-; indolylmethyl-; tetrahydronaphthylmethyl-; pyrrolidinylmethyl-; quinolylmethyl-; indolinylmethyl-; benzimidazolylmethyl-; dihydrobenzo[b][1,4]dioxinylmethyl-; phenylpropyl; or R² is taken together with R⁵ to form a 5, 6, or 7 membered heterocycle which can be unsubstituted or substituted with one or more substituents selected from W.
47. The compounds according to any one of statements 1 to 31, 34 to 46, wherein R² is independently selected from unsubstituted or substituted with one or more substituents selected from R⁸ benzyl; and pyridylmethyl-.
48. The compounds according to any one of statements 1 to 31, 34 to 47, wherein R⁵ is selected from hydrogen and alkyl. In a more particular embodiment of the invention, R⁵ is selected from hydrogen and C₁₋₉alkyl, more in particular from hydrogen and C₁₋₆alkyl, still more in particular from hydrogen and C₁₋₃alkyl.
49. The compounds according to any one of statements 1 to 31, 34 to 48, wherein R⁵ is selected from hydrogen, methyl, ethyl and propyl.
50. The compounds according to any one of statements 1 to 31, 34 to 49, wherein R⁵ is selected from hydrogen and C₁₋₃alkyl, yet more in particular R⁵ is selected from C₁₋₃alkyl, still more in particular, R⁵ is methyl.
51. The compounds according to any one of statements 1 to 31, 34 to 50, wherein R⁶ is selected from hydrogen, C₁₋₉ alkyl, and arylC₁₋₆alkyl, more in particular from hydrogen and C₁₋₆ alkyl; still more in particular from hydrogen and C₁₋₃ alkyl.
52. The compounds according to any one of statements 1 to 31, 34 to 51, wherein R⁶ is selected from hydrogen, and C₁₋₉ alkyl, still more in particular from hydrogen and C₁₋₃ alkyl.
53. The compounds according to any one of statements 1 to 31, 34 to 52, wherein R⁶ is hydrogen.
54. The compounds according to any one of statements 12, 23 to 31, 34 to 53, wherein X is selected from C₁₋₆ alkylene, a single bond, —C(═O)—, and —SO₂—.
55. The compounds according to any one of statements 7, 12, 23 to 31, 34 to 54, wherein X is selected from C₁₋₆ alkylene, and a single bond.
56. The compounds according to any one of statements 7, 12, 23 to 31, 34 to 55, wherein X is selected from C₁₋₃ alkylene and representing a single bond.
57. The compounds according to any one of statements 7, 12, 23 to 31, 34 to 56, wherein X is methylene.
58. The compounds according to any one of statements 7, 12, 23 to 31, 34 to 54, wherein X is CO.
59. The compounds according to any one of statements 7, 12, 23 to 31, 34 to 54, wherein X is SO₂.
60. The compounds according to any one of statements 1 to 31, 34 to 59, wherein R⁸ is selected from alkyl; heteroalkyl; hydroxyl; halogen; —SH; trifluoromethyl; —OCF₃; cyano; and NH₂.
61. The compounds according to any one of statements 1 to 31, 34 to 60, wherein R⁸ is selected from C₁₋₆alkyl; C₁₋₆heteroalkyl; hydroxyl; halogen; —SH; trifluoromethyl; —OCF₃; cyano; and NH₂.
62. The compounds according to any one of statements 1 to 31, 34 to 61, wherein R⁸ is selected from C₁₋₆alkyl; C₁₋₆heteroalkyl; halogen; trifluoromethyl; —OCF₃; and cyano.
63. The compounds according to any one of statements 1 to 31, 34 to 62, wherein R⁸ is selected from C₁₋₄alkyl; C₁₋₄heteroalkyl; halogen; trifluoromethyl; —OCF₃; and cyano.
64. The compounds according to any one of statements 1 to 31, 34 to 63, wherein R⁸ is selected from methyl; ethyl; propyl; butyl; methoxy; ethoxy; propyloxy; F; Cl; trifluoromethyl; —OCF₃; and cyano.
65. A compound selected from the group comprising 7-((3,5-bis(trifluoromethyl)benzyl)oxy)-2-oxo-2H-chromene-3-carboxylic acid;
7-(naphthalen-1-ylmethoxy)-2-oxo-2H-chromene-3-carboxylic acid;
7-((3-fluorobenzyl)oxy)-2-oxo-2H-chromene-3-carboxylic acid;
7-((3-methoxybenzyl)oxy)-2-oxo-2H-chromene-3-carboxylic acid;
7-((2,5-dimethoxybenzyl)oxy)-2-oxo-2H-chromene-3-carboxylic acid;
7-((1H-benzo[d][1,2,3]triazol-1-yl)methoxy)-2-oxo-2H-chromene-3-carboxylic acid;
2-oxo-7-((4-(trifluoromethyl)benzyl)oxy)-2H-chromene-3-carboxylic acid;
7-((2,3-dihydro-1H-inden-2-yl)oxy)-2-oxo-2H-chromene-3-carboxylic acid;
7-(benzyl(methyl)amino)-2-oxo-2H-chromene-3-carboxylic acid;
2-oxo-7-(piperidin-1-yl)-2H-chromene-3-carboxylic acid;
2-oxo-7-(pyrrolidin-1-yl)-2H-chromene-3-carboxylic acid;
7-(benzylamino)-2-oxo-2H-chromene-3-carboxylic acid;
7-(benzyl(ethyl)amino)-2-oxo-2H-chromene-3-carboxylic acid;
7-(benzyl(isobutyl)amino)-2-oxo-2H-chromene-3-carboxylic acid;
7-(benzyl(propyl)amino)-2-oxo-2H-chromene-3-carboxylic acid;
7-(isobutyl(methyl)amino)-2-oxo-2H-chromene-3-carboxylic acid;
7-(methyl(thiophen-2-ylmethyl)amino)-2-oxo-2H-chromene-3-carboxylic acid;
7-((3-fluorobenzyl)(methyl)amino)-2-oxo-2H-chromene-3-carboxylic acid;
7-((cyclohexylmethyl)(methyl)amino)-2-oxo-2H-chromene-3-carboxylic acid;
7-((3-methoxybenzyl)(methyl)amino)-2-oxo-2H-chromene-3-carboxylic acid;
7-(methyl((tetrahydro-2H-pyran-4-yl)methyl)amino)-2-oxo-2H-chromene-3-carboxylic acid;
7-(dibenzylamino)-2-oxo-2H-chromene-3-carboxylic acid;
and 7-benzamido-2-oxo-2H-chromene-3-carboxylic acid.
66. The compounds according to any one of statements 7 to 31, 34 to 65, for use as a medicine.
67. The compounds according to any one of statements 1 to 31, 34 to 66, for use as in the prevention and/or treatment of cancer in a subject.
68. The compounds according to statement 67, wherein the cancer is a solid cancer.
69. The compounds according to statement 67, wherein the cancer is a cancer expressing MCT1 and/or MCT4.
70. The compounds according to statement 69, wherein the cancer is a solid cancer expressing MCT1 and/or MCT4.
71. The compounds according to any one of statements 67 to 70, wherein the cancer is selected from cervix cancer and colon cancer.
72. A pharmaceutical composition comprising the compounds according to any one of statements 1 to 31, 34 to 71 in combination with a pharmaceutically acceptable carrier.
73. A method for the prevention or treatment of a cancer in an animal, mammal or human comprising administering to said animal, mammal or human in need for such prevention or treatment an effective dose of the compounds according to any one of statements 1 to 31, 34 to 71.
74. A method for the prevention or treatment of a cancer in a subject in need thereof comprising administering to said subject in need for such prevention or treatment an effective dose of the compounds according to any one of statements 1 to 31, 34 to 71.
75. A method to inhibit lactate uptake in vitro or in isolated cells or in an animal, mammal or human by using the compounds according to any one of statements 1 to 31, 34 to 71.
76. The compounds according to any one of statements 1 to 31, 34 to 71, for use in the treatment or prophylaxis of cancer, in combination with other therapeutic agents for the treatment or prophylaxis of cancer.
77. The compounds according to any one of statements 1 to 31, 34 to 71, for use in the treatment or prophylaxis of cancer, in a composition comprising one or more compounds according to any of one of statements 1 to 68, and one or more further therapeutic or preventive agents that are used for the prevention or treatment of cancer as biologically active agents in the form of a combined preparation for simultaneous, separate or sequential use.

In another particular embodiment, the compounds of the invention have a structure according to formula (B), and isomers (in particular stereo-isomers or tautomers), solvates, salts (in particular pharmaceutically acceptable salts) or prodrugs thereof,

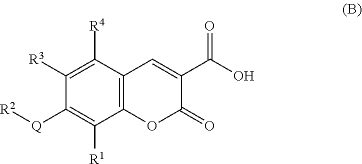

wherein, each of R¹, R³, R⁴, Q and R² is as in formula (A).

In another particular embodiment, the compounds of the invention have a structure according to formula (C), and isomers (in particular stereo-isomers or tautomers), solvates, salts (in particular pharmaceutically acceptable salts) or prodrugs thereof,

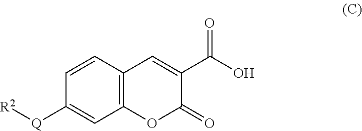

wherein, each of Q and R² is as in formula (A).

In another particular embodiment, the compounds of the invention have a structure according to formula (D1) or (D2), and isomers (in particular stereo-isomers or tautomers), solvates, salts (in particular pharmaceutically acceptable salts) or prodrugs thereof,

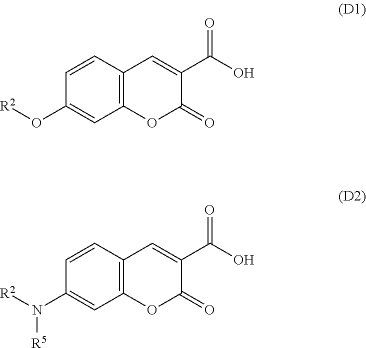

wherein, each of R² and R⁵ isas in formula (A).

In another particular embodiment, the compounds of the invention have a structure according to formula (E1), (E2), (E3) and (E4) and isomers (in particular stereo-isomers or tautomers), solvates, salts (in particular pharmaceutically acceptable salts) or prodrugs thereof,

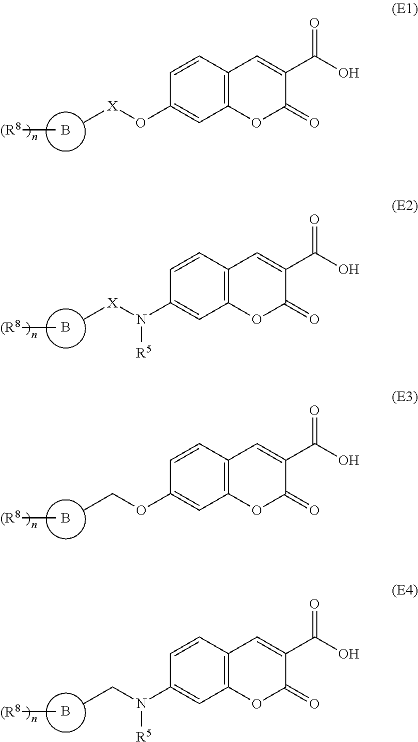

wherein,
each R⁵ and R⁸ is as in formula (A);
n is selected from 0; 1; 2; 3 and 4;
X is selected from alkylene or represents a single bond (thereby establishing a direct bond between 0 and cycle B for formula (E1) or N and cycle B for formula (E2)); and
cycle B is selected from cycloalkyl; cycloalkenyl; cycloalkynyl; aryl; and heterocycle.

In another particular embodiment, the compounds of the invention have a structure according to formula (F1) or (F2), and isomers (in particular stereo-isomers or tautomers), solvates, salts (in particular pharmaceutically acceptable salts) or prodrugs thereof,

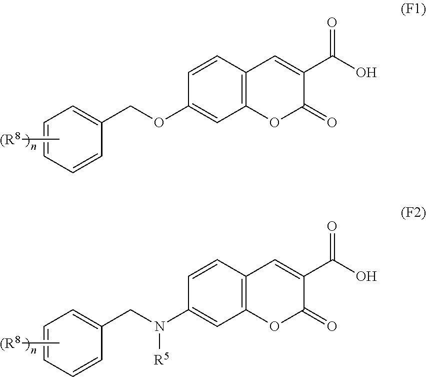

wherein, each R⁵ and R⁸ is as in formula (A) and n is selected from 0; 1; 2; 3 and 4.

In a particular embodiment, each R¹, R³ and R⁴ is independently selected from hydrogen; halogen; trifluoromethyl; trifluoromethoxy; cyano; alkyl; and heteroalkyl; wherein said alkyl, and heteroalkyl can be unsubstituted or substituted with one or more substituents selected from R⁷. In another particular embodiment, each R¹, R³ and R⁴ is independently selected from hydrogen; halogen; trifluoromethyl; trifluoromethoxy; cyano; C₁₋₆ alkyl; and C₁₋₆ heteroalkyl; wherein said C₁₋₆ alkyl; and C₁₋₆ heteroalkyl can be unsubstituted or substituted with one or more substituents selected from R⁷. In yet another particular embodiment, each R¹, R³ and R⁴ is independently selected from hydrogen; halogen; trifluoromethyl; trifluoromethoxy; cyano; C₁₋₄ alkyl; and C₁₋₄ heteroalkyl. In still another particular embodiment, each R¹, R³ and R⁴ is independently selected from hydrogen; F; Cl; trifluoromethyl; trifluoromethoxy; cyano; methyl; ethyl; propyl; butyl; methoxy; ethoxy; propyloxy; and dimethylaminomethylene. In another more particular embodiment, each R¹, R³ and R⁴ is independently selected from hydrogen; F; Cl; trifluoromethyl; trifluoromethoxy; cyano; methyl; ethyl; methoxy; and ethoxy.

In another particular embodiment, R² is independently selected from C₁₋₉ alkyl; C₃₋₉ cycloalkyl; C₂₋₉ alkenyl; C₃₋₉ cycloalkenyl; C₂₋₉ alkynyl; C₃₋₉ cycloalkynyl; C₁₋₉ heteroalkyl; C₂₋₉ heteroalkenyl; C₂₋₉ heteroalkynyl; aryl; heterocycle; aryl C₁₋₉ alkyl; aryl C₂₋₉ alkenyl; aryl C₂₋₉ alkynyl; aryl C₁₋₉ heteroalkyl; aryl C₂₋₉ heteroalkenyl; aryl C₂₋₉ heteroalkynyl; heterocycle-C₁₋₉ alkyl; heterocycle-C₂₋₉ alkenyl; heterocycle-C₂₋₉ alkynyl; heterocycle-C₁₋₉ heteroalkyl; heterocycle-C₂₋₉ heteroalkenyl; or heterocycle-C₂₋₉ heteroalkynyl;
and wherein said C₁₋₉ alkyl, C₃₋₉ cycloalkyl, C₂₋₉ alkenyl, C₃₋₉ cycloalkenyl, C₂₋₉ alkynyl, C₃₋₉ cycloalkynyl, C₁₋₉ heteroalkyl, C₂₋₉ heteroalkenyl, C₂₋₉ heteroalkynyl, aryl, heterocycle, aryl C₁₋₉ alkyl, aryl C₂₋₉ alkenyl, aryl C₂₋₉ alkynyl, aryl C₁₋₉ heteroalkyl, aryl C₂₋₉ heteroalkenyl, aryl C₂₋₉ heteroalkynyl, heterocycle-C₁₋₉ alkyl, heterocycle-C₂₋₉ alkenyl, heterocycle-C₂₋₉ alkynyl, heterocycle-C₁₋₉ heteroalkyl, heterocycle-C₂₋₉ heteroalkenyl, or heterocycle-C₂₋₉ heteroalkynyl can be unsubstituted or substituted with one or more substituents selected from R⁸; or R² is taken together with R⁵ to form a 5, 6, or 7 membered heterocycle which can be unsubstituted or substituted with one or more substituents selected from R⁸.

In yet another particular embodiment, R² is independently selected from C₁₋₉ alkyl; C₃₋₉ cycloalkyl; C₁₋₉ heteroalkyl; aryl; heterocycle; aryl C₁₋₉ alkyl; aryl C₁₋₉ heteroalkyl; heterocycle-C₁₋₉ alkyl; heterocycle-C₁₋₉ heteroalkyl;
and wherein said C₁₋₉ alkyl, C₃₋₉ cycloalkyl, C₁₋₉ heteroalkyl, aryl, heterocycle, aryl C₁₋₉ alkyl, aryl C₁₋₉ heteroalkyl, heterocycle-C₁₋₉ alkyl and heterocycle-C₁₋₉ heteroalkyl, can be unsubstituted or substituted with one or more substituents selected from R⁸. R² is taken together with R⁵ to form a 5, 6, or 7 membered heterocycle which can be unsubstituted or substituted with one or more substituents selected from R⁸.

In still another more particular embodiment, R² is independently selected from aryl C₁₋₉ alkyl; aryl C₁₋₉ heteroalkyl, heterocycle-C₁₋₉ alkyl and heterocycle-C₁₋₉ heteroalkyl; wherein said aryl C₁₋₉ alkyl, aryl C₁₋₉ heteroalkyl, heterocycle-C₁₋₉ alkyl and heterocycle-C₁₋₉ heteroalkyl, can be unsubstituted or substituted with one or more substituents selected from R⁸; or R² is taken together with R⁵ to form a 5, 6, or 7 membered heterocycle which can be unsubstituted or substituted with one or more substituents selected from R⁸.

In still another more particular embodiment, R² is independently selected from arylmethyl-; arylethyl-; arylpropyl-; arylcarbonyl-; arylsulfonyl-; heterocyclemethyl-; heterocycle-ethyl-; heterocyclepropyl-; heterocyclecarbonyl-; and heterocyclesulfonyl-; wherein said arylmethyl-, arylethyl-, arylpropyl-, arylcarbonyl-, arylsulfonyl-, heterocyclemethyl-, heterocycle-ethyl-, heterocyclepropyl-, heterocyclecarbonyl-, and heterocyclesulfonyl- can be unsubstituted or substituted with one or more substituents selected from R⁸; or R² is taken together with R⁵ to form a 5, 6, or 7 membered heterocycle which can be unsubstituted or substituted with one or more substituents selected from R⁸.

In yet another more particular embodiment, R² is independently selected from unsubstituted or substituted with one or more substituents selected from R⁸ benzyl; pyridylmethyl-; furylmethyl-; thienylmethyl-; napthylmethyl-; phenethyl-; indanyl; cyclohexylmethyl-; pyranylmethyl; isoxazolylmethyl-; pyrazolylmethyl-; cyclopentylmethyl-; pyranylethyl-; benzothiophenylmethyl-; indolylmethyl-; tetrahydronaphthylmethyl-; pyrrolidinylmethyl-; quinolylmethyl-; indolinylmethyl-; benzimidazolylmethyl-; dihydrobenzo[b][1,4]dioxinylmethyl-; phenylpropyl; or R² is taken together with R⁵ to form a 5, 6, or 7 membered heterocycle which can be unsubstituted or substituted with one or more substituents selected from R⁸.

In still another more particular embodiment, R² is independently selected from unsubstituted or substituted with one or more substituents selected from R⁸ benzyl; and pyridylmethyl-.

In a particular embodiment of the invention, R⁵ is selected from hydrogen and alkyl. In a more particular embodiment of the invention, R⁵ is selected from hydrogen and C₁₋₉alkyl, more in particular from hydrogen and C₁₋₆alkyl, still more in particular from hydrogen and C₁₋₃alkyl. In another particular embodiment, R⁵ is selected from hydrogen, methyl, ethyl and propyl. In yet another particular embodiment, R⁵ is selected from hydrogen and C₁₋₃alkyl, yet more in particular R⁵ is selected from C₁₋₃alkyl, still more in particular, R⁵ is methyl.

In a particular embodiment of the invention, R⁶ is selected from hydrogen, C₁₋₉ alkyl, and arylC₁₋₆alkyl, more in particular from hydrogen and C₁₋₆ alkyl; still more in particular from hydrogen and C₁₋₃ alkyl. In a very particular embodiment of the invention, R⁶ is hydrogen.

In a particular embodiment of the invention, X is selected from C₁₋₆ alkylene and representing a single bond. In a more particular embodiment, X is selected from C₁₋₃ alkylene and representing a single bond. In a more particular embodiment, X is methylene.

In a particular embodiment of the invention, R⁸ is selected from alkyl; heteroalkyl; hydroxyl; halogen; —SH; trifluoromethyl; —OCF₃; cyano; and NH₂. In a more particular embodiment, R⁸ is selected from C₁₋₆alkyl; C₁₋₆heteroalkyl; hydroxyl; halogen; —SH; trifluoromethyl; —OCF₃; cyano; and NH₂. Yet in a more particular embodiment, R⁸ is selected from C₁₋₆alkyl; C₁₋₆heteroalkyl; halogen; trifluoromethyl; —OCF₃; and cyano. Still in a more particular embodiment, R⁸ is selected from C₁₋₄alkyl; C₁₋₄heteroalkyl; halogen; trifluoromethyl; —OCF₃; and cyano. In a still more particular embodiment, R⁸ is selected from methyl; ethyl; propyl; butyl; methoxy; ethoxy; propyloxy; F; Cl; trifluoromethyl; —OCF₃; and cyano.

A particular embodiment of the present invention relates to the compounds selected from the compounds named in Table 1 of the present application.

A very particular embodiment of the invention relates to the compounds selected from:
7-((3,5-bis(trifluoromethyl)benzyl)oxy)-2-oxo-2H-chromene-3-carboxylic acid;
7-(naphthalen-1-ylmethoxy)-2-oxo-2H-chromene-3-carboxylic acid;
7-((3-fluorobenzyl)oxy)-2-oxo-2H-chromene-3-carboxylic acid;
7-((3-methoxybenzyl)oxy)-2-oxo-2H-chromene-3-carboxylic acid;
7-((2,5-dimethoxybenzyl)oxy)-2-oxo-2H-chromene-3-carboxylic acid;
7-((1H-benzo[d][1,2,3]triazol-1-yl)methoxy)-2-oxo-2H-chromene-3-carboxylic acid;
2-oxo-7-((4-(trifluoromethyl)benzyl)oxy)-2H-chromene-3-carboxylic acid;
7-((2,3-dihydro-1H-inden-2-yl)oxy)-2-oxo-2H-chromene-3-carboxylic acid;
7-(benzyl(methyl)amino)-2-oxo-2H-chromene-3-carboxylic acid;
2-oxo-7-(piperidin-1-yl)-2H-chromene-3-carboxylic acid;
2-oxo-7-(pyrrolidin-1-yl)-2H-chromene-3-carboxylic acid;
7-(benzylamino)-2-oxo-2H-chromene-3-carboxylic acid;
7-(benzyl(ethyl)amino)-2-oxo-2H-chromene-3-carboxylic acid;
7-(benzyl(isobutyl)amino)-2-oxo-2H-chromene-3-carboxylic acid;
7-(benzyl(propyl)amino)-2-oxo-2H-chromene-3-carboxylic acid;
7-(isobutyl(methyl)amino)-2-oxo-2H-chromene-3-carboxylic acid;
7-(methyl(thiophen-2-ylmethyl)amino)-2-oxo-2H-chromene-3-carboxylic acid;
7-((3-fluorobenzyl)(methyl)amino)-2-oxo-2H-chromene-3-carboxylic acid;
7-((cyclohexylmethyl)(methyl)amino)-2-oxo-2H-chromene-3-carboxylic acid;
7-((3-methoxybenzyl)(methyl)amino)-2-oxo-2H-chromene-3-carboxylic acid;
7-(methyl((tetrahydro-2H-pyran-4-yl)methyl)amino)-2-oxo-2H-chromene-3-carboxylic acid; 7-(dibenzylamino)-2-oxo-2H-chromene-3-carboxylic acid;
and 7-benzamido-2-oxo-2H-chromene-3-carboxylic acid.

Another aspect of the invention relates to the compounds described in the first aspect with the formulas (A), (B), (C), (D1), (D2), (E1), (E2), (E3), (E4), (F1), and (F2), and all embodiments thereof, for use as a medicine or a medicament.

Yet another aspect of the invention relates to the compounds described herein for use in the prevention or treatment of a cancer in a subject (selected from an animal, mammal or human). In a particular embodiment, the cancer is selected from a solid cancer. In another particular embodiment, the cancer is selected from a cancer which expresses MCT1 and/or MCT4, more in particular expresses MCT1 and/or MCT4 and MCT1 and/or MCT4 are functionally present in the cell membrane of the cancer cells.

Another embodiment of the invention relates to the compounds described herein for use as lactate influx inhibitors or lactate uptake inhibitors.

The present invention also relates to the use of the compounds herein described for the manufacture of a medicament, in a particular embodiment said medicament is for the prevention or treatment of a cancer in an animal, mammal or human.

Another aspect of the present invention relates to a pharmaceutical composition comprising the compounds described herein above and all embodiments thereof in combination with a pharmaceutically acceptable carrier. The pharmaceutical composition comprises compounds selected from the formulas (A), (B), (C), (D1), (D2), (E1), (E2), (E3), (E4), (F1), and (F2), and all embodiments thereof.

Yet another aspect of the present invention relates to a method for the prevention or treatment of cancer in a subject comprising administering to said subject in need for such prevention or treatment an effective dose of the compounds of the first aspect and the embodiments thereof.

The invention also provides a method to inhibit lactate uptake in vitro or in isolated cells or in an animal, mammal or human by using the compounds described herein and in the embodiments.

Still another aspect relates to a method for the preparation of the compounds of the invention, comprising the steps of
preparing a substituted or unsubstituted 2,4-dihydrobenzaldehyde derivative,
reacting the previously obtained 2,4-dihydrobenzaldehyde derivative with a suitable malonate ester to obtain 7-hydroxy-2-oxo-2H-chromene-3-carboxylate ester derivative,
substitute the previously obtained 7-hydroxy-2-oxo-2H-chromene-3-carboxylate ester derivative with suitable derivatives bearing leaving groups or alcohol derivatives under Mistunobu conditions to obtain 7-O-substituted-2-oxo-2H-chromene-3-carboxylate ester derivative,
converting the previously obtained 7-O-substituted-2-oxo-2H-chromene-3-carboxylate ester derivative in carboxylic acid to obtain the desired compound.

In another embodiment, the invention relates to a method for the preparation of the compounds of the invention, comprising the steps of
preparing a substituted or unsubstituted 3-amino-(mono or disubstituted)-phenol derivative,
reacting the previously obtained 3-amino-(mono or disubstituted)-phenol derivative with a suitable 2-(ethoxymethylene)malonate ester derivative to obtain 7-amino-(mono or disubstituted)-2-oxo-2H-chromene-3-carboxylate ester derivative,
converting the previously obtained 7-amino-(mono or disubstituted)-2-oxo-2H-chromene-3-carboxylate ester derivative in carboxylic acid to obtain the desired compound.

In another embodiment, the invention relates to a method for the preparation of the compounds of the invention, comprising the steps of
preparing a 3-amino-(disubstituted)-phenol derivative,
reacting this 3-amino-(mono or disubstituted)-phenol derivative with dimethylformamide to obtain 4-amino-(mono or disubstituted)-2-hydroxybenzaldehyde derivative,
reacting the previously obtained 4-amino-(mono or disubstituted)-2-hydroxybenzaldehyde derivative with a suitable malonate ester derivative to obtain 7-amino-(mono or disubstituted)-2-oxo-2H-chromene-3-carboxylate ester derivative,
converting the previously obtained 7-amino-(mono or disubstituted)-2-oxo-2H-chromene-3-carboxylate ester derivative in desired 7-amino-(mono or disubstituted)-2-oxo-2H-chromene-3-carboxylic acid derivative.

In another embodiment, the invention relates to a method for the preparation of the compounds of the invention, comprising the steps of
reacting a 4-amino-(mono or disubstituted)-2-hydroxybenzaldehyde derivative with the Meldrum's acid to obtain the desired 7-amino-(mono or disubstituted)-2-oxo-2H-chromene-3-carboxylic acid derivative.

It is to be noticed that the term “comprising”, used in the claims, should not be interpreted as being restricted to the means listed thereafter; it does not exclude other elements or steps.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments. Also embodiments described for an aspect of the invention may be used for another aspect of the invention and can be combined. Where an indefinite or definite article is used when referring to a singular noun e.g. “a” or “an”, “the”, this includes a plural of that noun unless something else is specifically stated.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects.

In each of the following definitions, the number of carbon atoms represents the maximum number of carbon atoms generally optimally present in the substituent or linker; it is understood that where otherwise indicated in the present application, the number of carbon atoms represents the optimal maximum number of carbon atoms for that particular substituent or linker.

The term “leaving group” or “LG” as used herein means a chemical group which is susceptible to be displaced by a nucleophile or cleaved off or hydrolyzed in basic or acidic conditions. In a particular embodiment, a leaving group is selected from a halogen atom (e.g., Cl, Br, I) or a sulfonate (e.g., mesylate, tosylate, triflate).

The term “protecting group” refers to a moiety of a compound that masks or alters the properties of a functional group or the properties of the compound as a whole. The chemical substructure of a protecting group varies widely. One function of a protecting group is to serve as intermediates in the synthesis of the parental drug substance. Chemical protecting groups and strategies for protection/deprotection are well known in the art. See: “Protective Groups in Organic Chemistry”, Theodora W. Greene (John Wiley & Sons, Inc., New York, 1991. Protecting groups are often utilized to mask the reactivity of certain functional groups, to assist in the efficiency of desired chemical reactions, e.g. making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g. alcohols, be physiologically acceptable, although in general it is more desirable if the products are pharmacologically innocuous.

The term “hydrocarbyl”, “C₁₋₁₈ hydrocarbyl”, “hydrocarbyl group” or “C₁₋₁₈ hydrocarbyl group” as used herein refers to C₁-C₁₈ normal, secondary, tertiary, unsaturated or saturated, non-aromatic, acyclic or cyclic, hydrocarbons and combinations thereof. This term therefore comprises alkyl, alkenyl, alkynyl, cycloalkyl, cycloalkenyl and cycloalkynyl.

The terminology “heterohydrocarbyl”, “hetero C₁₋₁₈ hydrocarbyl”, “heterohydrocarbyl group”, “hetero C₁₋₁₈ hydrocarbyl group” or “hydrocarbyl group which optionally includes one or more heteroatoms, said heteroatoms being selected from the atoms consisting of O, S, and N” as used herein, refers to a hydrocarbyl group where one or more carbon atoms are replaced by an oxygen, nitrogen or sulphur atom(s) and thus includes heteroalkyl, heteroalkenyl, heteroalkynyl and non-aromatic heterocycle. This term therefore comprises as an example alkoxy, alkenyloxy, C_{w}alkyl-O—C_{18-w}alkyl, C_{w}alkenyl-O-alkyl, C_{w}alkyl-NH—C_{18-w}alkenyl, among others, wherein w is selected from any number between 1 and 18.

The term “alkyl” or “C₁₋₁₈ alkyl” as used herein means C₁-C₁₈ normal, secondary, or tertiary, linear, branched or straight hydrocarbon with no site of unsaturation. Examples are methyl, ethyl, 1-propyl (n-propyl), 2-propyl (iPr), 1-butyl, 2-methyl-1-propyl(i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-heptyl, n-octyl, n-nonyl, n-decyl, n-undecyl, n-dodecyl, n-tridecyl, n-tetradecyl, n-pentadecyl, n-hexadecyl, n-heptadecyl, and n-octadecyl. In a particular embodiment, the term alkyl refers to C₁₋₁₂ hydrocarbons, more in particular C₁₋₉ hydrocarbons, yet more in particular to C₁₋₆ hydrocarbons, still more in particular to C₁₋₃ hydrocarbons as further defined herein above. A preferred alkyl is C₁₋₉ alkyl, or more in particular C₁₋₆ alkyl. Another preferred alkyl is C₁₋₄ alkyl.

The term “cycloalkyl” or “C₃₋₁₈ cycloalkyl” as used herein and unless otherwise stated means a saturated hydrocarbon monovalent radical having from 3 to 18 carbon atoms consisting of or comprising a C₃₋₁₀ monocyclic or C₇₋₁₈ polycyclic saturated hydrocarbon, such as for instance cyclopropyl, cyclopropylmethylene, cyclobutyl, cyclopentyl, cyclopentylmethylene, cyclopropylethylene, methylcyclopropylene, cyclohexyl, cycloheptyl, cyclooctyl, isopropoylcyclooctyl, cyclooctylmethylene, norbornyl, fenchyl, trimethyltricycloheptyl, decalinyl, adamantyl and the like. For the avoidance of doubt and as an example, cyclopentylmethylene refers to

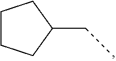

whereby the methyl group on the cyclopentyl is coupled to another group. Furthermore, for the avoidance of doubt and as an example, methylcyclopropylene refers to

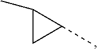

whereby the cyclopropyl of the methylcyclopropyl is coupled to another group. In a particular embodiment, the term cycloalkyl refers to C₃₋₁₂ cycloalkyl, yet more in particular to C₃₋₉ cycloalkyl, yet more in particular to C₃₋₆ cycloalkyl as further defined herein above. A preferred cycloalkyl is C₃₋₉ cycloalkyl. Another preferred cycloalkyl is C₃₋₇cycloalkyl.

The term “alkenyl” or “C₂₋₁₈alkenyl” as used herein is C₂-C₁₈ normal, secondary or tertiary, linear, branched or straight hydrocarbon with at least one site (usually 1 to 3, preferably 1) of unsaturation, namely a carbon-carbon, sp2 double bond. Examples include, but are not limited to: ethylene or vinyl (—CH═CH₂), allyl (—CH₂CH═CH₂), and 5-hexenyl (—CH₂CH₂CH₂CH₂CH═CH₂). The double bond may be in the cis or trans configuration. In a particular embodiment, the term alkenyl refers to C₂₋₁₂ hydrocarbons, yet more in particular to C₂₋₉ hydrocarbons, still more in particular to C₂₋₆ hydrocarbons as further defined herein above. A preferred alkenyl is C₂₋₉ alkenyl, or more in particular C₂₋₆alkenyl. Another preferred alkenyl is C₂₋₄alkenyl.

The term “cycloalkenyl” as used herein refers to a non-aromatic hydrocarbon radical having from 3 to 18 carbon atoms with at least one site (usually 1 to 3, preferably 1) of unsaturation, namely a carbon-carbon, sp2 double bond and consisting of or comprising a C₃₋₁₀ monocyclic or C₇₋₁₈ polycyclic hydrocarbon. Examples include, but are not limited to: cyclopentenyl (—C₅H₇), cyclopentenylpropylene, methylcyclohexenylene and cyclohexenyl (—C₆H₉). The double bond may be in the cis or trans configuration. In a particular embodiment, the term cycloalkenyl refers to C₃₋₁₂ hydrocarbon, yet more in particular to C₃₋₉ hydrocarbon, yet more in particular to C₃₋₆ hydrocarbon as further defined herein above. A preferred cycloalkenyl is C₃₋₉cycloalkenyl. Another preferred cycloalkenyl is C₃₋₇cycloalkenyl. Another preferred cycloalkenyl is C₅₋₉cycloalkenyl. Another preferred cycloalkenyl is C₅₋₇cycloalkenyl.

The term “alkynyl” or “C₂₋₁₈alkynyl” as used herein refers to C₂-C₁₈ normal, secondary, tertiary, linear, branched or straight hydrocarbon with at least one site (usually 1 to 3, preferably 1) of unsaturation, namely a carbon-carbon, sp triple bond. Examples include, but are not limited to: ethynyl (—C≡CH), and 1-propynyl (propargyl, —CH₂C≡CH). In a particular embodiment, the term alkynyl refers to C₂₋₁₂ hydrocarbons, yet more in particular to C₂₋₆ hydrocarbons as further defined herein above. In a particular embodiment, the term alkynyl refers to C₂₋₁₂ hydrocarbons, yet more in particular to C₂₋₉ hydrocarbons, still more in particular to C₂₋₆ hydrocarbons, yet more in particular to C₂₋₄ hydrocarbons as further defined herein above. A preferred alkynyl is C₂₋₉alkynyl, or more in particular C₂₋₆alkynyl. Another preferred alkynyl is C₂₋₄alkynyl.

The term “cycloalkynyl” as used herein refers to a non-aromatic hydrocarbon radical having from 3 to 18 carbon atoms with at least one site (usually 1 to 3, preferably 1) of unsaturation, namely a carbon-carbon, sp triple bond and consisting of or comprising a C₃₋₁₀ monocyclic or C₇₋₁₈ polycyclic hydrocarbon. Examples include, but are not limited to: cyclohept-1-yne, 3-ethyl-cyclohept-1-ynylene, 4-cyclohept-1-yn-methylene and ethylene-cyclohept-1-yne. In a particular embodiment, the term cycloalkyl refers to C₃₋₁₂ cycloalkynyl, yet more in particular to C₃₋₉ cycloalkynyl, yet more in particular to C₃₋₆ cycloalkynyl as further defined herein above. A preferred cycloalkynyl is C₃₋₉cycloalkynyl. Another preferred cycloalkynyl is C₃₋₇cycloalkynyl. Another preferred cycloalkynyl is C₅₋₉cycloalkynyl. Another preferred cycloalkynyl is C₅₋₇cycloalkynyl.

The term “alkylene” as used herein each refer to a saturated, branched or straight chain hydrocarbon radical of 1-18 carbon atoms (more in particular C₁₋₁₂, C₁₋₉ or C₁₋₆ carbon atoms), and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

The term “alkenylene” as used herein each refer to a branched or straight chain hydrocarbon radical of 2-18 carbon atoms (more in particular C₂₋₁₂, C₂₋₉ or Cₘ carbon atoms) with at least one site (usually 1 to 3, preferably 1) of unsaturation, namely a carbon-carbon, sp2 double bond, and having two monovalent radical centers derived by the removal of two hydrogen atoms from the same or two different carbon atoms of a parent alkene.

The term “alkynylene” as used herein each refer to a branched or straight chain hydrocarbon radical of 2-18 carbon atoms (more in particular C₂₋₁₂, C₂₋₉ or C₂₋₆ carbon atoms) with at least one site (usually 1 to 3, preferably 1) of unsaturation, namely a carbon-carbon, sp triple bond, and having two monovalent radical centers derived by the removal of two hydrogen atoms from the same or two different carbon atoms of a parent alkyne.

The term “heteroalkyl” as used herein refers to an alkyl (particularly selected from C₁₋₁₈, C₁₋₁₂, C₁₋₉, C₁₋₈ and C₁₋₄ alkyl) wherein one or more carbon atoms (usually 1, 2 or 3) are replaced by an oxygen, nitrogen or sulphur atom, with the proviso that said chain may not contain two adjacent O atoms or two adjacent S atoms. This means that one or more—CH₃ of said alkyl can be replaced by —NH₂ and/or that one or more—CH₂— of said acyclic alkyl can be replaced by —NH—, —O— or —S—. The S atoms in said chains may be optionally oxidized with one or two oxygen atoms, to afford sulfoxides and sulfones, respectively. Furthermore, the heteroalkyl groups in the compounds of the present invention can contain an oxo or thio group at any carbon or heteroatom that will result in a stable compound. Exemplary heteroalkyl groups include, but are not limited to, alcohols, alkyl ethers, primary, secondary, and tertiary alkyl amines, amides, ketones, esters, alkyl sulfides, and alkyl sulfones.

The term “heteroalkenyl” as used herein refers to an alkenyl (particularly selected from C₂₋₁₈, C₂₋₁₂, C₂₋₉, C₂₋₈ and C₂₋₄ alkenyl) wherein one or more carbon atoms (usually 1, 2 or 3) are replaced by an oxygen, nitrogen or sulphur atom, with the proviso that said chain may not contain two adjacent O atoms or two adjacent S atoms. This means that one or more—CH₃ of said alkenyl can be replaced by —NH₂, that one or more—CH₂— of said acyclic alkenyl can be replaced by —NH—, —O— or —S— and/or that one or more—CH═ of said acyclic alkynyl can be replaced by —N═. The S atoms in said chains may be optionally oxidized with one or two oxygen atoms, to afford sulfoxides and sulfones, respectively. Furthermore, the heteroalkyl groups in the compounds of the present invention can contain an oxo or thio group at any carbon or heteroatom that will result in a stable compound. The term heteroalkenyl thus comprises imines, —O-alkenyl, —NH-alkenyl, —N(alkenyl)₂, —N(alkyl)(alkenyl), and —S-alkenyl.

The term “heteroalkynyl” as used herein refers to an alkynyl (particularly selected from C₂₋₁₈, C₂₋₁₂, C₂₋₉, C₂₋₆ and C₂₋₄ alkynyl) wherein one or more carbon atoms (usually 1, 2 or 3) are replaced by an oxygen, nitrogen or sulphur atom, with the proviso that said chain may not contain two adjacent O atoms or two adjacent S atoms. This means that one or more—CH₃ of said alkynyl can be replaced by —NH₂, that one or more—CH₂— of said alkynyl can be replaced by —NH—, —O— or —S—, that one or more—CH═ of said acyclic alkynyl can be replaced by —N═ and/or that one or more ≡CH of said acyclic alkynyl can be replaced by ≡N. The S atoms in said chains may be optionally oxidized with one or two oxygen atoms, to afford sulfoxides and sulfones, respectively. Furthermore, the heteroalkynyl groups in the compounds of the present invention can contain an oxo or thio group at any carbon or heteroatom that will result in a stable compound. The term heteroalkynyl thus comprises —O— alkynyl, —NH-alkynyl, —N(alkynyl)₂, —N(alkyl)(alkynyl), —N(alkenyl)(alkynyl), and —S-alkynyl.

The term “heteroalkylene” as used herein refers to an alkylene (more in particular C₁₋₁₂, C₁₋₉ or C₁₋₆ alkylene) wherein one or more carbon atoms (usually 1, 2 or 3) are replaced by an oxygen, nitrogen or sulphur atom, with the proviso that said chain may not contain two adjacent O atoms or two adjacent S atoms. This means that one or more—CH₃ of said alkylene can be replaced by —NH₂ and/or that one or more—CH₂— of said alkylene can be replaced by —NH—, —O— or -5-. The S atoms in said chains may be optionally oxidized with one or two oxygen atoms, to afford sulfoxides and sulfones, respectively. Furthermore, the heteroalkylene groups in the compounds of the present invention can contain an oxo or thio group at any carbon or heteroatom that will result in a stable compound.

The term “heteroalkenylene” as used herein refers to an alkenylene (more in particular C₂₋₁₂, C₂₋₉ or C₂₋₆ alkenylene) wherein one or more carbon atoms (usually 1, 2 or 3) are replaced by an oxygen, nitrogen or sulphur atom, with the proviso that said chain may not contain two adjacent O atoms or two adjacent S atoms. This means that one or more—CH₃ of said alkenylene can be replaced by —NH₂, that one or more—CH₂— of said alkenylene can be replaced by —NH—, —O— or —S— and/or that one or more—CH═ of said alkynylene can be replaced by —N═. The S atoms in said chains may be optionally oxidized with one or two oxygen atoms, to afford sulfoxides and sulfones, respectively. Furthermore, the heteroalkenylene groups in the compounds of the present invention can contain an oxo or thio group at any carbon or heteroatom that will result in a stable compound.

The term “heteroalkynylene” as used herein refers to an alkynylene (more in particular C₂₋₁₂, C₂₋₉ or C₂₋₆ alkynylene) wherein one or more carbon atoms (usually 1, 2 or 3) are replaced by an oxygen, nitrogen or sulphur atom, with the proviso that said chain may not contain two adjacent O atoms or two adjacent S atoms. This means that one or more—CH₃ of said alkynylene can be replaced by —NH₂, that one or more—CH₂— of said alkynylene can be replaced by —NH—, —O— or —S—, that one or more—CH═ of said alkynylene can be replaced by —N═ and/or that one or more ≡CH of said alkynylene can be replaced by ≡N. The S atoms in said chains may be optionally oxidized with one or two oxygen atoms, to afford sulfoxides and sulfones, respectively. Furthermore, the heteroalkynylene groups in the compounds of the present invention can contain an oxo or thio group at any carbon or heteroatom that will result in a stable compound.

The term “aryl” as used herein means an aromatic hydrocarbon radical of 6-20 carbon atoms derived by the removal of hydrogen from a carbon atom of a parent aromatic ring system. Typical aryl groups include, but are not limited to 1 ring, or 2 or 3 rings fused together, radicals derived from benzene, naphthalene, anthracene, biphenyl, and the like. The term “parent aromatic ring system” means a monocyclic aromatic ring system or a bi- or tricyclic ring system of which at least one ring is aromatic. Therefore, in this embodiment, typical aryl groups include, but are not limited to 1 ring, or 2 or 3 rings fused together, radicals derived from benzene, naphthalene, anthracene, biphenyl, 2,3-dihydro-1H-indenyl, 5,6,7,8-tetrahydronaphthalenyl, 1,2,6,7,8,8a-hexahydroacenaphthylenyl, 1,2-dihydroacenaphthylenyl, and the like. Particular aryl groups are phenyl and naphthyl, especially phenyl.

The term “arylalkyl” or “arylalkyl-” as used herein refers to an alkyl radical in which one of the hydrogen atoms bonded to a carbon atom, typically a terminal or sp3 carbon atom, is replaced with an aryl radical. Typical arylalkyl groups include, but are not limited to, benzyl, 2-phenylethan-1-yl, 2-phenylethen-1-yl, naphthylmethyl, 2-naphthylethyl, and the like. The arylalkyl group comprises 6 to 20 carbon atoms, e.g. the alkyl moiety of the arylalkyl group is 1 to 6 carbon atoms and the aryl moiety is 5 to 14 carbon atoms.

The term “arylalkenyl” or “arylalkenyl-” as used herein refers to an alkenyl radical in which one of the hydrogen atoms bonded to a carbon atom, is replaced with an aryl radical. The arylalkenyl group comprises 6 to 20 carbon atoms, e.g. the alkenyl moiety of the arylalkenyl group is 1 to 6 carbon atoms and the aryl moiety is 5 to 14 carbon atoms.

The term “arylalkynyl” or “arylalkynyl-” as used herein refers to an alkynyl radical in which one of the hydrogen atoms bonded to a carbon atom, is replaced with an aryl radical. The arylalkynyl group comprises 6 to 20 carbon atoms, e.g. the alkynyl moiety of the arylalkynyl group is 1 to 6 carbon atoms and the aryl moiety is 5 to 14 carbon atoms.

The term “arylheteroalkyl” or “arylheteroalkyl-” as used herein refers to a heteroalkyl radical in which one of the hydrogen atoms bonded to a carbon atom, typically a terminal or sp3 carbon atom, is replaced with an aryl radical. The arylheteroalkyl group comprises 6 to 20 carbon atoms, e.g. the heteroalkyl moiety of the arylheteroalkyl group is 1 to 6 carbon atoms and the aryl moiety is 5 to 14 carbon atoms.

The term “arylheteroalkenyl” or “arylheteroalkenyl-” as used herein refers to a heteroalkenyl radical in which one of the hydrogen atoms bonded to a carbon atom, is replaced with an aryl radical. The arylheteroalkenyl group comprises 6 to 20 carbon atoms, e.g. the heteroalkenyl moiety of the arylheteroalkenyl group is 1 to 6 carbon atoms and the aryl moiety is 5 to 14 carbon atoms.

The term “arylheteroalkynyl” or “arylheteroalkynyl-” as used herein refers to a heteroalkynyl radical in which one of the hydrogen atoms bonded to a carbon atom, is replaced with an aryl radical. The arylheteroalkynyl group comprises 6 to 20 carbon atoms, e.g. the heteroalkynyl moiety of the arylheteroalkynyl group is 1 to 6 carbon atoms and the aryl moiety is 5 to 14 carbon atoms.

The term “heterocycle” as used herein means a saturated, unsaturated or aromatic ring system of 3 to 18 atoms including at least one N, O, S, or P. Heterocycle thus include heteroaryl groups. Heterocycle as used herein includes by way of example and not limitation these heterocycles described in Paquette, Leo A. “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566. In a particular embodiment, the term means pyridyl, dihydroypyridyl, tetrahydropyridyl (piperidyl), thiazolyl, tetrahydrothiophenyl, sulfur oxidized tetrahydrothiophenyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, benzothienyl, benzothiazolyl and isatinoyl.

The term “heteroaryl” means an aromatic ring system of 5 to 18 atoms including at least one N, O, S, or P and thus refers to aromatic heterocycles. Examples of heteroaryl include but are not limited to pyridyl, pyridazinyl, pyrimidinyl, pyrazinyl, s-triazinyl, oxazolyl, imidazolyl, thiazolyl, isoxazolyl, pyrazolyl, isothiazolyl, furyl, thienyl, and pyrrolyl.

The term “non-aromatic heterocycle” as used herein means a saturated or unsaturated non-aromatic ring system of 3 to 18 atoms including at least one N, O, S, or P.

The term “heterocycle-alkyl” or “heterocycle-alkyl-” as used herein refers to an alkyl radical in which one of the hydrogen atoms bonded to a carbon atom, typically a terminal or sp3 carbon atom, is replaced with a heterocycle radical. An example of a heterocycle-alkyl group is 2-pyridyl-methylene. The heterocycle-alkyl group comprises 6 to 20 atoms, e.g. the alkyl moiety of the heterocycle-alkyl group is 1 to 6 carbon atoms and the heterocycle moiety is 3 to 14 atoms.

The term “heterocycle-alkenyl” or “heterocycle-alkenyl-” as used herein refers to an alkenyl radical in which one of the hydrogen atoms bonded to a carbon atom, is replaced with an heterocycle radical. The heterocycle-alkenyl group comprises 6 to 20 atoms, e.g. the alkenyl moiety of the heterocycle-alkenyl group is 1 to 6 carbon atoms and the heterocycle moiety is 3 to 14 atoms.

The term “heterocycle-alkynyl” or “heterocycle-alkynyl-” as used herein refers to an alkynyl radical in which one of the hydrogen atoms bonded to a carbon atom, is replaced with a heterocycle radical. The heterocycle-alkynyl group comprises 6 to 20 atoms, e.g. the alkynyl moiety of the heterocycle-alkynyl group is 1 to 6 carbon atoms and the heterocycle moiety is 3 to 14 atoms.

The term “heterocycle-heteroalkyl” or “heterocycle-heteroalkyl-” as used herein refers to a heteroalkyl radical in which one of the hydrogen atoms bonded to a carbon atom, typically a terminal or sp3 carbon atom, is replaced with a heterocycle radical. The heterocycle-heteroalkyl group comprises 6 to 20 atoms, e.g. the heteroalkyl moiety of the heterocycle-heteroalkyl group is 1 to 6 carbon atoms and the heterocycle moiety is 3 to 14 atoms.

The term “heterocycle-heteroalkenyl” or “heterocycle-heteroalkenyl-” as used herein refers to a heteroalkenyl radical in which one of the hydrogen atoms bonded to a carbon atom, is replaced with an heterocycle radical. The heterocycle-heteroalkenyl group comprises 6 to 20 atoms, e.g. the heteroalkenyl moiety of the heterocycle-heteroalkenyl group is 1 to 6 carbon atoms and the heterocycle moiety is 3 to 14 atoms.

The term “heterocycle-heteroalkynyl” or “heterocycle-heteroalkynyl-” as used herein refers to a heteroalkynyl radical in which one of the hydrogen atoms bonded to a carbon atom, is replaced with a heterocycle radical. The heterocycle-heteroalkynyl group comprises 6 to 20 atoms, e.g. the heteroalkynyl moiety of the heterocycle-heteroalkynyl group is 1 to 6 carbon atoms and the heterocycle moiety is 3 to 14 atoms.

The term “heteroaryl-alkyl” or “heteroaryl-alkyl-” as used herein refers to an alkyl radical in which one of the hydrogen atoms bonded to a carbon atom, typically a terminal or sp3 carbon atom, is replaced with a heteraryl radical. An example of a heteroaryl-alkyl group is 2-pyridyl-methylene. The heteroaryl-alkyl group comprises 6 to 20 atoms, e.g. the alkyl moiety of the heteroaryl-alkyl group is 1 to 6 carbon atoms and the heteroaryl moiety is 5 to 14 atoms.

The term “heteroaryl-alkenyl” or “heteroaryl-alkenyl-” as used herein refers to an alkenyl radical in which one of the hydrogen atoms bonded to a carbon atom, is replaced with an heteroaryl radical. The heteroaryl-alkenyl group comprises 6 to 20 atoms, e.g. the alkenyl moiety of the heteroaryl-alkenyl group is 1 to 6 carbon atoms and the heteroaryl moiety is 5 to 14 atoms.

The term “heteroaryl-alkynyl” or “heteroaryl-alkynyl-” as used herein refers to an alkynyl radical in which one of the hydrogen atoms bonded to a carbon atom, is replaced with a heteroaryl radical. The heteroaryl-alkynyl group comprises 6 to 20 atoms, e.g. the alkynyl moiety of the heteroaryl-alkynyl group is 1 to 6 carbon atoms and the heteroaryl moiety is 5 to 14 atoms.

The term “heteroaryl-heteroalkyl” or “heteroaryl-heteroalkyl-” as used herein refers to a heteroalkyl radical in which one of the hydrogen atoms bonded to a carbon atom, typically a terminal or sp3 carbon atom, is replaced with a heterocycle radical. The heteroaryl-heteroalkyl group comprises 6 to 20 atoms, e.g. the heteroalkyl moiety of the heteroaryl-heteroalkyl group is 1 to 6 carbon atoms and the heteroaryl moiety is 5 to 14 atoms.

The term “heteroaryl-heteroalkenyl” or “heteroaryl-heteroalkenyl-” as used herein refers to a heteroalkenyl radical in which one of the hydrogen atoms bonded to a carbon atom, is replaced with an heteroaryl radical. The heteroaryl-heteroalkenyl group comprises 6 to 20 atoms, e.g. the heteroalkenyl moiety of the heteroaryl-heteroalkenyl group is 1 to 6 carbon atoms and the heteroaryl moiety is 5 to 14 atoms.

The term “heteroaryl-heteroalkynyl” or “heteroaryl-heteroalkynyl-” as used herein refers to a heteroalkynyl radical in which one of the hydrogen atoms bonded to a carbon atom, is replaced with a heteroaryl radical. The heteroaryl-heteroalkynyl group comprises 6 to 20 atoms, e.g. the heteroalkynyl moiety of the heteroaryl-heteroalkynyl group is 1 to 6 carbon atoms and the heteroaryl moiety is 5 to 14 atoms.

The term “non-aromatic heterocycle-alkyl” or “non-aromatic heterocycle-alkyl-” as used herein refers to an acyclic alkyl radical in which one of the hydrogen atoms bonded to a carbon atom, typically a terminal or sp3 carbon atom, is replaced with a non-aromatic heterocycle radical. The non-aromatic heterocycle-alkyl group comprises 6 to 20 atoms, e.g. the alkyl moiety of the non-aromatic heterocycle-alkyl group is 1 to 6 carbon atoms and the non-aromatic heterocycle moiety is 3 to 14 atoms.

The term “non-aromatic heterocycle-alkenyl” or “non-aromatic heterocycle-alkenyl-” as used herein refers to an acyclic alkenyl radical in which one of the hydrogen atoms bonded to a carbon atom, is replaced with an non-aromatic heterocycle radical. The non-aromatic heterocycle-alkenyl group comprises 6 to 20 atoms, e.g. the alkenyl moiety of the non-aromatic heterocycle-alkenyl group is 1 to 6 carbon atoms and the non-aromatic heterocycle moiety is 3 to 14 atoms.

The term “non-aromatic heterocycle-alkynyl” or “non-aromatic heterocycle-alkynyl-” as used herein refers to an acyclic alkynyl radical in which one of the hydrogen atoms bonded to a carbon atom, is replaced with a non-aromatic heterocycle radical. The non-aromatic heterocycle-alkynyl group comprises 6 to 20 atoms, e.g. the alkynyl moiety of the non-aromatic heterocycle-alkynyl group is 1 to 6 carbon atoms and the non-aromatic heterocycle moiety is 3 to 14 atoms.

The term “non-aromatic heterocycle-heteroalkyl” or “non-aromatic heterocycle-heteroalkyl-” as used herein refers to a heteroalkyl radical in which one of the hydrogen atoms bonded to a carbon atom, typically a terminal or sp3 carbon atom, is replaced with a heterocycle radical. The non-aromatic heterocycle-heteroalkyl group comprises 6 to 20 atoms, e.g. the heteroalkyl moiety of the non-aromatic heterocycle-heteroalkyl group is 1 to 6 carbon atoms and the non-aromatic heterocycle moiety is 3 to 14 atoms.

The term “non-aromatic heterocycle-heteroalkenyl” or “non-aromatic heterocycle-heteroalkenyl-” as used herein refers to a heteroalkenyl radical in which one of the hydrogen atoms bonded to a carbon atom, is replaced with an non-aromatic heterocycle radical. The non-aromatic heterocycle-heteroalkenyl group comprises 6 to 20 atoms, e.g. the heteroalkenyl moiety of the non-aromatic heterocycle-heteroalkenyl group is 1 to 6 carbon atoms and the non-aromatic heterocycle moiety is 3 to 14 atoms.

The term “non-aromatic heterocycle-heteroalkynyl” or “non-aromatic heterocycle-heteroalkynyl-” as used herein refers to a heteroalkynyl radical in which one of the hydrogen atoms bonded to a carbon atom, is replaced with a non-aromatic heterocycle radical. The non-aromatic heterocycle-heteroalkynyl group comprises 6 to 20 atoms, e.g. the heteroalkynyl moiety of the non-aromatic heterocycle-heteroalkynyl group is 1 to 6 carbon atoms and the non-aromatic heterocycle moiety is 3 to 14 atoms.

By way of example, carbon bonded heterocyclic rings are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl. By way of example, nitrogen bonded heterocyclic rings are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

As used herein and unless otherwise stated, the terms “alkoxy”, “cyclo-alkoxy”, “aryloxy”, “arylalkyloxy”, “heterocycleoxy”, “alkylthio”, “cycloalkylthio”, “arylthio”, “arylalkylthio” and “heterocyclethio” refer to substituents wherein an alkyl group, respectively a cycloalkyl, aryl, arylalkyl or heterocycle (each of them such as defined herein), are attached to an oxygen atom or a sulfur atom through a single bond, such as but not limited to methoxy, ethoxy, propoxy, butoxy, thioethyl, thiomethyl, phenyloxy, benzyloxy, mercaptobenzyl and the like. The same definitions will apply for alkenyl and alkynyl radicals instead of alkyl. A preferred alkoxy is C₁₋₆alkoxy; another preferred alkoxy is C₁₋₄alkoxy.

As used herein and unless otherwise stated, the term halogen means any atom selected from the group consisting of fluorine (F), chlorine (Cl), bromine (Br) and iodine (I).

As used herein with respect to a substituting group, and unless otherwise stated, the terms “substituted” such as in “substituted alkyl”, “substituted alkenyl”, substituted alkynyl“, “substituted aryl”, “substituted heterocycle”, “substituted arylalkyl”, “substituted heterocycle-alkyl” and the like refer to the chemical structures defined herein, and wherein the said hydrocarbyl, heterohydrocarbyl group and/or the said aryl or heterocycle may be optionally substituted with one or more substituents (preferable 1, 2, 3, 4, 5 or 6), meaning that one or more hydrogen atoms are each independently replaced with a substituent. Typical substituents include, but are not limited to and in a particular embodiment said substituents are being independently selected from the group consisting of halogen, amino, hydroxyl, sulfhydryl, alkyl, alkoxy, alkenyl, alkenyloxy, alkynyl, alkynyloxy, cycloalkyl, cycloalkenyl, cycloalkynyl, heteroalkyl, heteroalkenyl, heteroalkynyl, aryl, heterocycle, arylalkyl, arylalkenyl, arylalkynyl, heterocycle-alkyl, heterocycle-alkenyl and heterocycle-alkynyl, —X, —Z, —O⁻, —OZ, ═O, —SZ, —S⁻, ═S, —NZ₂, —N⁺Z₃, ═NZ, ═N—OZ, —CX₃ (e.g. trifluoromethyl), —CN, —OCN, —SCN, —N═C═O, —N═C═S, —NO, —NO₂, ═N₂, —N₃, —NZC(O)Z, —NZC(S)Z, —NZC(O)O⁻, —NZC(O)OZ, —NZC(S)OZ, —NZC(O)NZZ, NZC(NZ)Z, NZC(NZ)NZZ, —C(O)NZZ, —C(NZ)Z, —S(O)₂O⁻, —S(O)₂OZ, —S(O)₂Z, —OS(O)₂OZ, —OS(O)₂Z, —OS(O)₂O⁻, —S(O)₂NZ, —S(O)Z, —OP(O)(OZ)₂, —P(O)(OZ)₂, —P(O)(O)₂, —P(O)(OZ)(O⁻), —P(O)(OH)₂, —C(O)Z, —C(O)X, —C(S)Z, —C(O)OZ, —C(O)O⁻, —C(S)OZ, —C(O)SZ, —C(S)SZ, —C(O)NZZ, —C(S)NZZ, —C(NZ)NZZ, —OC(O)Z, —OC(S)Z, —OC(O)O⁻, —OC(O)OZ, —OC(S)OZ, wherein each X is independently a halogen selected from F, Cl, Br, or I; and each Z is independently —H, alkyl, alkenyl, alkynyl, heteroalkyl, heteroalkenyl, heteroalkynyl, aryl, heterocycle, protecting group or prodrug moiety, while two Z bonded to a nitrogen atom can be taken together with the nitrogen atom to which they are bonded to form a heterocycle. Alkyl(ene), alkenyl(ene), and alkynyl(ene) groups may also be similarly substituted.

Any substituent designation that is found in more than one site in a compound of this invention shall be independently selected.

Substituents optionally are designated with or without bonds. Regardless of bond indications, if a substituent is polyvalent (based on its position in the structure referred to), then any and all possible orientations of the substituent are intended.

As used herein and unless otherwise stated, the term “solvate” includes any combination which may be formed by a derivative of this invention with a suitable inorganic solvent (e.g. hydrates) or organic solvent, such as but not limited to alcohols, ketones, esters, ethers, nitriles and the like.

The term “heteroatom(s)” as used herein means an atom selected from nitrogen, which can be quaternized; oxygen; and sulfur, including sulfoxide and sulfone.

The term “hydroxy” as used herein means —OH.

The term “carbonyl” as used herein means carbon atom bonded to oxygen with a double bond, i.e., C═O.

The term “amino” as used herein means the —NH₂ group.

The compounds of the invention are lactate influx inhibitors and can be used for the prevention or treatment of cancer or tumor formation in a subject. The present invention provides novel coumarin derivatives, which have been shown that they possess lactate influx inhibitory activity. The present invention furthermore demonstrates that these compounds efficiently inhibit the proliferation of cancer cells when they are dependent on lactate as energy source. The invention also demonstrates that the compounds have an anti-cancer effect in vivo (in xenograft mouse models). Therefore, the compounds of the invention constitute a useful class of new potent compounds that can be used in the treatment and/or prevention of cancer or tumor formation in subjects.

The present invention furthermore relates to the compounds for use as medicines and to their use for the manufacture of medicaments for treating and/or preventing cancer. The invention also relates to methods for the preparation of all such compounds and to pharmaceutical compositions comprising them in an effective amount. The present invention also relates to a method of treatment or prevention of cancer in humans by the administration of one or more such compounds, optionally in combination with one or more other medicines, to a patient in need thereof.

More particularly, the compounds of the invention are coumarin derivatives, more specifically compounds of formula (A) to (F2), and embodiments described herein.

The term “treat” or “treating” as used herein is intended to refer to administration of a compound or composition of the invention to a subject for the purpose of effecting a therapeutic or prophylactic benefit through inhibition of a cancer. Treating includes reversing, ameliorating, alleviating, inhibiting the progress of, lessening the severity of, or preventing a disease, disorder, or condition, or one or more symptoms of such disease, disorder or condition mediated through the inhibition of the cancer.

The term “subject” refers to an animal or mammalian patient in need of such treatment, such as a human.

The term “inhibitors of lactate influx” or “lactate influx inhibitors” refers to compounds that decrease, reduce, prevent, inhibit, partially or fully block the uptake, influx or transport into the cell of lactate by cells.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation or partial alleviation of the symptoms of the disease or disorder being treated.

The term “composition” as used herein is intended to encompass a product comprising the specified ingredients in the therapeutically effective amounts, as well as any product which results, directly or indirectly, from combinations of the specified ingredients in the specified amounts.

The term “cancer” or “tumor formation” refers to a mass of abnormal tissue that arises without obvious cause from pre-existing body cells, has no purposeful function, and is characterized by a tendency to autonomous and unrestrained growth. Examples of tumors or cancers envisaged in the context of the present invention include non-hematological cancers and hematological malignancies and such cancers/malignancies include but are not limited to cancer of the cervix, prostate, lung, breast, rectal, colon, lymph node, bladder, kidney, pancreatic, liver, ovarian, uterine, brain, skin, sarcoma, meningioma, glioblastoma, multiforme, skin, stomach, including all kinds of neuroblastoma, gastric carcinoma, renal cell carcinoma, neuroblastoma, gastric carcinoma, renal cell carcinoma, uterine cancer, muscle cancer or other tumors such as leukemia. The term “solid tumor” refers to non-metastased cancers or benign cancers (for a detailed description see below).

With “hematological malignancies” or “cancers of the blood-forming tissues” are meant cancers such as leukemias (Lymphoblastic T cell leukemia, Chronic myelogenous leukemia (CML), Chronic lymphocytic/lymphoid leukemia (CLL), Hairy-cell leukemia, acute lymphoblastic leukemia (ALL), acute myelogenous leukemia (AML), myelodysplastic syndrome, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, Acute megakaryocytic leukemia, promyelocytic leukemia and Erythroleukemia) and lymphomas, more specifically malignant lymphoma, Hodgkin's lymphoma, non-Hodgkin's lymphoma, lymphoblastic T cell lymphoma, Burkitt's lymphoma and follicular lymphoma, MALT1 lymphomas, Hodgkin lymphomas, B-cell non-Hodgkin lymphoma- and marginal zone lymphoma, among others.

The compounds of the invention have been shown to inhibit cancer or tumor formation. Current research suggests that each tumor arises from a single cell that has been transformed by one or more events. Such events include the activation of oncogenes and the absence or inactivation of specific tumor-suppressor genes. These transformed cells can form small clones, initially co-opting normal host vessels, growing to only several millimeters in size before their supply of nutrients becomes limited. At this point, the tumor may lie dormant for prolonged periods (from months to years) until ultimately undergoing destruction by the immune system or switching to an angiogenic phenotype. This “switch” involves a shift in the local equilibrium between negative and positive endogenous regulators of angiogenesis. The tumor cells may achieve this shift in several ways, including the overexpression of angiogenic factors, the recruitment of host cells (such as macrophages) that can produce their own angiogenic factors, the mobilization of angiogenic proteins from the extracellular matrix (ECM), or a combination of these processes. If the production of proangiogenic factors is sufficiently robust, neighboring endothelial cells will be activated, leading to the sprouting of new capillaries.

Tumors are quite different from inflammatory or other swellings because the cells in tumors are abnormal in their appearance and other characteristics. Abnormal cells—the kind that generally make up tumors—differ from normal cells in having undergone one or more of the following alterations: (1) hypertrophy, or an increase in the size of individual cells; this feature is occasionally encountered in tumors but occurs commonly in other conditions; (2) hyperplasia or an increase in the number of cells within a given zone; in some instances it may constitute the only criterion of tumor formation; (3) anaplasia, or a regression of the physical characteristics of a cell toward a more primitive or undifferentiated type; this is an almost constant feature of malignant tumors, though it occurs in other instances both in health and in disease. In some instances the cells of a tumor are normal in appearance and are faithful reproductions of their parent types so that the differences between them and normal body cells are difficult to discern. Such tumors are also often benign. Other tumors are composed of cells that appear different from normal adult types in size, shape, and structure. They usually belong to tumors that are malignant. Such cells may be bizarre in form or be arranged in a distorted manner. In more extreme cases, the cells of malignant tumors are described as primitive, or undifferentiated, because they have lost the appearance and functions of the particular type of (normal) specialized cell that was their predecessor. As a rule, the less differentiated malignant tumor cells are, the more quickly that tumor may grow. Malignancy refers to the ability of a tumor to ultimately cause death. Any tumor, either benign or malignant in type, may produce death by local effects. The common and more specific definition of malignancy implies an inherent tendency of the tumor's cells to metastasize (invade the body widely and become disseminated by subtle means) and eventually to kill the patient unless all the malignant cells can be eradicated. Metastasis is thus the outstanding characteristic of malignancy. Metastasis is the tendency of tumor cells to be carried from their site of origin by way of the circulatory system and other channels, which may eventually establish these cells in almost every tissue and organ of the body. The amount of new blood vessel growth can correlate with poor prognosis in several tumor types. Since the shedding of large numbers of tumor cells from the primary tumor may not begin until after the tumor has a sufficient network of blood vessels, angiogenesis may also correlate with metastatic potential. Destruction of the ECM is probably necessary to initiate the metastatic process. Microvessel density has been correlated with cancer invasion and metastasis in a number of human tumors including breast, prostate, lung, esophageal, colorectal, endometrial and cervical.

In contrast to malignant tumor cells, the cells of a benign tumor invariably remain in contact with each other in one solid mass centered on the site of origin (“solid tumors”). Because of the physical continuity of benign tumor cells, they may be removed completely by surgery if the location is suitable. But the dissemination of malignant cells, each one individually possessing (through cell division) the ability to give rise to new masses of cells (new tumors) in new and distant sites, precludes complete eradication by a single surgical procedure in all but the earliest period of growth. A benign tumor may undergo malignant transformation, but the cause of such change is unknown. It is also possible for a malignant tumor to remain quiescent, mimicking a benign one clinically, for a long time. All benign tumors tend to remain localized at the site of origin. Many benign tumors are encapsulated. The capsule consists of connective tissue derived from the structures immediately surrounding the tumor.

Well-encapsulated tumors are not anchored to their surrounding tissues. These benign tumors enlarge by accretion, pushing aside the adjacent tissues without involving them intimately.

Among the major types of benign tumors are the following: lipomas, which are composed of fat cells; angiomas, which are composed of blood or lymphatic vessels; osteomas, which arise from bone; chondromas, which arise from cartilage; and adenomas, which arise from glands. For malignant tumors, examples comprise carcinomas (occur in epithelial tissues, which cover the body (the skin) and line the inner cavitary structures of organs (such as the breast, the respiratory and gastrointestinal tracts, the endocrine glands, and the genitourinary system). Sarcomas develop in connective tissues, including fibrous tissues, adipose (fat) tissues, muscle, blood vessels, bone, and cartilage. A cancer can also develop in both epithelial and connective tissue and is called a carcinosarcoma. Cancers of the blood-forming tissues (such as leukemias and lymphomas), tumors of nerve tissues (including the brain), and melanoma (a cancer of the pigmented skin cells) are classified separately.

Hypoxia and oncogenes support the glycolytic pathway as a major energy fuel in tumors. High glucose availability is therefore needed to support the production of ATP and biosynthetic intermediates and high amounts of lactate are consecutively released in the extracellular tumor compartment. In contrast, oxygenated tumor cells can take up lactate and consume it after oxidation to pyruvate by lactate dehydrogenase (Sonveaux et al., J Clin Invest. 2008 December; 118(12):3930-42; Boidot et al., Cancer Res. 2012 Feb. 15; 72(4):939-48). The capacity of oxidative tumor cells to use lactate to fuel the tricarboxylic acid (TCA) cycle (to produce ATP and biosynthetic intermediates) allows hypoxic tumor cells to get access to higher levels of glucose (Sonveaux et al., J Clin Invest. 2008 December; 118(12):3930-42). This metabolic symbiosis was more recently reported to also occur between glycolytic tumor-associated fibroblasts feeding oxidative tumor cells with lactate (Fiaschi et al., Cancer Res. 2012 Oct. 1; 72(19):5130-40; Rattigan et al., Exp Cell Res. 2012 Feb. 15; 318(4):326-35; Whitaker-Menezes et al., Cell Cycle. 2011 Jun. 1; 10(11):1772-83.). Also, a direct pro-angiogenic role of lactate was reported to occur following uptake of lactate by tumor-associated endothelial cells and consecutive prolyl hydroxylase inhibition through direct competition with oxoglutarate (Polet and Feron, J Intern Med. 2013 February; 273(2):156-65). Both NFkB and HIF-1 pathways are consecutively activated leading to the stimulation of angiogenesis. (Vegran et al., Cancer Res. 2011 Apr. 1; 71(7):2550-60; Sonveaux et al., PLoS One. 2012; 7(3):e33418; De Saedeleer et al., PLoS One. 2012; 7(10):e46571)

This lactate-driven cooperativity between a variety of cells within tumors requires the transport of lactate out and into cells. Blocking lactate influx can prevent oxidative tumor cells to use lactate and therefore force them to consume glucose more avidly (Sonveaux et al., J Clin Invest. 2008 December; 118(12):3930-42; Boidot et al., Cancer Res. 2012 Feb. 15; 72(4):939-48). Consequently, hypoxic tumor cells that are essentially dependent on glucose and have limited or no access to replacement fuels die from glucose deprivation. In addition blocking lactate influx in tumor-associated endothelial cells may lead to direct antiangiogenic effects (Vegran et al., Cancer Res. 2011 Apr. 1; 71(7):2550-60; Sonveaux et al., PLoS One. 2012; 7(3):e33418; De Saedeleer et al., PLoS One. 2012; 7(10):e46571), further impacting tumor growth. Blocking lactate influx in tumors is therefore a valid anti-cancer strategy, more specifically to target the difficult to treat hypoxic regions of cancers. Because of the natural organization of tumors in hypoxic and oxygenated compartments and since angiogenesis and fibroblast recruitment in the tumor microenvironment are common characteristics of cancers, virtually all cancer patients may benefit from the administration of lactate influx blockers, more in particularly of the compounds of the invention.

The transport of lactate in and out of cells goes through specific transporters, named MCT (monocarboxylate transporters). The family of MCT (also named SLC16 solute carrier) is composed of 14 members. Among them, only four isoforms (MCT1-4) have been documented to act as proton-linked transporters that can carry short chain monocarboxylates such a as lactate and pyruvate across cell membranes. In cancer cells, MCT1 and MCT4 are the most widely reported. While MCT4 is usually expressed in response to hypoxia and involved in lactate efflux, MCT1, because of its higher affinity for lactate (Km 3-6 mM vs 25-30 mM for MCT4) is a transporter particularly adapted for lactate uptake by oxidative tumor cells.

Particularly envisaged by the present invention are cancers expressing MCT1 and/or MCT4. The expression of MCT1 has been reported in a variety of human cancers including among others colon, breast, head and neck and lung cancers and central nervous system (glioma), breast, lung, gynecologic tract (incl. cervix), prostate, stomach, pancreas and colon cancer.

More generally, the invention relates to the compounds of the formulae described herein and embodiments thereof being useful as agents having biological activity or as diagnostic agents. Any of the uses mentioned with respect to the present invention may be restricted to a non-medical use, a non-therapeutic use, a non-diagnostic use, or exclusively an in vitro use, or a use related to cells remote from an animal. The compounds of the invention can optionally be bound covalently to an insoluble matrix and used for affinity chromatography (separations, depending on the nature of the groups of the compounds, for example compounds with pendant aryl are useful in hydrophobic affinity separations).

When using one or more derivatives of the formulae as defined herein:
the active ingredients of the compound(s) may be administered to the animal or mammal (including a human) to be treated by any means well known in the art, e.g. orally, intranasally, subcutaneously, intramuscularly, intradermally, intravenously, intra-arterially, parenterally or by catheterization.
the therapeutically effective amount of the preparation of the compound(s), especially for the treatment of cancer in humans and other mammals, preferably is a lactate influx inhibiting amount of the compounds of the formulae as defined herein and corresponds to an amount which ensures a plasma level of between 1 μg/ml and 100 mg/ml, optionally of 10 mg/ml.
   Suitable dosages of the compounds or compositions of the invention should be used to treat or prevent the cancer in a subject. Depending upon the pathologic condition to be treated and the patient's condition, the said effective amount may be divided into several sub-units per day or may be administered at more than one day intervals.

The compounds of the invention may be applied as a sole therapy or may involve, in addition to the compound of the invention, conventional surgery or radiotherapy or immunotherapy or hyperthermia or photodynamic therapy or other agents used to prevent or treat cancer (chemotherapy or targeted therapy). According to a particular embodiment of the invention, the compounds of the invention may be employed in combination with other therapeutic agents for the treatment or prophylaxis of cancer. The invention therefore relates to the use of a composition comprising:
(a) one or more compounds of the formulae and embodiments of the invention herein, and
(b) one or more further therapeutic or preventive agents that are used for the prevention or treatment of cancer as biologically active agents in the form of a combined preparation for simultaneous, separate or sequential use.

Examples of such further therapeutic agents or chemotherapy for use in combinations include but are not limited thereto:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin); CHOP regimen (cyclophosphamide, hydroxydoxorubicin, vincristine and prednisone).
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbBl antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD 1839), JV-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN 107); inhibitors of serine/threonine kinases (for example Ras/Raf signalling inhibitors such as farnesyl transferase inhibitors, for example sorafenib (BAY 43-9006), tipifarnib (RI 15777) and lonafarnib (SCH66336)), inhibitors of cell signalling through MEK and/or AKT kinases, c-kit inhibitors, abl kinase inhibitors, Pl3 kinase inhibitors, Plt3 kinase inhibitors, CSF-IR kinase inhibitors, IGF receptor (insulin-like growth factor) kinase inhibitors; aurora kinase inhibitors (for example AZDI 152, PH739358, VX-680, MLN8054, R763, MP235, MP529, VX-528 AND AX39459) and cyclin dependent kinase inhibitors such as CDK2 and/or CDK4 inhibitors; (v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SUI 1248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO02/04434, and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCAI or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies; and
(xi) targeted immune modulation approaches, including for example monoclonal antibodies such as rituximab (rituxan) that targets the CD20 antigen on the surface of malignant and normal B cells and kills tumour cells through complement-dependent cytotoxicity, antibody-dependent cellular cytotoxicity, and induction of apoptosis.

The pharmaceutical composition or combined preparation according to this invention may contain the coumarine derivatives of the present invention over a broad content range depending on the contemplated use and the expected effect of the preparation. Generally, the content of the coumarine derivatives of the present invention of the combined preparation is within the range of 0.1 to 99.9% by weight, preferably from 1 to 99% by weight, more preferably from 5 to 95% by weight.

Those of skill in the art will also recognize that the compounds of the invention may exist in many different protonation states, depending on, among other things, the pH of their environment. While the structural formulae provided herein depict the compounds in only one of several possible protonation states, it will be understood that these structures are illustrative only, and that the invention is not limited to any particular protonation state—any and all protonated forms of the compounds are intended to fall within the scope of the invention.

The term “pharmaceutically acceptable salts” as used herein means the therapeutically active non-toxic salt forms which the compounds of formulae herein are able to form. Therefore, the compounds of this invention optionally comprise salts of the compounds herein, especially pharmaceutically acceptable non-toxic salts containing, for example, Na⁺, Li⁺, K⁺, Ca²⁺ and Mg²⁺. Such salts may include those derived by combination of appropriate cations such as alkali and alkaline earth metal ions or ammonium and quaternary amino ions with an acid anion moiety, typically a carboxylic acid. The compounds of the invention may bear multiple positive or negative charges. The net charge of the compounds of the invention may be either positive or negative. Any associated counter ions are typically dictated by the synthesis and/or isolation methods by which the compounds are obtained. Typical counter ions include, but are not limited to ammonium, sodium, potassium, lithium, halides, acetate, trifluoroacetate, etc., and mixtures thereof. It will be understood that the identity of any associated counter ion is not a critical feature of the invention, and that the invention encompasses the compounds in association with any type of counter ion. Moreover, as the compounds can exist in a variety of different forms, the invention is intended to encompass not only forms of the compounds that are in association with counter ions (e.g., dry salts), but also forms that are not in association with counter ions (e.g., aqueous or organic solutions). Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound. In addition, salts may be formed from acid addition of certain organic and inorganic acids to basic centers, typically amines, or to acidic groups. Examples of such appropriate acids include, for instance, inorganic acids such as hydrohalogen acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like. Furthermore, this term also includes the solvates which the compounds of formulae herein as well as their salts are able to form, such as for example hydrates, alcoholates and the like. Finally, it is to be understood that the compositions herein comprise compounds of the invention in their unionized, as well as zwitterionic form, and combinations with stoichiometric amounts of water as in hydrates.

Also included within the scope of this invention are the salts of the parental compounds with one or more amino acids, especially the naturally-occurring amino acids found as protein components. The amino acid typically is one bearing a side chain with a basic or acidic group, e.g., lysine, arginine or glutamic acid, or a neutral group such as glycine, serine, threonine, alanine, isoleucine, or leucine.

The compounds of the invention also include physiologically acceptable salts thereof. Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX₄⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

As used herein and unless otherwise stated, the term “enantiomer” means each individual optically active form of a compound of the invention, having an optical purity or enantiomeric excess (as determined by methods standard in the art) of at least 80% (e.g. at least 90% of one enantiomer and at most 10% of the other enantiomer), preferably at least 90% and more preferably at least 98%.

The term “isomers” as used herein means all possible isomeric forms, including tautomeric and stereochemical forms, which the compounds of formulae herein may possess, but not including position isomers. Typically, the structures shown herein exemplify only one tautomeric or resonance form of the compounds, but the corresponding alternative configurations are contemplated as well. Unless otherwise stated, the chemical designation of compounds denotes the mixture of all possible stereochemically isomeric forms, said mixtures containing all diastereomers and enantiomers (since the compounds of formulae herein may have at least one chiral center) of the basic molecular structure, as well as the stereochemically pure or enriched compounds. More particularly, stereogenic centers may have either the R- or S-configuration, and multiple bonds may have either cis- or trans-configuration.

Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (e.g. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question.

Separation of stereoisomers is accomplished by standard methods known to those in the art. One enantiomer of a compound of the invention can be separated substantially free of its opposing enantiomer by a method such as formation of diastereomers using optically active resolving agents (“Stereochemistry of Carbon Compounds,” (1962) by E. L. Eliel, McGraw Hill; Lochmuller, C. H., (1975) J. Chromatogr., 113:(3) 283-302). Separation of isomers in a mixture can be accomplished by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure enantiomers, or (3) enantiomers can be separated directly under chiral conditions. Under method (1), diastereomeric salts can be formed by reaction of enantiomerically pure chiral bases such as brucine, quinine, ephedrine, strychnine, a-methyl-b-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts. Alternatively, by method (2), the substrate to be resolved may be reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched compound. A method of determining optical purity involves making chiral esters, such as a menthyl ester or Mosher ester, a-methoxy-a-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers can be separated and isolated by normal- and reverse-phase chromatography following methods for separation of atropisomeric naphthyl-isoquinolines (Hoye, T., WO 96/15111). Under method (3), a racemic mixture of two asymmetric enantiomers is separated by chromatography using a chiral stationary phase. Suitable chiral stationary phases are, for example, polysaccharides, in particular cellulose or amylose derivatives. Commercially available polysaccharide based chiral stationary phases are ChiralCel™ CA, OA, OB5, OC5, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like. (“Chiral Liquid Chromatography” (1989) W. J. Lough, Ed. Chapman and Hall, New York; Okamoto, (1990) “Optical resolution of dihydropyridine enantiomers by High-performance liquid chromatography using phenylcarbamates of polysaccharides as a chiral stationary phase”, J. of Chromatogr. 513:375-378).

The terms cis and trans are used herein in accordance with Chemical Abstracts nomenclature and include reference to the position of the substituents on a ring moiety. The absolute stereochemical configuration of the compounds of the formulae described herein may easily be determined by those skilled in the art while using well-known methods such as, for example, X-ray diffraction.

The compounds of the invention may be formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. Formulations optionally contain excipients such as those set forth in the “Handbook of Pharmaceutical Excipients” (1986) and include ascorbic acid and other antioxidants, chelating agents such as EDTA, carbohydrates such as dextrin, hydroxyalkylcellulose, hydroxyalkylmethylcellulose, stearic acid and the like.

Subsequently, the term “pharmaceutically acceptable carrier” as used herein means any material or substance with which the active ingredient is formulated in order to facilitate its application or dissemination to the locus to be treated, for instance by dissolving, dispersing or diffusing the said composition, and/or to facilitate its storage, transport or handling without impairing its effectiveness. The pharmaceutically acceptable carrier may be a solid or a liquid or a gas which has been compressed to form a liquid, e.g. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, e.g. carriers and additives which do not create permanent damage to mammals. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents. may also be prepared by micronisation, for instance in view to obtain them in the form of microspheres usually having a diameter of about 1 to 10 gm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable from coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcoholamine salts of dodecylbenzene sulphonic acid or dibutyl-naphthalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8C22 alkyl radical (e.g. cetyl, lauryl, palmityl, myristyl, ° leyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbucw’, 2 d ed. (Hanser Verlag, Vienna, 1981) and “Encyclopaedia of Surfactants, (Chemical Publishing Co., New York, 1981).

Compounds of the invention and their pharmaceutically acceptable salts (hereafter collectively referred to as the active ingredients) may be administered by any route appropriate to the condition to be treated, suitable routes including oral, rectal, nasal, topical (including ocular, buccal and sublingual), vaginal and parenteral (including subcutaneous, intramuscular, intravenous, intradermal, intrathecal and epidural). The preferred route of administration may vary with for example the condition of the recipient.

While it is possible for the active ingredients to be administered alone it is preferable to present them as pharmaceutical formulations. The formulations, both for veterinary and for human use, of the present invention comprise at least one active ingredient, as above described, together with one or more pharmaceutically acceptable carriers therefore and optionally other therapeutic ingredients. The carrier(s) optimally are “acceptable” in the sense of being compatible with the other ingredients of the formulation and not deleterious to the recipient thereof. The formulations include those suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein. For infections of the eye or other external tissues e.g. mouth and skin, the formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, e.g. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is optionally present in such formulations in a concentration of 0.5 to 20%, advantageously 0.5 to 10% particularly about 1.5% w/w. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate. Formulations suitable for nasal administration wherein the carrier is a solid include a coarse powder having a particle size for example in the range 20 to 500 microns (including particle sizes in a range between 20 and 500 microns in increments of 5 microns such as 30 microns, 35 microns, etc), which is administered in the manner in which snuff is taken, e.g. by rapid inhalation through the nasal passage from a container of the powder held close up to the nose. Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods.

Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethylcellulose, polymethyl methacrylate and the other above-described polymers. Such methods include colloid drug delivery systems like liposomes, microspheres, microemulsions, nanoparticles, nanocapsules and so on. Depending on the route of administration, the pharmaceutical composition may require protective coatings. Pharmaceutical forms suitable for injectionable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol and the like and mixtures thereof.

In view of the fact that, when several active ingredients are used in combination, they do not necessarily bring out their joint therapeutic effect directly at the same time in the mammal to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection or an aerosol.

Another embodiment of this invention relates to various precursor or “pro-drug” forms of the compounds of the present invention. It may be desirable to formulate the compounds of the present invention in the form of a chemical species which itself is not significantly biologically-active, but which when delivered to the animal, mammal or human will undergo a chemical reaction catalyzed by the normal function of the body of the fish, inter alia, enzymes present in the stomach or in blood serum, said chemical reaction having the effect of releasing a compound as defined herein. The term “pro-drug” thus relates to these species which are converted in vivo into the active pharmaceutical ingredient.

The pro-drugs of the compounds of the present invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

For the purpose of the present invention the term “therapeutically suitable pro-drug” is defined herein as “a compound modified in such a way as to be transformed in vivo to the therapeutically active form, whether by way of a single or by multiple biological transformations, when in contact with the tissues of the animal, mammal or human to which the pro-drug has been administered, and without undue toxicity, irritation, or allergic response, and achieving the intended therapeutic outcome ”.

More specifically the term “prodrug”, as used herein, relates to an inactive or significantly less active derivative of a compound such as represented by the structural formulae herein described, which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

The compounds of the invention can be prepared while using a series of chemical reactions well known to those skilled in the art, altogether making up the process for preparing said compounds and exemplified further. The processes described further are only meant as examples and by no means are meant to limit the scope of the present invention.

Abbreviations used in the instant specification, particularly in the schemes and examples, are as follows:

AcOH Acetic acid
DIPEA Diisopropyl-ethyl amine

### DME 1,2-Dimethoxyethane

### DMF N,N-Dimethylformamide

### DMSO Dimethylsulfoxide

DTBAD tert-Butylazodicarboxylate

### EtOH Ethanol

Eq. Equivalent

### h Hour

HATU O-(7-Azabenzotriazol-1-yl)-N,N,N′,N′-tetramethyluronium hexafluorophosphate
HPLC High performance liquid chromatography
LG Leaving group
min. Minute
NMP 1-Methyl-2-pyrrolidinone
Pd(PPh₃)₄ Tetrakis-(triphenylphosphine)-palladium(O)
Pd₂(dba)₃ Tris(dibenzylideneacetone)dipalladium

### PPh₃ Triphenylphospine

### TEA Triethylamine

TFA Trifluoroacetic acid

The compounds of interest having a structure according to the general formula (A) and all other formulas described herein and embodiments thereof can be prepared as outlined in the general chemical scheme 1.

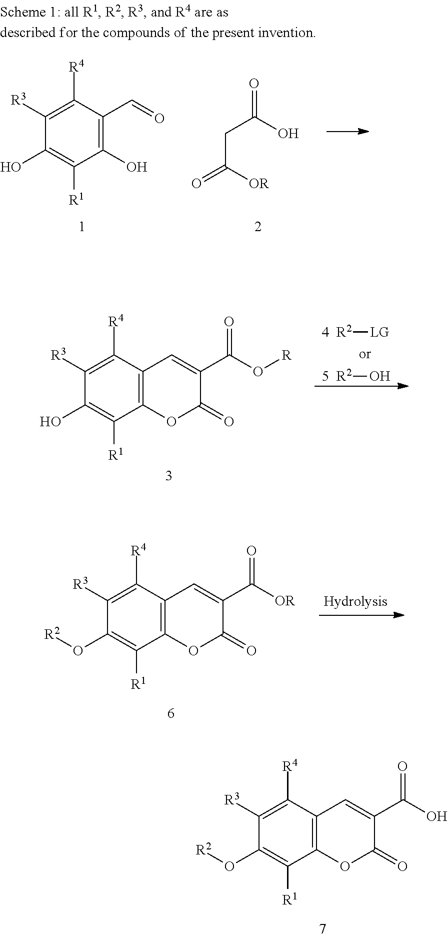

2,4-Dihydroxybenzaldehyde derivatives of formula 1 (commercially available or synthesized), may be condensed with a malonate ester of formula 2 (commercially available or synthesized by procedures known to the skilled in the art), wherein R is an ester protecting group (e.g. methyl, ethyl, t-Bu and the like), in the presence of an ammonium salt (e.g., tetramethymammonium hydroxide, ammonium chloride, and the like), with or without a catalytic amount of a base (e.g., piperidine, morpholine, and the like) in a polar solvent (e.g., acetonitrile, methanol, ethanol, and the like) at a temperature raising from 0 to 150° C. to provide compounds of formula 3. More detailed information can be found in the following references (Synth. Comm. 2005, 35(6), 785-790 and WO2012028709). Compounds of formula 3 may then be converted in the desired compounds of formula 6 via nucleophilic substitution using intermediates of formula 4 (commercially available or synthesized), wherein LG is a leaving group (preferably bromine), in the presence of a base (e.g., DIPEA, DBU, triethylamine, K₂CO₃, and the like) in a polar solvent (e.g., acetonitrile, DMF, NMP, and the like), with or without a chelating agent (e.g., 18-crown-6, cis-anti-cis-dicyclohexano-18-crown-6, and the like) at a temperature raising from 0 to 150° C. Alternatively, compounds of formula 3 may also be reacted with intermediates of formula 5 (commercially available or synthesized) in the presence of an azodicarboxylate reagent (e.g., diethylazodicarboxylate, tert-butylazodicarboxylate, and the like) and a phosphine (e.g., triphenylphosphine and the like) in a solvent (e.g., THF, toluene, and the like) at a temperature raising from 0 to 150° C., to provide the desired compounds of formula 6. Ester derivatives 6 may then be converted in the desired compounds of formula 7 via standard saponification reactions. Furthermore, in the different stages of the synthetic scheme, the required ester with R⁶ (as in formula (A)) can be obtained via standard transesterifications (starting from 6) or esterifications (starting from 7).

In a more particular embodiment, the compounds of the present invention may be synthesized as depicted in scheme 2.

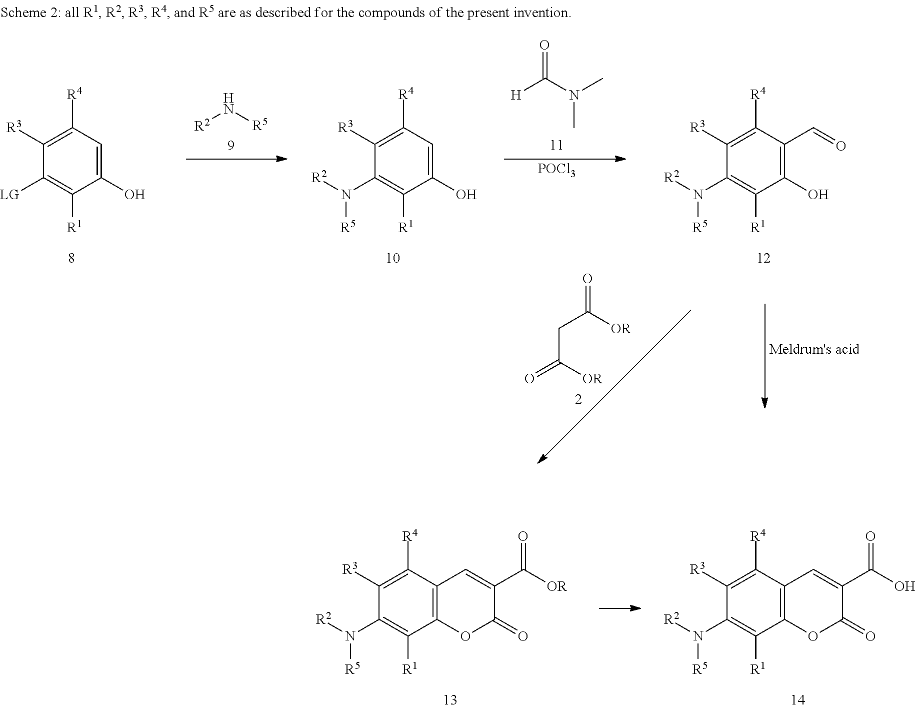

Phenol derivatives of formula 8 (commercially available or synthesized), wherein LG is a leaving group (preferably selected from Cl, Br, I or triflate), may be reacted with amines of formula 9, in the presence of a strong base (e.g., tBuOK, LiHMDS, and the like), a catalytic amount of palladium (e.g., Pd(OAc)₂, Pd₂(dba)₃, and the like) and a phosphine ligand (e.g., PPh₃, Verkade's base, and the like) in an aprotic solvent (e.g., toluene, THF, and the like) at a temperature raising from 25 to 150° C., to provide intermediates of general formula 10. More detailed information can be found in the following references (WO2009097144 and Adv. Synth. Cata. 2004, 346(6), 611-616). Intermediates 10 can be converted into intermediates of formula 12 via a Vilsmeier-Haack type reaction with DMF in the presence of phosphorus oxychloride at a temperature raising from 25 to 150° C. Intermediates of formula 12 may be condensed with a malonate ester of formula 2, wherein R is an ester protecting group (e.g. methyl, ethyl, t-Bu and the like), in the presence of an ammonium salt (e.g., tetramethymammonium hydroxide, ammonium chloride, and the like), with or without a catalytic amount of a base (e.g., piperidine, morpholine, and the like) in a polar solvent (e.g., acetonitrile, ethanol, and the like) at a temperature raising from 0 to 150° C. to provide compounds of formula 13, which can be converted into compounds of formula 14 following standard saponification reactions. Alternatively, intermediates of formula 12 may be converted directly into compounds of formula 14 by using meldrum's acid as known to the skilled in the art or as set forth in the examples below. Furthermore, in the different stages of the synthetic scheme, the required ester with R⁶ (as in formula (A)) can be obtained via standard transesterifications (starting from 13) or esterifications (starting from 14).

In a more particular embodiment, the compounds of the present invention may be synthesized as depicted in scheme 3

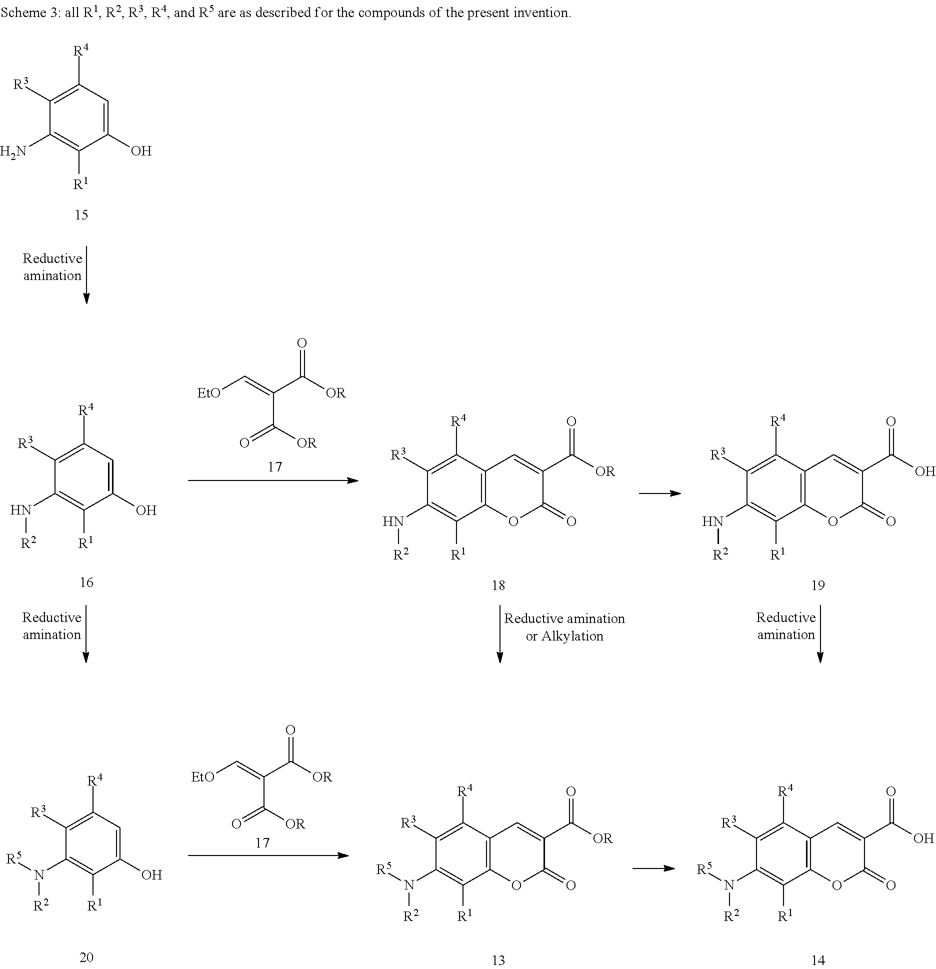

Derivatives of formula 15 (commercially available or synthesized) may be reacted with aldehydes or ketones, in the presence of a reducing agent (e.g., NaBH₃CN, NaBH₄, and the like) in a polar solvent (e.g., dichloromethane, ethanol, and the like) to furnish intermediates of formula 16. Intermediates of formula 16, may be condensed with derivatives of formula 17, wherein R is an ester protecting group (e.g. methyl, ethyl, t-Bu and the like), in the presence of a Lewis acid (e.g., TiCl₄, ZnCl₂, and the like) in a polar solvent (e.g., EtOH, THF, and the like) to provide compounds of formula 18, which can be converted into compounds of formula 19 following standard saponification conditions. Finally, compounds of formula 19 may be converted into compounds of formula 14 following reductive amination conditions known to the skilled in the art or as set forth in the examples below. Alternatively, intermediates 16 may be converted into intermediates of formula 20 via reductive amination conditions known to the skilled in the art or as set forth in the examples below. Intermediates of formula 20, may be condensed with derivatives of formula 17, wherein R is an ester protecting group (e.g. methyl, ethyl, t-Bu and the like), in the presence of a Lewis acid (e.g., TiCl₄, ZnCl₂, and the like) in a polar solvent (e.g., EtOH, THF, and the like) to provide compounds of formula 13, which can be converted into compounds of formula 14 following standard saponification conditions. Alternatively, compounds of formula 18 may be converted into compounds of formula 13 via reductive amination or alkylation reactions using halides of formula R⁶LG in the presence of a strong base (e.g., LiHMDS, NaH, and the like) in an aprotic solvent (e.g., THF, DMF, and the like) at a temperature raising from −78° C. to 150° C. Furthermore, in the different stages of the synthetic scheme, the required ester with R⁶ (as in formula (A)) can be obtained via standard transesterifications (starting from 18 or 13) or esterifications (starting from 19 or 14).

Compounds of formula 15 may be reacted with an acid chloride or a sulfonyl chloride in the presence of base (e.g., DIPEA, pyridine, and the like) in a polar solvent (e.g., dichloromethane, THF, and the like) to furnish compounds of formula 16 with HNR² being an amide (e.g. CONH, SO2NH) and can be further reacted as shown in scheme 3.

In a more particular embodiment, the compounds of the present invention may be synthesized as depicted in scheme 4.

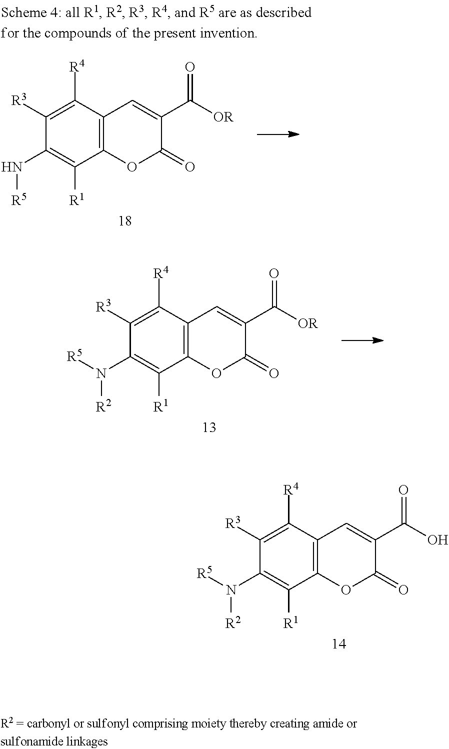

Compounds of formula 18 may be reacted with an acid chloride or a sulfonyl chloride in the presence of base (e.g., DIPEA, pyridine, and the like) in a polar solvent (e.g., dichloromethane, THF, and the like) to furnish compounds of formula 13, which may be converted into compounds of formula 14 following standard saponification conditions known to the skilled in the art or as set forth in the examples below. Furthermore, in the different stages of the synthetic scheme, the required ester with R⁶ (as in formula (A)) can be obtained via standard transesterifications (starting from 18 or 13) or esterifications (starting from 14).

### EXAMPLES

The following examples are provided for the purpose of illustrating the present invention and by no means should be interpreted to limit the scope of the present invention. Part A represents the preparation of the compounds whereas Part B represents the pharmacological examples.

TABLE 1
Structures of example compounds of the invention and their respective codes and names.

| Code | Structure | Name |
|---|---|---|
| cpd 1 | 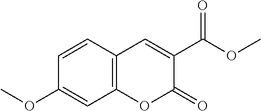 | methyl 7-methoxy-2-oxo-2H- chromene-3-carboxylate |
| cpd 2 | 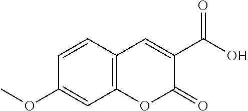 [SMILES: COc1ccc2cc(C(=O)O)c(=O)oc2c1] | 7-methoxy-2-oxo-2H- chromene-3-carboxylic acid |
| cpd 3 | 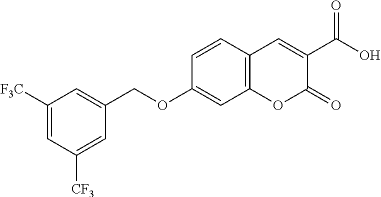 [SMILES: O=C(O)c1cc2ccc(OCc3cc(C(F)(F)F)cc(C(F)(F)F)c3)cc2oc1=O] | 7-((3,5- bis(trifluoromethyl)benzyl) oxy)-2-oxo-2H-chromene-3- carboxylic acid |
| cpd 4 | 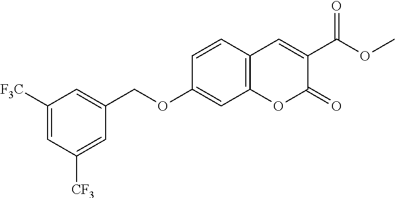 [SMILES: COC(=O)c1cc2ccc(OCc3cc(C(F)(F)F)cc(C(F)(F)F)c3)cc2oc1=O] | methyl 7-((3,5- bis(trifluoromethyl)benzyl) oxy)-2-oxo-2H-chromene-3- carboxylate |
| cpd 5 | 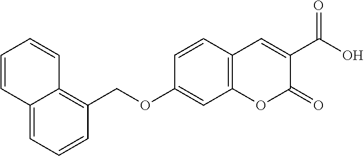 [SMILES: O=C(O)c1cc2ccc(OCc3cccc4ccccc34)cc2oc1=O] | 7-(naphthalen-1-ylmethoxy) 2-oxo-2H-chromene-3- carboxylic acid |
| cpd 6 | 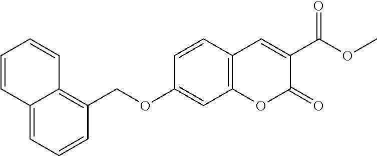 [SMILES: COC(=O)c1cc2ccc(OCc3cccc4ccccc34)cc2oc1=O] | methyl 7-(naphthalen-1- ylmethoxy)-2-oxo-2H- chromene-3-carboxylate |
| cpd 7 | 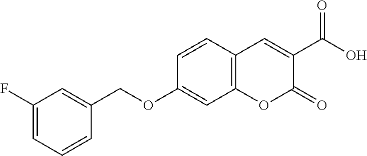 [SMILES: O=C(O)c1cc2ccc(OCc3cccc(F)c3)cc2oc1=O] | 7-((3-fluorobenzyl)oxy)-2- oxo-2H-chromene-3- carboxylic acid |
| cpd 8 | 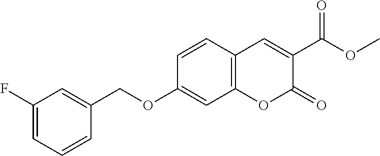 [SMILES: COC(=O)c1cc2ccc(OCc3cccc(F)c3)cc2oc1=O] | methyl 7-((3- fluorobenzyl)oxy)-2-oxo-2H- chromene-3-carboxylate |
| cpd 9 | 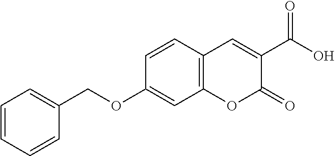 [SMILES: O=C(O)c1cc2ccc(OCc3ccccc3)cc2oc1=O] | 7-(benzyloxy)-2-oxo-2H- chromene-3-cartboxylic acid |
| cpd 10 | 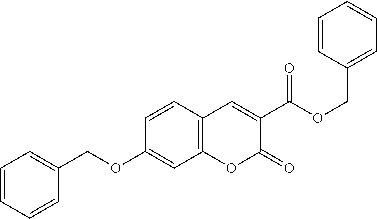 [SMILES: O=C(OCc1ccccc1)c1cc2ccc(OCc3ccccc3)cc2oc1=O] | benzyl 7-(benzyloxy)-2-oxo- 2H-chromene-3- carboxylate |
| cpd 11 | 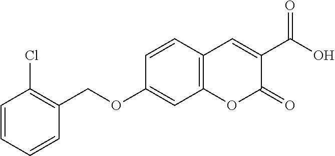 [SMILES: O=C(O)c1cc2ccc(OCc3ccccc3Cl)cc2oc1=O] | 7-((2-chlorobenzyl)oxy)-2- oxo-2H-chromene-3- carboxylic acid |
| cpd 12 | 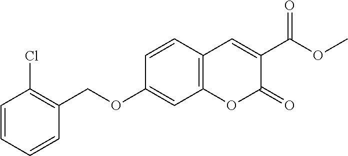 [SMILES: COC(=O)c1cc2ccc(OCc3ccccc3Cl)cc2oc1=O] | methyl 7-((2- chlorobenzyl)oxy)-2-oxo-2H- chromene-3-carboxylate |
| cpd 13 | 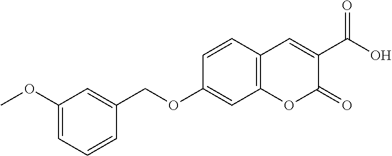 [SMILES: COc1cccc(COc2ccc3cc(C(=O)O)c(=O)oc3c2)c1] | 7-((3-methoxybenzyl)oxy)-2- oxo)-2H-chromene-3- carboxylic acid |
| cpd 14 | 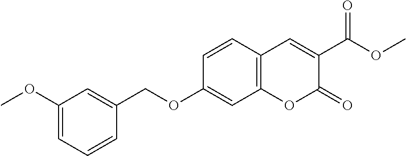 | methyl 7-((3- methoxybenzyl)oxy)-2-oxo- 2H-chromene-3-carboxylate |
| cpd 15 | 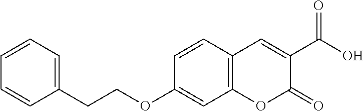 [SMILES: O=C(O)c1cc2ccc(OCCc3ccccc3)cc2oc1=O] | 2-oxo-7-phenethoxy-2H- chromene-3-carboxylic acid |
| cpd 16 | 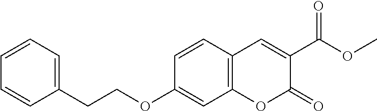 [SMILES: COC(=O)c1cc2ccc(OCCc3ccccc3)cc2oc1=O] | methyl 2-oxo-7-phenethoxy- 2H-chromene-3-carboxylate |
| cpd 17 | 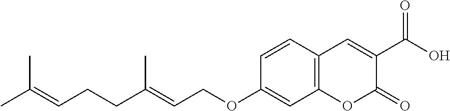 [SMILES: CC(C)=CCC/C(C)=C/COc1ccc2cc(C(=O)O)c(=O)oc2c1] | (E)-7-((3,7-dimethylocta-2,6- dien-1-yl)oxy)-2-oxo-2H- chromene-3-carboxylic acid |
| cpd 18 | 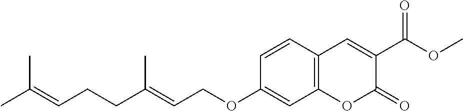 [SMILES: COC(=O)c1cc2ccc(OC/C=C(\C)CCC=C(C)C)cc2oc1=O] | (E)-methyl 7-((3,7- dimethylocta-2,6-dien-1- yl)oxy)-2-oxo-2H-chromene- 3-carboxylate |
| cpd 19 | 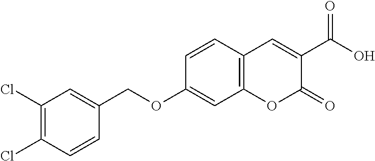 [SMILES: O=C(O)c1cc2ccc(OCc3ccc(Cl)c(Cl)c3)cc2oc1=O] | 7-((3,4-dichlorobenzyl)oxy)-2- oxo-2H-chromene-3- carboxylic acid |
| cpd 20 | 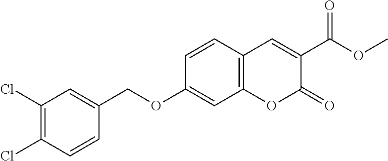 [SMILES: COC(=O)c1cc2ccc(OCc3ccc(Cl)c(Cl)c3)cc2oc1=O] | methyl 7-((3,4- dichlorobenzyl)oxy)-2-oxo- 2H-chromene-3-carboxylate |
| cpd 21 | 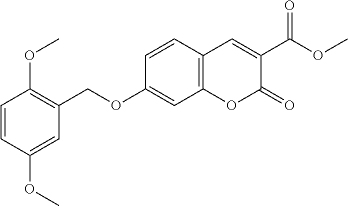 [SMILES: COC(=O)c1cc2ccc(OCc3cc(OC)ccc3OC)cc2oc1=O] | methyl 7-((2,5- dimethoxybenzyl)oxy)-2-oxo- 2H-chromene-3- carboxylate |
| cpd 22 | 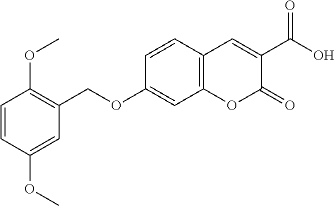 [SMILES: COc1ccc(OC)c(COc2ccc3cc(C(=O)O)c(=O)oc3c2)c1] | 7-(2,5- dimethoxybenzyl)oxy)-2-oxo- 2H-chromene-3-carboxylic acid |
| cpd 23 | 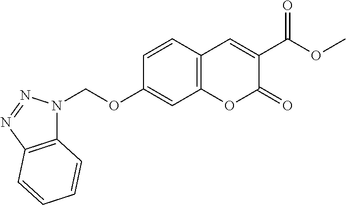 [SMILES: COC(=O)c1cc2ccc(OCn3nnc4ccccc43)cc2oc1=O] | methyl 7-((1H- benzo[d][1,2,3]triazol-1- yl)methoxy)-2-oxo-2H- chromene-3-carboxylate |
| cpd 24 | 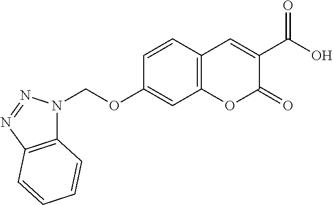 [SMILES: O=C(O)c1cc2ccc(OCn3nnc4ccccc43)cc2oc1=O] | 7-((1H- benzo[d][1,2,3]triazol-1- yl)methoxy)-2-oxo-2H- chromene-3-carboxylic acid |
| cpd 25 | 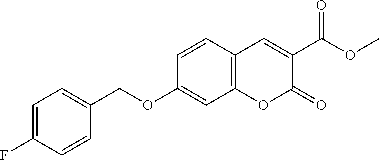 [SMILES: COC(=O)c1cc2ccc(OCc3ccc(F)cc3)cc2oc1=O] | methyl 7-((4- fluorobenzyl)oxy)-2-oxo-2H- chromene-3-carboxylate |
| cpd 26 | 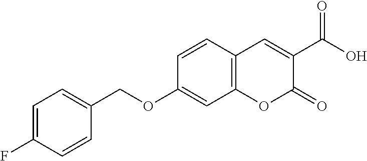 [SMILES: O=C(O)c1cc2ccc(OCc3ccc(F)cc3)cc2oc1=O] | 7-((4-fluorobenzyl)oxy)-2- oxo-2H-chromene-3- carboxylic acid |
| cpd 27 | 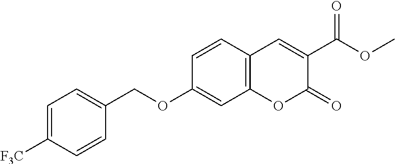 [SMILES: COC(=O)c1cc2ccc(OCc3ccc(C(F)(F)F)cc3)cc2oc1=O] | methyl 2-oxo-7-((4- (trifluoromethyl)benzyl)oxy)- 2H-chromene-3- carboxylate |
| cpd 28 | 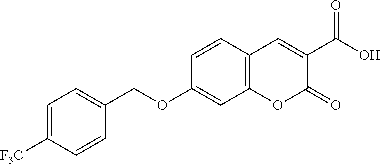 [SMILES: O=C(O)c1cc2ccc(OCc3ccc(C(F)(F)F)cc3)cc2oc1=O] | 2-oxo-7-((4- (trifluoromethyl)benzyl)oxy)- 2H-chromene-3-carboxylic acid |
| cpd 29 | 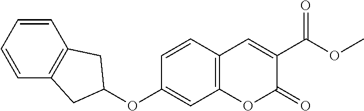 [SMILES: COC(=O)c1cc2ccc(OC3Cc4ccccc4C3)cc2oc1=O] | methyl 7-((2,3-dihydro-1H- inden-2-yl)oxy)-2-oxo-2H- chromene-3-carboxylate |
| cpd 30 | 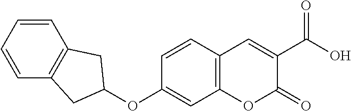 [SMILES: O=C(O)c1cc2ccc(OC3Cc4ccccc4C3)cc2oc1=O] | 7-((2,3-dihydro-1H-inden-2- yl)oxy)-2-oxo-2H-chromene- 3-carboxylic acid |
| cpd 31 | 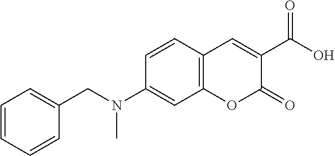 [SMILES: CN(Cc1ccccc1)c1ccc2cc(C(=O)O)c(=O)oc2c1] | 7-(benzyl(methyl)amino)-2- oxo-2H-chromene-3- carboxylic acid |
| cpd 32 | 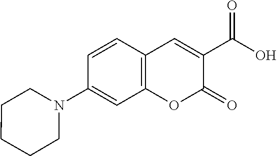 [SMILES: O=C(O)c1cc2ccc(N3CCCCC3)cc2oc1=O] | 2-oxo-7-(piperidin-1-yl)-2H- chromene-3-carboxylic acid |
| cpd 33 | 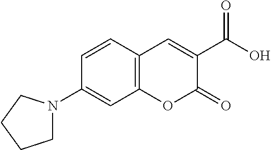 [SMILES: O=C(O)c1cc2ccc(N3CCCC3)cc2oc1=O] | 2-oxo-7-(pyrrolidin-1-yl)-2H- chromene-3-carboxylic acid |
| cpd 34 | 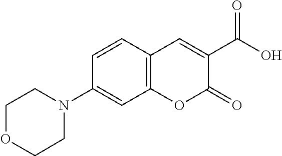 [SMILES: O=C(O)c1cc2ccc(N3CCOCC3)cc2oc1=O] | 7-morpholino-2-oxo-2H- chromene-3-carboxylic acid |
| cpd 35 | 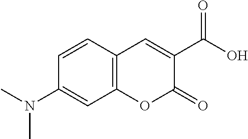 [SMILES: CN(C)c1ccc2cc(C(=O)O)c(=O)oc2c1] | 7-(dimethlamino)-2-oxo-2H- chromene-3-carboxylic acid |
| cpd 36 | 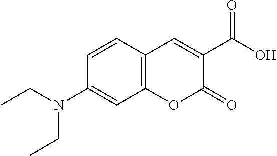 [SMILES: CCN(CC)c1ccc2cc(C(=O)O)c(=O)oc2c1] | 7-(diethylamino)-2-oxo-2H- chromene-3-carboxylic acid |
| cpd 37 | 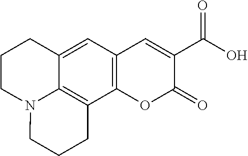 [SMILES: O=C(O)c1cc2cc3c4c(c2oc1=O)CCCN4CCC3] | 11-oxo-2,3,5,6,7,11- hexahydro-1H-pyrano[2,3 - f]pyrido[3,2,1-ij]quinoline-10- carboxylic acid |
| cpd 38 | 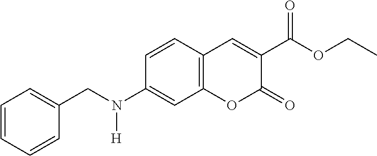 [SMILES: CCOC(=O)c1cc2ccc(NCc3ccccc3)cc2oc1=O] | ethyl 7-(benzylamino)-2-oxo- 2H-chromene-3-carboxylate |
| cpd 39 | 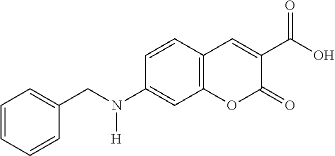 [SMILES: O=C(O)c1cc2ccc(NCc3ccccc3)cc2oc1=O] | 7-(benzylamino)-2-oxo-2H- chromene-3-carboxylic acid |
| cpd 40 | 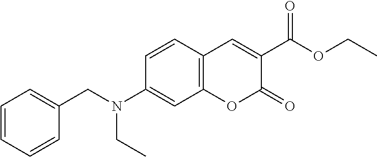 [SMILES: CCOC(=O)c1cc2ccc(N(CC)Cc3ccccc3)cc2oc1=O] | ethyl 7-(benzyl(ethyl)amino)- 2-oxo-2H-chromene-3- carboxylate |
| cpd 41 | 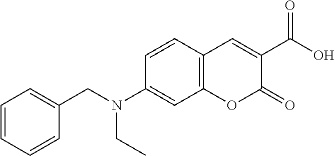 [SMILES: CCN(Cc1ccccc1)c1ccc2cc(C(=O)O)c(=O)oc2c1] | 7-(benzyl(ethyl)amino)-2- oxo-2H-chromene-3- carboxylic acid |
| cpd 42 | 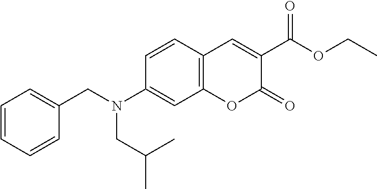 [SMILES: CCOC(=O)c1cc2ccc(N(Cc3ccccc3)CC(C)C)cc2oc1=O] | ethyl 7- (benzyl(isobutyl)amino)-2- oxo-2H-chromene-3- carboxylate |
| cpd 43 | 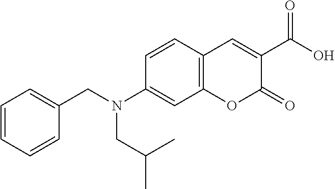 [SMILES: CC(C)CN(Cc1ccccc1)c1ccc2cc(C(=O)O)c(=O)oc2c1] | 7-(benzyl(isobutyl)amino)- 2-oxo-2H-chromene-3- carboxylic acid |
| cpd 44 | 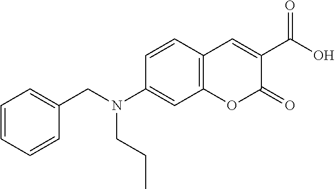 [SMILES: CCCN(Cc1ccccc1)c1ccc2cc(C(=O)O)c(=O)oc2c1] | 7-(benzyl(propyl)amino)-2- oxo-2H-chromene-3- carboxylic acid |
| cpd 45 | 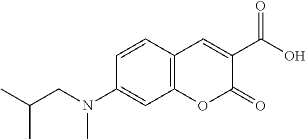 [SMILES: CC(C)CN(C)c1ccc2cc(C(=O)O)c(=O)oc2c1] | 7-(isobutyl(methyl)amino)- 2-oxo-2H-chromene-3- carboxylic acid |
| cpd 46 | 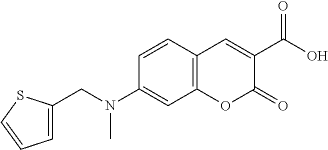 [SMILES: CN(Cc1cccs1)c1ccc2cc(C(=O)O)c(=O)oc2c1] | 7-(methyl(thiophen-2- ylmethyl)amino)-2-oxo-2H- chromene-3-carboxylic acid |
| cpd 47 | 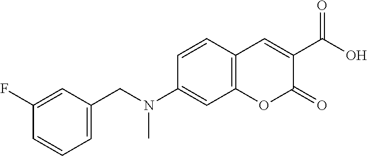 [SMILES: CN(Cc1cccc(F)c1)c1ccc2cc(C(=O)O)c(=O)oc2c1] | 7-((3- fluorobenzyl)(methyl)amino)- 2-oxo-2H-chromene-3- carboxylic acid |
| cpd 48 | 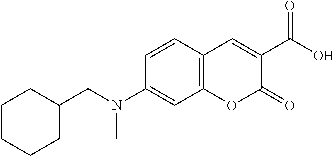 [SMILES: CN(CC1CCCCC1)c1ccc2cc(C(=O)O)c(=O)oc2c1] | 7- ((cyclohexylmethyl)(methyl) amino)-2-oxo-2H- chromene-3-carboxylic acid |
| cpd 49 | 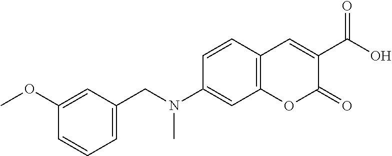 [SMILES: COc1cccc(CN(C)c2ccc3cc(C(=O)O)c(=O)oc3c2)c1] | 7-((3- methoxybenzyl)(methyl) amino)-2-oxo-2H-chromene-3- carboxylic acid |
| cpd 50 | 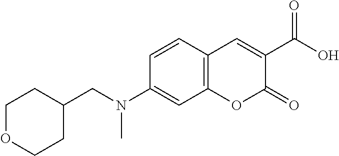 [SMILES: CN(CC1CCOCC1)c1ccc2cc(C(=O)O)c(=O)oc2c1] | 7-(methyl((tetrahydro-2H- pyran-4-yl)methyl)amino)- 2-oxo-2H-chromene-3- carboxylic acid |
| cpd 51 | 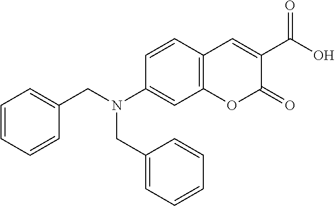 [SMILES: O=C(O)c1cc2ccc(N(Cc3ccccc3)Cc3ccccc3)cc2oc1=O] | 7-(dibenzylamino)-2-oxo-2H- chromene-3-carboxylic acid |
| cpd 52 | 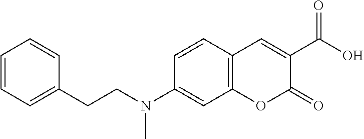 [SMILES: CN(CCc1ccccc1)c1ccc2cc(C(=O)O)c(=O)oc2c1] | 7-(methyl(phenethyl)amino)- 2-oxo-2H-chromene-3- carboxylic acid |
| cpd 53 | 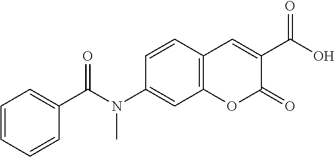 [SMILES: CN(C(=O)c1ccccc1)c1ccc2cc(C(=O)O)c(=O)oc2c1] | 7-(N-methylbenzamido)-2- oxo-2H-chromene-3- carboxylic acid |
| cpd 54 | 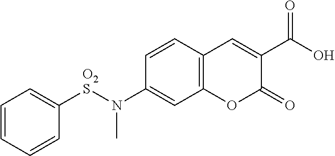 [SMILES: CN(c1ccc2cc(C(=O)O)c(=O)oc2c1)S(=O)(=O)c1ccccc1] | 7-(N- methylphenylsulfonamido)-2- oxo-2H-chromene-3- carboxylic acid |
| cpd 55 | 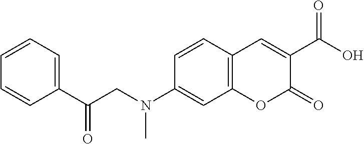 [SMILES: CN(CC(=O)c1ccccc1)c1ccc2cc(C(=O)O)c(=O)oc2c1] | 7-(methyl(2-oxo-2- phenytethyl)amino)-2-oxo- 2H-chromene-3-carboxylic acid |
| cpd 56 | 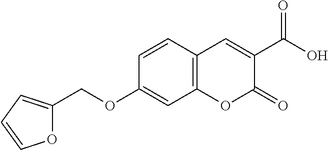 [SMILES: O=C(O)c1cc2ccc(OCc3ccco3)cc2oc1=O] | 7-(furan-2-ylmethoxy)-2- oxo-2H-chromene-3- carboxylic acid |
| cpd 57 | 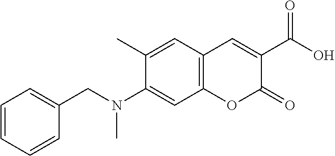 [SMILES: Cc1cc2cc(C(=O)O)c(=O)oc2cc1N(C)Cc1ccccc1] | 7-(benzyl(methyl)amino)-6- methyl-2-oxo-2H-chromene- 3-carboxylic acid |
| cpd 58 | 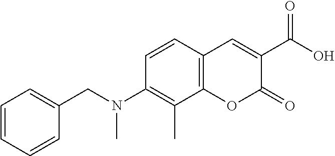 [SMILES: Cc1c(N(C)Cc2ccccc2)ccc2cc(C(=O)O)c(=O)oc12] | 7-(benzyl(methyl)amino)-8- methyl-2-oxo-2H-chromene- 3-carboxylic acid |
| cpd 59 | 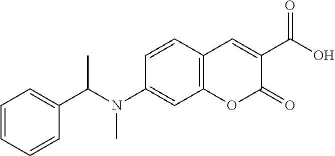 [SMILES: CC(c1ccccc1)N(C)c1ccc2cc(C(=O)O)c(=O)oc2c1] | 7-(methyl(1- phenytethyl)amino)-2-oxo- 2H-chromene-3-carboxylic acid |
| cpd 60 | 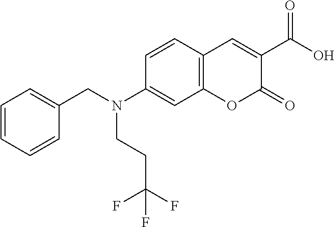 [SMILES: O=C(O)c1cc2ccc(N(CCC(F)(F)F)Cc3ccccc3)cc2oc1=O] | 7-(benzyl(3,3,3- trifluoropropyl)amino)-2-oxo- 2H-chromene-3-carboxylic acid |
| cpd 61 | 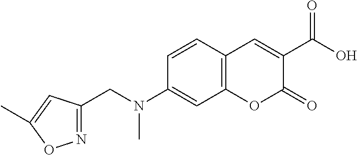 [SMILES: Cc1cc(CN(C)c2ccc3cc(C(=O)O)c(=O)oc3c2)no1] | 7-(methyl((5-methylisoxazol- 3-yl)methyl)amino)-2-oxo- 2H-chromene-3-carboxylic acid |
| cpd 62 | 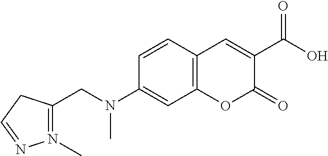 | 7-(methyl((1-methyl-1H- pyrazol-5-yl)methyl)amino)- 2-oxo-2H-chromene-3- carboxylic acid |
| cpd 63 | 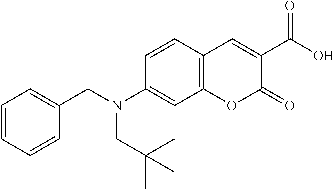 [SMILES: CC(C)(C)CN(Cc1ccccc1)c1ccc2cc(C(=O)O)c(=O)oc2c1] | 7-(benzyl(neopentyl)amino)- 2-oxo-2H-chromene-3- carboxylic acid |
| cpd 64 | 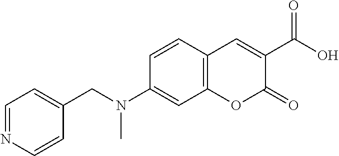 [SMILES: CN(Cc1ccncc1)c1ccc2cc(C(=O)O)c(=O)oc2c1] | 7-(methyl(pyridin-4- ylmethyl)amino)-2-oxo-2H- chromene-3-carboxylic acid |
| cpd 65 | 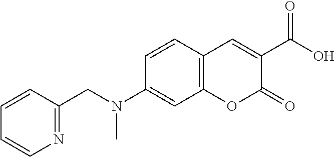 [SMILES: CN(Cc1ccccn1)c1ccc2cc(C(=O)O)c(=O)oc2c1] | 7-(methyl(pyridin-2- ylmethyl)amino)-2-oxo-2H- chromene-3-carboxylic acid |
| cpd 66 | 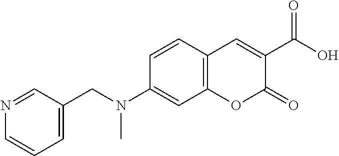 [SMILES: CN(Cc1cccnc1)c1ccc2cc(C(=O)O)c(=O)oc2c1] | 7-(methyl(pyridin-3- ylmethyl)amino)-2-oxo-2H- chromene-3-carboxylic acid |
| cpd 67 | 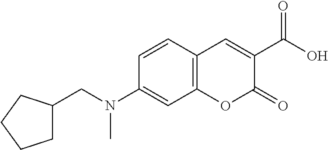 [SMILES: CN(CC1CCCC1)c1ccc2cc(C(=O)O)c(=O)oc2c1] | 7- ((cyclopentylmethyl)(methyl) amino)-2-oxo-2H- chromene-3-carboxylic acid |
| cpd 68 | 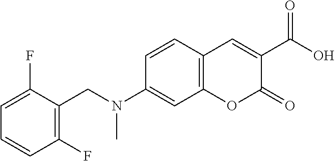 [SMILES: CN(Cc1c(F)cccc1F)c1ccc2cc(C(=O)O)c(=O)oc2c1] | 7-(2,6- difluorobenzyl)(methyl)amino)- 2-oxo-2H-chromene-3- carboxylic acid |
| cpd 69 | 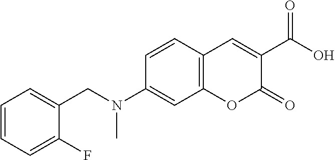 [SMILES: CN(Cc1ccccc1F)c1ccc2cc(C(=O)O)c(=O)oc2c1] | 7-((2- fluorobenzyl)(methyl)amino)- 2-oxo-2H-chromene-3- carboxylic acid |
| cpd 70 | 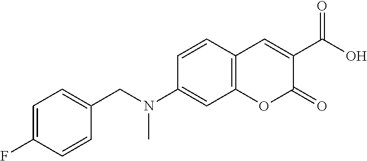 [SMILES: CN(Cc1ccc(F)cc1)c1ccc2cc(C(=O)O)c(=O)oc2c1] | 7-((4- fluorobenzyl)(methyl)amino)- 2-oxo-2H-chromene-3- carboxylic acid |
| cpd 71 | 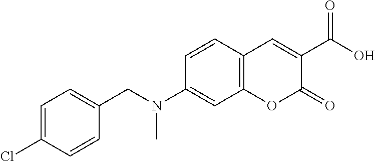 [SMILES: CN(Cc1ccc(Cl)cc1)c1ccc2cc(C(=O)O)c(=O)oc2c1] | 7-((4- chlorobenzyl)(methyl)amino)- 2-oxo-2H-chromene-3- carboxylic acid |
| cpd 72 | 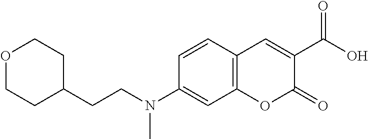 [SMILES: CN(CCC1CCOCC1)c1ccc2cc(C(=O)O)c(=O)oc2c1] | 7-(methyl(2-(tetrahydro-2H- pyran-4-yl)ethyl)amino)-2- oxo-2H-chromene-3- carboxylic acid |
| cpd 73 | 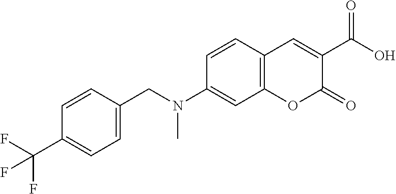 [SMILES: CN(Cc1ccc(C(F)(F)F)cc1)c1ccc2cc(C(=O)O)c(=O)oc2c1] | 7-(methyl(4- (trifluoromethyl)benzyl)amino)- 2-oxo-2H-chromene-3- carboxylic acid |
| cpd 74 | 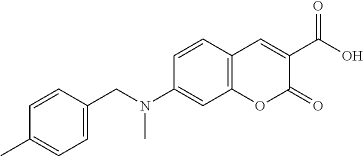 [SMILES: Cc1ccc(CN(C)c2ccc3cc(C(=O)O)c(=O)oc3c2)cc1] | 7-(methyl(4- methylbenzyl)amino)-2-oxo- 2H-chromene-3-carboxylic acid |
| cpd 75 | 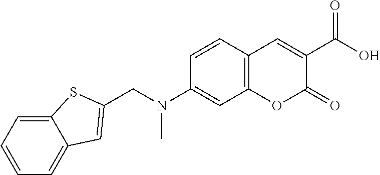 [SMILES: CN(Cc1cc2ccccc2s1)c1ccc2cc(C(=O)O)c(=O)oc2c1] | 7-((benzo[b]thiophen-2- ylmethyl)(methyl)amino)-2- oxo-2H-chromene-3- carboxylic acid |
| cpd 76 | 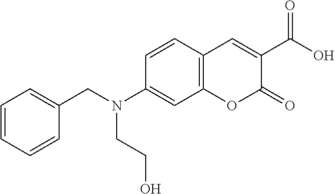 [SMILES: O=C(O)c1cc2ccc(N(CCO)Cc3ccccc3)cc2oc1=O] | 7-(benzyl(2- hydroxyethyl)amino)-2-oxo- 2H-chromene-3-carboxylic acid |
| cpd 77 | 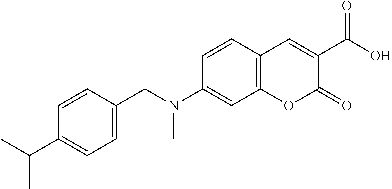 [SMILES: CC(C)c1ccc(CN(C)c2ccc3cc(C(=O)O)c(=O)oc3c2)cc1] | 7-((4- isopropylbenzyl)(methyl) amino)-2-oxo-2H-chromene-3- carboxylic acid |
| cpd 78 | 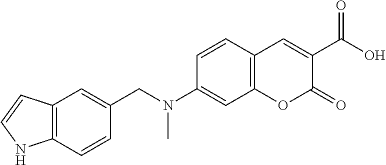 [SMILES: CN(Cc1ccc2[nH]ccc2c1)c1ccc2cc(C(=O)O)c(=O)oc2c1] | 7-(((1H-indol-5- yl)methyl)(methyl)amino)-2- oxo-2H-chromene-3- carboxylic acid |
| cpd 79 | 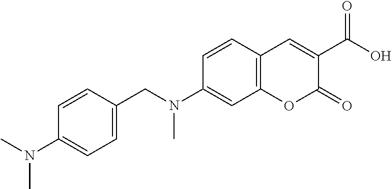 [SMILES: CN(C)c1ccc(CN(C)c2ccc3cc(C(=O)O)c(=O)oc3c2)cc1] | 7-((4- (dimethylamino)benzyl) (methyl)amino)-2-oxo-2H- chromene-3-carboxylic acid |
| cpd 80 | 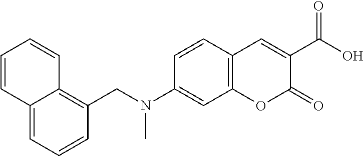 [SMILES: CN(Cc1cccc2ccccc12)c1ccc2cc(C(=O)O)c(=O)oc2c1] | 7-(methyl(naphthalen-1- ylmethyl)amino)-2-oxo-2H- chromene-3-carboxylic acid |
| cpd 81 | 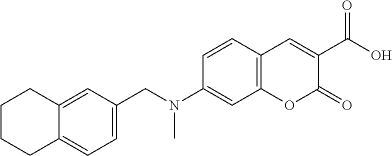 [SMILES: CN(Cc1ccc2c(c1)CCCC2)c1ccc2cc(C(=O)O)c(=O)oc2c1] | 7-(methyl((5,6,7,8- tetrahydronaphthalen-2- yl)methyl)amino)-2-oxo-2H- chromene-3-carboxylic acid |
| cpd 82 | 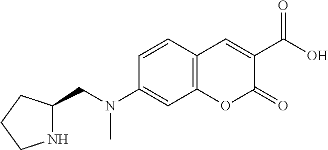 [SMILES: CN(C[C@@H]1CCCN1)c1ccc2cc(C(=O)O)c(=O)oc2c1] | (S)-7-(methyl(pyrrolidin-2- ylmethyl)amino)-2-oxo-2H- chromene-3-carboxylic acid |
| cpd 83 | 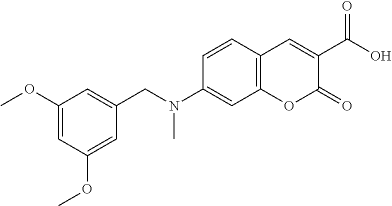 [SMILES: COc1cc(CN(C)c2ccc3cc(C(=O)O)c(=O)oc3c2)cc(OC)c1] | 7-((3,5- dimethoxybenzyl)(methyl) amino)-2-oxo-2H-chromene- 3-carboxylic acid |
| cpd 84 | 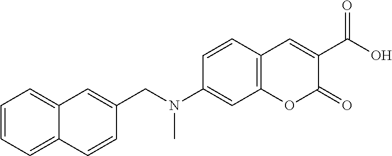 [SMILES: CN(Cc1ccc2ccccc2c1)c1ccc2cc(C(=O)O)c(=O)oc2c1] | 7-(methyl(quinolin-3- ylmethyl)amino)-2-oxo-2H- chromene-3-carboxylic acid |
| cpd 85 | 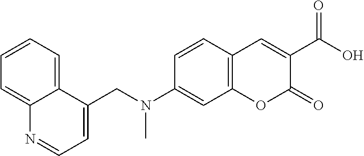 [SMILES: CN(Cc1ccnc2ccccc12)c1ccc2cc(C(=O)O)c(=O)oc2c1] | 7-(methyl(quinolin-4- ylmethyl)amino)-2-oxo-2H- chromene-3-carboxylic acid |
| cpd 86 | 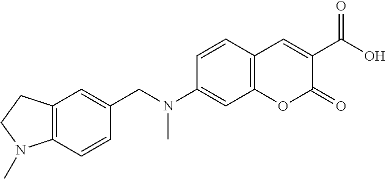 [SMILES: CN(Cc1ccc2c(c1)CCN2C)c1ccc2cc(C(=O)O)c(=O)oc2c1] | 7-(methyl((1-methylindolin- 5-yl)methyl)amino)-2-oxo- 2H-chromene-3-carboxylic acid |
| cpd 87 | 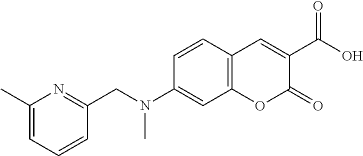 [SMILES: Cc1cccc(CN(C)c2ccc3cc(C(=O)O)c(=O)oc3c2)n1] | 7-(methyl((6-methylpyridin- 2-yl(methyl)amino)-2-oxo- 2H-chromene-3-carboxylic acid |
| cpd 88 | 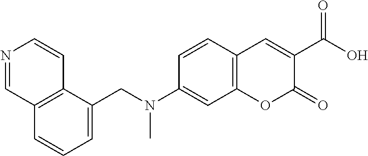 [SMILES: CN(Cc1cccc2cnccc12)c1ccc2cc(C(=O)O)c(=O)oc2c1] | 7-((isoquinolin-5- ylmethyl)(methyl)amino)-2- oxo-2H-chromene-3- carboxylic acid |
| cpd 89 | 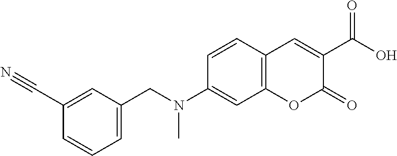 [SMILES: CN(Cc1cccc(C#N)c1)c1ccc2cc(C(=O)O)c(=O)oc2c1] | 7-((3- cyanobenzyl)(methyl)amino)- 2-oxo-2H-chromene-3- carboxylic acid |
| cpd 90 | 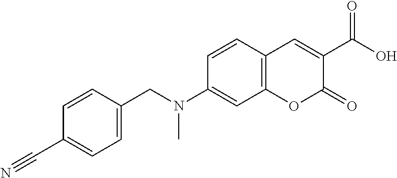 [SMILES: CN(Cc1ccc(C#N)cc1)c1ccc2cc(C(=O)O)c(=O)oc2c1] | 7-((4- cyanobenzyl)(methyl)amino)- 2-oxo-2H-chromene-3- carboxylic acid |
| cpd 91 | 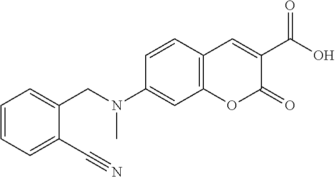 [SMILES: CN(Cc1ccccc1C#N)c1ccc2cc(C(=O)O)c(=O)oc2c1] | 7-((2- cyanobenzyl)(methyl)amino)- 2-oxo-2H-chromene-3- carboxylic acid |
| cpd 92 | 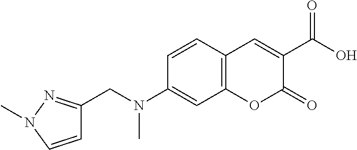 [SMILES: CN(Cc1ccn(C)n1)c1ccc2cc(C(=O)O)c(=O)oc2c1] | 7-(methyl((1-methyl-1H- pyrazol-3-yl)methyl)amino)-2- oxo-2H-chromene-3- carboxylic acid |
| cpd 93 | 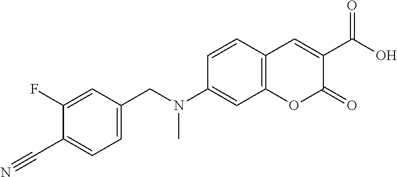 [SMILES: CN(Cc1ccc(C#N)c(F)c1)c1ccc2cc(C(=O)O)c(=O)oc2c1] | 7-((4-cyano-3- fluorobenzyl)(methyl)amino)- 2-oxo-2H-chromene-3- carboxylic acid |
| cpd 94 | 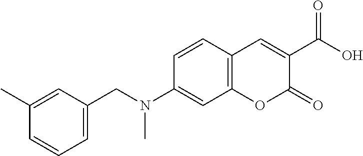 [SMILES: Cc1cccc(CN(C)c2ccc3cc(C(=O)O)c(=O)oc3c2)c1] | 7-(methyl(3- methylbenzyl)amino)-2-oxo- 2H-chromene-3-carboxylic acid |
| cpd 95 | 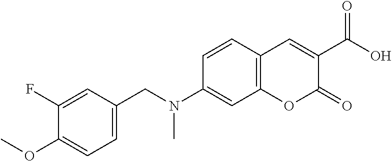 [SMILES: COc1ccc(CN(C)c2ccc3cc(C(=O)O)c(=O)oc3c2)cc1F] | 7-((3-fluoro-4- methoxybenzyl)(methyl)amino)- 2-oxo-2H-chromene-3- carboxylic acid |
| cpd 96 | 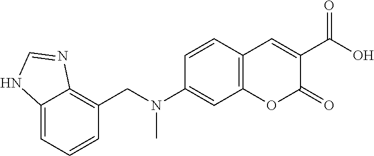 [SMILES: CN(Cc1cccc2[nH]cnc12)c1ccc2cc(C(=O)O)c(=O)oc2c1] | 7-(((1H-benzo[d]imidazol-4- yl)methyl)(methyl)amino)-2- oxo-2H-chromene-3- carboxylic acid |
| cpd 97 | 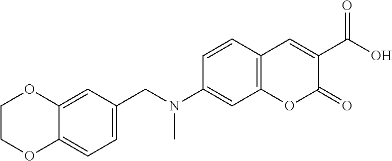 [SMILES: CN(Cc1ccc2c(c1)OCCO2)c1ccc2cc(C(=O)O)c(=O)oc2c1] | 7-(((2,3- dihydrobenzo[b][1,4]dioxin-6- yl)methyl)(methyl)amino)-2- oxo-2H-chromene-3- carboxylic acid |
| cpd 98 | 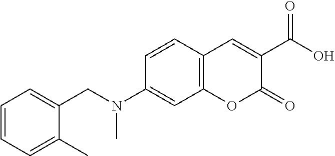 [SMILES: Cc1ccccc1CN(C)c1ccc2cc(C(=O)O)c(=O)oc2c1] | 7-(methyl(2- methylbenzyl)amino)-2-oxo- 2H-chromene-3-carboxylic acid |
| cpd 99 | 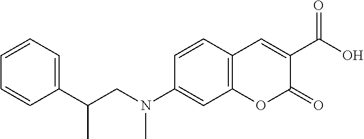 [SMILES: CC(CN(C)c1ccc2cc(C(=O)O)c(=O)oc2c1)c1ccccc1] | 7-(methyl(2- phenylpropyl)amino)-2-oxo- 2H-chromene-3-carboxylic acid |
| cpd 100 | 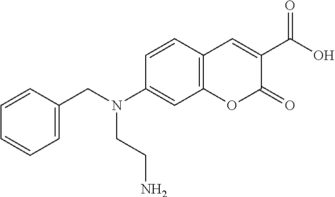 [SMILES: NCCN(Cc1ccccc1)c1ccc2cc(C(=O)O)c(=O)oc2c1] | 7-((2- aminoethyl)(benzyl)amino)-2- oxo-2H-chromene-3- carboxylic acid |
| cpd 101 | 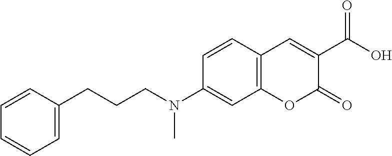 [SMILES: CN(CCCc1ccccc1)c1ccc2cc(C(=O)O)c(=O)oc2c1] | 7-(methyl(3- phenylpropyl)amino)-2-oxo- 2H-chromene-3-carboxylic acid |
| cpd 102 | 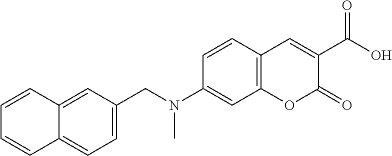 [SMILES: CN(Cc1ccc2ccccc2c1)c1ccc2cc(C(=O)O)c(=O)oc2c1] | 7-(methyl(naphthalen-2- ylmethyl)amino)-2-oxo-2H- chromene-3-carboxylic acid |
| cpd 103 | 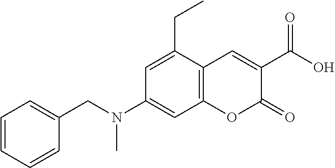 [SMILES: CCc1cc(N(C)Cc2ccccc2)cc2oc(=O)c(C(=O)O)cc12] | 7-(benzyl(methyl)amino)-5- ethyl-2-oxo-2H-chromene- 3-carboxylic acid |
| cpd 104 | 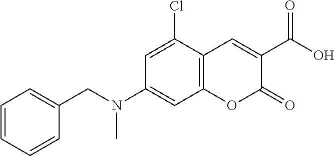 [SMILES: CN(Cc1ccccc1)c1cc(Cl)c2cc(C(=O)O)c(=O)oc2c1] | 7-(benzyl(methyl)amino)-5- chloro-2-oxo-2H-chromene- 3-carboxylic acid |
| cpd 105 | 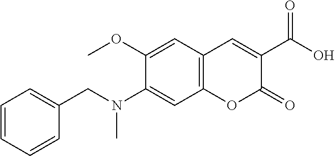 [SMILES: COc1cc2cc(C(=O)O)c(=O)oc2cc1N(C)Cc1ccccc1] | 7-(benzyl(methyl)amino)-6- methoxy-2-oxo-2H- chromene-3-carboxylic acid |
| cpd 106 | 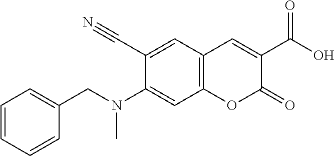 [SMILES: CN(Cc1ccccc1)c1cc2oc(=O)c(C(=O)O)cc2cc1C#N] | 7-(benzyl(methyl)amino)-6- cyano-2-oxo-2H-chromene 3-carboxylic acid |
| cpd 107 | 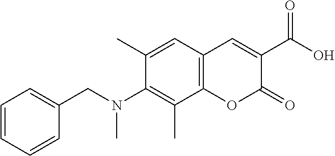 [SMILES: Cc1cc2cc(C(=O)O)c(=O)oc2c(C)c1N(C)Cc1ccccc1] | 7-(benzyl(methyl)amino)- 6,8-dimethyl-2-oxo-2H- chromene-3-carboxylic acid |
| cpd 108 | 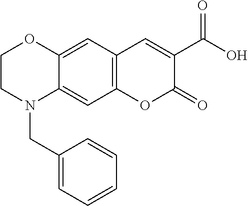 [SMILES: O=C(O)c1cc2cc3c(cc2oc1=O)N(Cc1ccccc1)CCO3] | 4-benzyl-7-oxo-2,3,4,7- tetrahydrochromeno[6,7- b][1,4]oxazine-8-carboxylic acid |
| cpd 109 | 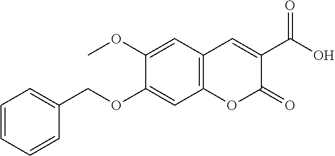 [SMILES: COc1cc2cc(C(=O)O)c(=O)oc2cc1OCc1ccccc1] | 7-(benzyloxy)-6-methoxy-2- oxo-2H-chromene-3- carboxylic acid |
| cpd 110 | 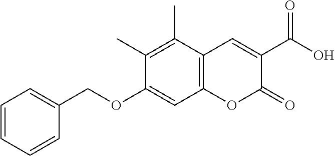 [SMILES: Cc1c(OCc2ccccc2)cc2oc(=O)c(C(=O)O)cc2c1C] | 7-(benzyloxy)-5,6-dimethyl- 2-oxo-2H-chromene-3- carboxylic acid |
| cpd 111 | 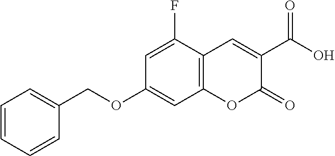 [SMILES: O=C(O)c1cc2c(F)cc(OCc3ccccc3)cc2oc1=O] | 7-(benzyloxy)-5-fluoro-2- oxo-2H-chromene-3- carboxylic acid |
| cpd 112 | 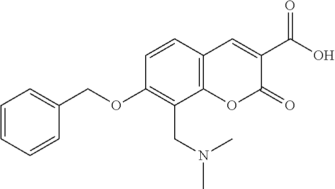 [SMILES: CN(C)Cc1c(OCc2ccccc2)ccc2cc(C(=O)O)c(=O)oc12] | 7-(benzyloxy)-8- ((dimethylamino)methyl)-2- oxo-2H-chromene-3- carboxylic acid |
| cpd 113 | 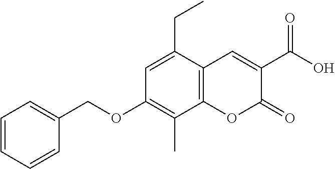 [SMILES: CCc1cc(OCc2ccccc2)c(C)c2oc(=O)c(C(=O)O)cc12] | 7-(benzyloxy)-5-ethyl-8- methyl-2-oxo-2H-chromene- 3-carboxylic acid |
| cpd 114 | 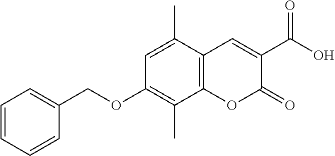 [SMILES: Cc1cc(OCc2ccccc2)c(C)c2oc(=O)c(C(=O)O)cc12] | 7-(benzyloxy)-5,8-dimethyl- 2-oxo-2H-chromene-3- carboxylic acid |
| cpd 115 | 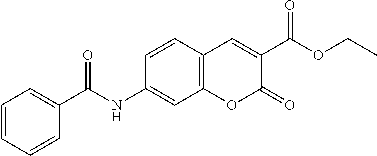 [SMILES: CCOC(=O)c1cc2ccc(NC(=O)c3ccccc3)cc2oc1=O] | ethyl 7-benzamido-2-oxo- 2H-chromene-3-carboxylate |
| cpd 116 | 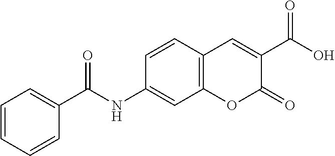 [SMILES: O=C(Nc1ccc2cc(C(=O)O)c(=O)oc2c1)c1ccccc1] | 7-benzamido-2-oxo-2H- chromene-3-carboxylic acid |

### Part A

The LC/MS analysis mentioned in the experimental part were performed on a Dionex Ultimate 3000 HPLC system (equipped with a PDA detector) connected to a mass spectrometer Brucker Esquire 6000 (equipped with a multimode source, ESI/APCI).

The separations were performed with a SunFireC18, 3.5 μm 3.0×100 mm, column equipped with a SunFire C18, 3.5 μm, 3.0×20 mm Guard column or a X-Bridge C18 100×3.0 mm column equipped with a X-Bridge 018, 3.5 μm, 3.0×20 mm Guard column thermostated to 30° C. and the DAD acquisition wavelength was set in the range of 190-420 nm.

Elutions were carried out with the methods described in the following tables

| LC/MS | Time | Solvents | | | Flow | |
|---|---|---|---|---|---|---|
| Method | (min) | A (%) | B (%) | C (%) | (mL/min) | Column |
| L1 | 0 | 80 | — | 20 | 1 | SunFire |
| | 0.2 | 80 | — | 20 | 1 | C18 |
| | 7 | 40 | — | 60 | 1 | |
| | 8 | 10 | — | 90 | 1 | |
| | 10.8 | 10 | — | 90 | 1 | |
| | 11 | 80 | — | 20 | 1 | |
| | 14 | 80 | — | 20 | 1 | |
| L2 | 0 | 50 | — | 50 | 1 | SunFire |
| | 0.2 | 50 | — | 50 | 1 | C18 |
| | 6 | 10 | — | 90 | 1 | |
| | 10.8 | 10 | — | 90 | 1 | |
| | 11 | 50 | — | 50 | 1 | |
| | 14 | 50 | — | 50 | 1 | |
| L3 | 0 | — | 80 | 20 | 1 | X-Bridge |
| | 0.2 | — | 80 | 20 | 1 | C18 |
| | 7 | — | 40 | 60 | 1 | |
| | 8 | — | 10 | 90 | 1 | |
| | 10.8 | — | 10 | 90 | 1 | |
| | 11 | — | 80 | 20 | 1 | |
| | 14 | — | 80 | 20 | 1 | |
| L4 | 0 | — | 50 | 50 | 1 | X-Bridge |
| | 0.2 | — | 50 | 50 | 1 | C18 |
| | 6 | — | 10 | 90 | 1 | |
| | 10.8 | — | 10 | 90 | 1 | |
| | 11 | — | 50 | 50 | 1 | |
| | 14 | — | 50 | 50 | 1 | |

Solvent A:: Formic Acid LC-MS grade 0.1% in milliQ water
Solvent B: NH4OAc (LC-MS grade) 10 mMol in milliQ water, adjusted at pH 10 with an aqueous solution of NH3, LC-Ms grade
Solvent C: Acetonitrile LC-MS grade

All the preparative HPLC purifications mentioned in this experimental part have been carried out with the following system: a Waters 2489 UV/Visible Detector, a Waters 2545 Binary Gradient Module, a Waters Fraction Collector III and a Waters Dual Flex Injector.

The separations were performed with a X-Bridge Prep C18 column, 100×19 mm, 5 μm column equipped with a X-Bridge C18, 19×10 mm, 5 μm Guard column or with a SunFire Prep C18 ODB column (5 μm; 19×100 mm) equipped with a SunFire C18 guard column (5 μm; 19×10 mm).

Elutions were carried out with the methods described in the following tables, and detection wavelengths were fixed at 210 and 254 nm.

| HPLC | Time | Solvent | | Flow | |
|---|---|---|---|---|---|
| Method | (min) | A (%) | B (%) | (mL/min) | Column |
| H1 | 0 | 80 | 20 | 20 | X-Bridge |
| | 2 | 80 | 20 | 20 | Prep C18 |
| | 8 | 10 | 90 | 20 | |
| | 10.8 | 10 | 90 | 20 | |
| | 11 | 80 | 20 | 20 | |
| | 16 | 80 | 20 | 20 | |
| H2 | 0 | 95 | 5 | 20 | SunFire |
| | 2 | 95 | 5 | 20 | Prep C18 |
| | 8 | 50 | 50 | 20 | ODB |
| | 9 | 10 | 90 | 20 | |
| | 13 | 10 | 90 | 20 | |
| | 14 | 95 | 5 | 20 | |
| | 16 | 95 | 5 | 20 | |

Solvent A: Ammonium Acetate puriss p.a. for HPLC 10 mM in milliQ water, adjusted at pH 10 with Ammonium Hydroxide puriss p.a. for HPLC
Solvent B: Acetonitrile HPLC grade.

### Example 1

### Synthesis of 7-methoxy-2-oxo-2H-chromene-3-carboxylic acid (cpd 2)

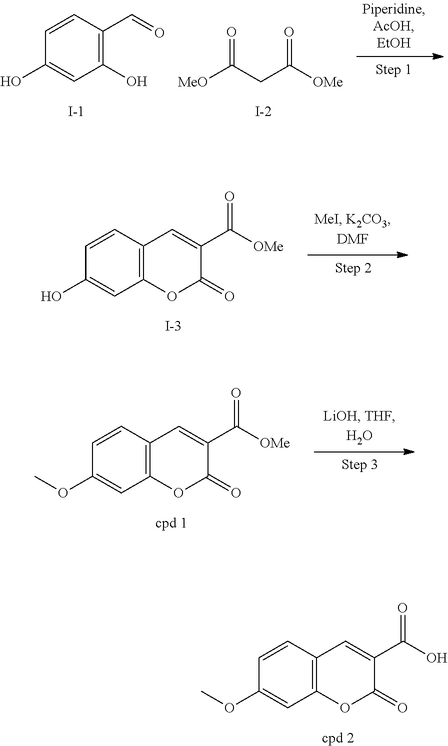

Step 1: Dimethyl malonate (I-2) (1.2 eq) was slowly added to a solution of 2,4-dihydroxybenzaldehyde (I-1) (1.0 eq) in ethanol. A catalytic amount of piperidine and acetic acid were added dropwise to the reaction. The reaction mixture was stirred and heated under reflux 3 h. After cooling, the resulting precipitate was filtered to afford methyl 7-hydroxy-2-oxo-2H-chromene-3-carboxylate (I-3). The solid was used in the next step without more purification. ¹H NMR (300 MHz, DMSO): δ ppm 8.71 (s, 1H), 7.76 (d, 1H), 6.85 (d, 1H), 6.74 (s, 1H), 3.80 (s, 3H). ESI/APCI(+): 221.1 (M+H). ESI/APCI(−): 218.9 (M−H).

Step 2: Methyl iodide (1.1 eq.) and potassium (or cesium) carbonate (1.0 eq.) were added to a solution methyl 7-hydroxy-2-oxo-2H-chromene-3-carboxylate (I-3) (1.0 eq) in DMF (3.5 ml per mmol of I-3). The reaction mixture was refluxed overnight. After cooling, an equivalent volume of distilled water was added to the DMF solution before liquid extraction with ethyl acetate. The organic layer was washed with a saturated solution of LiSO₄. The final organic layer was dried over Na₂SO₄, filtered and concentrated under reduced pressure. The resulting powder was recrystallized in EtOH to afford methyl 7-methoxy-2-oxo-2H-chromene-3-carboxylate (cpd 1). ¹H NMR (300 MHz, DMSO): δ ppm 8.76 (s, 1H), 7.86 (d, 1H), 7.04-7.01 (m, 2H), 3.90 (s, 3H), 3.81 (s, 3H). ESI/APCI(+): 235.1 (M+H).

Step 3: Lithium hydroxide (10 eq.) was added to a solution of 7-methoxy-2-oxo-2H-chromene-3-carboxylate (cpd 1) in a mixture of THF/water (1/1). The reaction mixture was refluxed during 1 hour. After cooling, the mixture was concentrated under reduced pressure. The resulting precipitate was solubilized in a minimum volume of an aqueous saturated solution of NH₄OH and Water in a ratio of ¼. A solution of HCl (36%) was carefully added dropwise to the stirred solution until precipitation at low acidic pH. The resulting precipitate was filtered and recrystallized in EtOH to afford 7-methoxy-2-oxo-2H-chromene-3-carboxylic acid (cpd 2) with a yield of 56.6%. ¹H NMR (300 MHz, DMSO): δ ppm 8.69 (s, 1H), 7.80 (d, 1H), 7.00 (s, 1H), 6.98 (d, 1H), 3.88 (s, 3H). APCI(+): 221.09 (M+H)⁺.

### Example 2-11

### Synthesis of Compounds Cpd 3, Cpd 5, Cpd 7, Cpd 9, Cpd 10, Cpd 11, Cpd 13, Cpd 15, Cpd 17 and Cpd 19

Following the procedure described above for Example 1 and substituting the appropriate reagents and starting materials and purification methods known to those skilled in the art, the following compounds of the present invention were prepared.

| | Reactionnel | Global | APCI (+) | |
|---|---|---|---|---|
| Code | Sequence | Yield (%) | (M + H): | ¹H NMR |
| cpd 3 | step | 43.2 | 433.01 | (300 MHz, DMSO): δ ppm 13.04 (s, 1H), 8.73 (s, 1H), 8.22 |
| | [1, 2 and 3] | | | (s, 2H), 8.13 (s, 1H), 7.87 (d, 1H), 7.18 (s, 1H), 7.15 (d, |
| | | | | 1H), 5.45 (s, 2H). |
| cpd 5 | step | 19.7 | 347.09 | (300 MHz, DMSO): δ ppm 8.24 (s, 1H), 8.10 (d, 1H), 7.97 |
| | [1, 2 and 3] | | | (dd, 2H), 7.73-7.51 (m, 5H), 7.21 (s, 1H), 7.05 (d, 1H), 5.67 |
| | | | | (s, 2H). |
| cpd 7 | step | 45 | 314.84 | (300 MHz, DMSO): δ ppm 13.02 (s, 1H), 8.73 (s, 1H), 7.85 |
| | [1, 2 and 3] | | | (d, 1H), 7.47 (dd, 1H), 7.34 (s, 1H), 7.33 (d, 1H), 7.23-7.09 |
| | | | | (m, 3H), 5.29 (s, 2H). |
| cpd 9 | step | 31.8 | 296.93 | (300 MHz, DMSO): δ ppm 8.72 (s, 1H), 7.84 (d, 1H), 7.50- |
| | [1, 2 and 3] | | | 7.36 (m, 5H), 7.14 (s, 1H), 7.09 (d, 1H), 5.26 (s, 2H). |
| cpd 10 | step | — | 387.1 | (300 MHz, DMSO): δ ppm 8.78 (s, 1H), 7.88 (d, 1H), 7.50- |
| | [1 and 2] | | | 7.39 (m, 10H), 7.14 (s, 1H), 7.08 (d, 1H), 5.32 (s, 2H), 5.26 |
| | | | | (s, 2H). |
| cpd 11 | step | 57.3 | 331.03 | (300 MHz, DMSO): δ ppm 13.02 (s, 1H), 8.73 (s, 1H), 7.85 |
| | [1, 2 and 3] | | | (d, 1H), 7.65-7.40 (m, 4H), 7.17 (d, 1H), 7.09 (dd, 1H), |
| | | | | 5.29 (s, 2H). |
| cpd 13 | step | 40.3 | 327.08 | (300 MHz, DMSO): δ ppm 13.01 (s, 1H), 8.71 (s, 1H), 7.83 |
| | [1, 2 and 3] | | | (d, 1H), 7.32 (t, 1H), 7.11-7.04 (m, 4H), 6.91 (d, 1H), 5.22 |
| | | | | (s, 2H), 3.75 (s, 3H). |
| cpd 15 | step | 9.6 | 310.96 | (300 MHz, DMSO): δ ppm 8.70 (s, 1H), 7.79 (d, 1H), 7.33- |
| | [1, 2 and 3] | | | 7.22 (m, 5H), 7.02 (s, 1H), 6.98 (d, 1H), 4.34 (t, 2H), 3.07 |
| | | | | (t, 2H). |
| cpd 17 | step | 13.9 | 343.16 | (300 MHz, DMSO): δ ppm 12.98 (s, 1H), 8.72 (s, 1H), 7.81 |
| | [1, 2 and 3] | | | (d, 1H), 7.01 (s, 1H), 6.98 (d, 1H), 5.44 (t, 1H), 5.04 (t, |
| | | | | 1H), 4.68 (d, 2H), 2.09 (s, 4H), 1.72 (s, 3H), 1.59 (s, 3H), |
| | | | | 1.55 (s, 3H). |
| cpd 19 | step | 32.8 | 362.98 | (300 MHz, DMSO): δ ppm 13.01 (s, 1H), 8.72 (s, 1H), 7.84 |
| | [1, 2 and 3] | | | (d, 1H), 7.77 (s, 1H), 7.68 (d, 1H), 7.47 (d, 1H), 7.13-7.07 |
| | | | | (m, 2H), 5.27 (s, 2H). |

### Example 12

### Synthesis of 7-((2,5-dimethoxybenzyl)oxy)-2-oxo-2H-chromene-3-carboxylic acid (cpd 22)

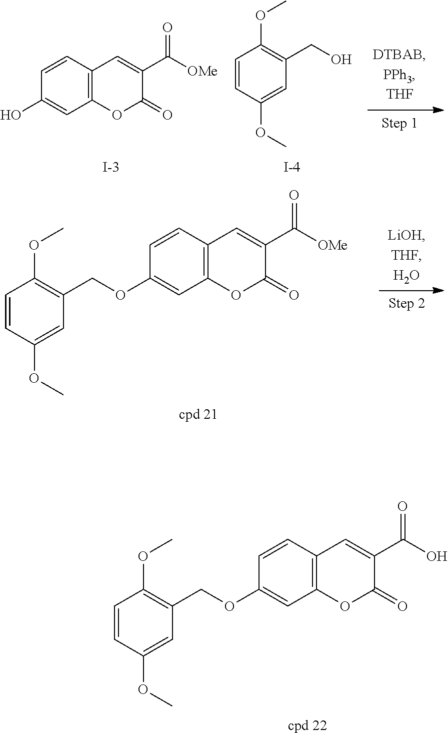

Step 1: DTBAD (1.2 eq.) and triphenylphosphine (1.2 eq) were added to a solution of (2,5-dimethoxyphenyl)methanol (1-4) (1.0 eq.) and methyl 7-hydroxy-2-oxo-2H-chromene-3-carboxylate (1-3) (1.0 eq) dissolved in THF (8 ml per mmol of 1-3). The reaction mixture was stirred overnight at room temperature. An aqueous solution of HCl (1 N) was added to the reaction mixture. The organic layer was extracted with dichloromethane, dried over Na₂SO₄ and concentrated under reduced pressure. The resulting powder was recrystallized in EtOH to afford methyl 7-((2,5-dimethoxybenzyl)oxy)-2-oxo-2H-chromene-3-carboxylate (cpd 21).

Step 2: Lithium hydroxide (10 eq.) was added to a solution of methyl 7-((2,5-dimethoxybenzyl)oxy)-2-oxo-2H-chromene-3-carboxylate (cpd 21) in a mixture of THF/water (1/1). The reaction mixture was refluxed during 1 hour. After cooling, the mixture was concentrated under reduced pressure. The resulting precipitate was solubilized in a minimum volume of an aqueous saturated solution of NH₄OH and Water in a ratio of ¼. A solution of HCl (36%) was carefully added dropwise to the stirred solution until precipitation at low acidic pH. The resulting precipitate was filtered and recrystallized in EtOH to afford 7-((2,5-dimethoxybenzyl)oxy)-2-oxo-2H-chromene-3-carboxylic acid (cpd 22) with a yield of 19.9%. ¹H NMR (300 MHz, DMSO): δ ppm 13.00 (s, 1H), 8.72 (s, 1H), 7.83 (d, 1H), 7.11-6.92 (m, 5H), 5.15 (s, 2H), 3.77 (s, 3H), 3.69 (s, 3H). APCI(+): 357.09 (M+H)⁺.

### Example 13-16

### Synthesis of Cpd 24, Cpd 26, Cpd 28 and Cpd 30

Following the procedure described above for Example 12 and substituting the appropriate reagents and starting materials and purification methods known to those skilled in the art, the following compounds of the present invention were prepared.

| | Global | APCI (+) | |
|---|---|---|---|
| Code | Yield (%) | (M + H): | ¹H NMR |
| cpd 24 | 21.3 | 337.97 | (300 MHz, DMSO): δ ppm 13.11 (s, 1H), 8.72 (s, 1H), 8.13 (d, |
| | | | 1H), 8.03 (d, 1H), 7.87 (d, 1H), 7.67 (dd, 1H), 7.49 (dd, 1H), |
| | | | 7.38 (s, 1H), 7.18 (d, 1H), 6.98 (s, 2H). |
| cpd 26 | 19.9 | 315.28 | (300 MHz, DMSO): δ ppm 13.02 (s, 1H), 8.72 (s, 1H), 7.84 (d |
| | | | 1H), 7.56 (d, 1H), 7.53 (d, 1H), 7.27-7.06 (m, 4H), 5.23 (s, 2H). |
| cpd 28 | 55.1 | 365.06 | (300 MHz, DMSO): δ ppm 13.02 (s, 1H), 8.72 (s, 1H), 7.87- |
| | | | 7.68 (m, 5H), 7.13-7.09 (m, 2H), 5.38 (s, 2H). |
| cpd 30 | 17.7 | 323.2 | (300 MHz, DMSO): δ ppm 13.01 (s, 1H), 8.71 (s, 1H), 7.81 (d, |
| | | | 1H), 7.27-7.15 (m, 4H), 7.06 (s, 1H), 6.96 (d, 1H), 5.39 (s, 1H), |
| | | | 3.43 (d, 2H), 3.05 (d, 2H). |

### Example 17

### Synthesis of 7-(benzyl(methyl)amino)-2-oxo-2H-chromene-3-carboxylic acid (cpd 31)

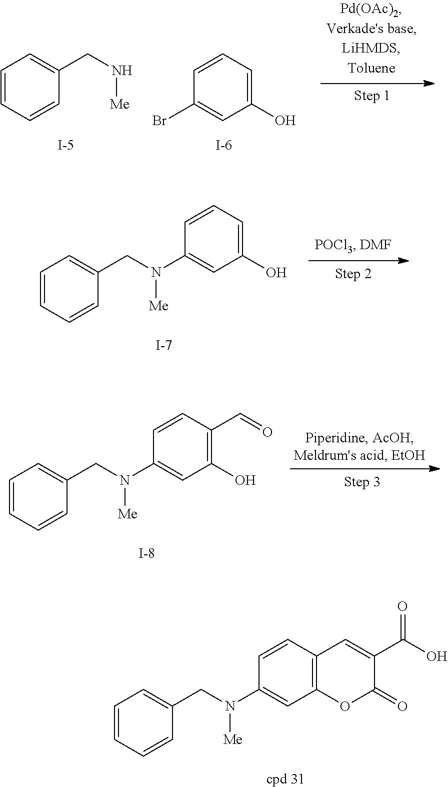

Step 1: N-Methyl-1-phenylmethanamine (1-5) (1.2 eq.) and 3-bromophenol (1-6) (1.0 eq) were stirred together under argon. After 10 min., Pd(OAc)₂ (2 mol %) was added to the reaction mixture. Verkade's base (0.0063 mol %) was then added dropwise to the reaction mixture. LiHMDS (2.3 eq., 1M in THF) was carefully added, followed by freshly distilled toluene (3.5 ml for 1 mmol of 1-6). The reaction mixture was refluxed during 24 h. After cooling, the mixture was extracted with a toluene/water solution, the organic phase was finally dried over Na₂SO₄, filtrated and concentrated under reduced pressure. The residue was purified by chromatography on silica gel using ethyl acetate (20%) in heptane to afford 3-(benzyl(methyl)amino)phenol (1-7).

Step 2: Phosphoryl chloride POCl₃ (1.2 eq. in 3.0 eq. of anhydrous DMF) was carefully added dropwise to a solution of 3-(benzyl(methyl)amino)phenol (1-7) in anhydrous DMF (200 μl for 1 mmol of 1-7) under argon at 0° C. The reaction mixture was stirred 15 min. at 0° C., then 15 min. at room temperature, 15 min. at 37° C. and finally 30 min. at 80-90° C. After cooling, ice and Na₂CO₃ were added to the reaction mixture. The precipitate was filtered off to afford 4-(benzyl(methyl)amino)-2-hydroxybenzaldehyde (1-8). The solid was used in the next step without more purification

Step 3: Meldrum's acid (1.2 eq.) was added to a solution of 4-(benzyl(methyl)amino)-2-hydroxybenzaldehyde (I-8) (1.0 eq.) dissolved in EtOH (10 ml for 1 mmol of I-8). to the stirred solution. A catalytic amount of piperidine and acetic acid were added dropwise to the reaction. The solution was stirred and heated 3 h under reflux. After cooling, the yellow to orange precipitate was filtered to afford 7-(benzyl(methyl)amino)-2-oxo-2H-chromene-3-carboxylic acid (cpd 31) with a yield of 12.2%. ¹H NMR (300 MHz, DMSO): δ ppm 8.59 (s, 1H), 7.64 (d, 1H), 7.35-7.20 (m, 5H), 6.86 (d, 1H), 6.62 (s, 1H), 4.78 (s, 2H), 3.20 (s, 3H). APCI(+): 309.98 (M+H).

### Example 18-23

### Synthesis of Cpd 32, Cpd 33, Cpd 34, Cpd 35, Cpd 36 and Cpd 37

Following the procedure described above for Example 17 and substituting the appropriate reagents and starting materials and purification methods known to those skilled in the art, the following compounds of the present invention were prepared.

| | Reactionnel | Global | APCI (+) | |
|---|---|---|---|---|
| Code | Sequence | Yield (%) | (M + H): | ¹H NMR |
| cpd 32 | step | 11.6 | 273.97 | (300 MHz, DMSO): δ ppm 8.04 (s, 1H), 7.48 (d, H), |
| | [1, 2 and 3] | | | 6.92 (d, 1H), 6.72 (s, 1H), 3.34 (s, 6H), 1.59 (s, 6H). |
| cpd 33 | step | 22.1 | 259.97 | (300 MHz, DMSO): δ ppm 8.58 (s, 1H), 7.64 (d, 1H), |
| | [1, 2 and 3] | | | 6.66 (d, 1H), 6.43 (s, 1H), 3.39 (s, 4H), 1.99 (s, 4H). |
| cpd 34 | step | 18.9 | 275.93 | (300 MHz, DMSO): δ ppm 8.59 (s, 1H), 7.68 (d, 1H), |
| | [2 and 3] | | | 7.01 (d, 1H), 6.83 (s, 1H), 3.72 (t, 4H), 3.41 (t, 4H). |
| cpd 35 | step | 22.7 | 233.93 | (300 MHz, DMSO): δ ppm 8.60 (s, 1H), 7.66 (d, 1H), |
| | [2 and 3] | | | 6.81 (d, 1H), 6.58 (s, 1H), 3.09 (s, 6H). |
| cpd 36 | step [3] | 59 | 261.91 | (300 MHz, DMSO): δ ppm 12.52 (s, 1H), 8.58 (s, 1H), |
| | | | | 7.63 (d, 1H), 6.78 (d, 1H), 6.56 (s, 1H), 3.48 (d,, |
| | | | | 4H), 1.14 (m, 6H). |
| cpd 37 | step [3] | 24 | 285.95 | (300 MHz, DMSO): δ ppm 8.44 (s, 1H), 7.22 (s, 1H), |
| | | | | 3.35 (s, 4H), 2.71 (d, 4H), 1.88 (d, 4H). |

### Example 24

### Synthesis of Cpd 38, Cpd 40 and 7-(benzyl(ethyl)amino)-2-oxo-2H-chromene-3-carboxylic acid (Cpd 41)

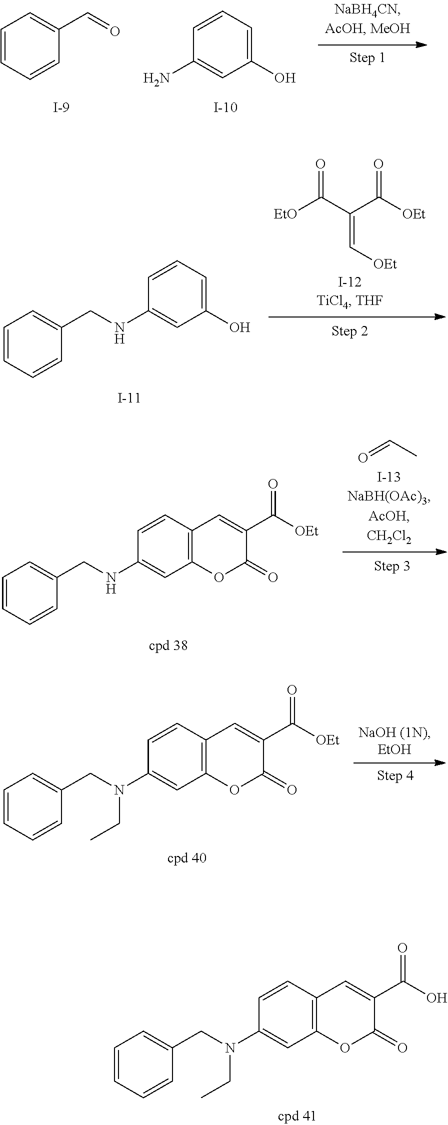

Step 1: A solution of 3-aminophenol (1-10) (1 eq.), benzaldehyde (1-9) (1 eq.); and glacial acetic acid (1 eq.) in methanol (7 mL per mmol of 1-10) was stirred under an argon atmosphere for 3 hours. Sodium cyanoborohydride (2 eq.) was added by portion and the reaction mixture was stirred for 3 hours at room temperature, after which the solvent was removed under reduced pressure. The residue was dissolved in ethyl acetate, and the solution was washed with water and brine, dried over magnesium sulfate, and concentrated under reduced pressure. The residue was purified by flash chromatography on silica gel using a gradient of ethyl acetate (2 to 10%) in dichloromethane to afford 91% of 3-(benzylamino)phenol (I-11) an oily residue. ESI/APCI(+): 200 (M+H).

Step 2: Titanium(IV) chloride (1.25 eq.) was added to a stirred solution of 3-(benzylamino)phenol (I-11) (1.0 eq.) and diethyl 2-(ethoxymethylene)malonate (1-12) (1.1 eq.) in THF (1.5 mL per 1 mmol of I-11). The mixture was stirred at 85° C. for 20 hours. The cooled reaction mixture was poured with stirring into water (17 mL per 1 mmol of I-11). The water insoluble product was collected and purified by flash chromatography on silica gel using a gradient of ethyl acetate (2 to 10%) in dichloromethane to afford 26% of ethyl 7-(benzylamino)-2-oxo-2H-chromene-3-carboxylate (cpd 38) as a yellow solid. ¹H NMR (300 MHz, DMSO): δ ppm 8.52 (s, 1H), 7.90 (t, 1H), 7.55 (d, 1H), 7.37 (d, 4H), 7.22-7.32 (m, 1H), 6.71 (d, 1H), 6.42 (s, 1H), 4.43 (d, 2H), 4.22 (q, 2H), 1.27 (t, 3H). ESI/APCI(+): 324 (M+H), 346 (M+Na). ESI/APCI(−): 322 (M−H).

Step 3: Acetic acid (1.1 eq.) was added to a solution of ethyl 7-(benzylamino)-2-oxo-2H-chromene-3-carboxylate (cpd 38) (1 eq.) and acetaldehyde (1-13) (10 eq.) in dry dichloromethane (4 mL per 1 mmol of cpd 38). The mixture was stirred at room temperature for 5 hours and then sodium triacetoxyborohydride (1.1 eq.) was added. The mixture was stirred at room temperature for 40 hours and was then diluted with dichloromethane and quenched with an aqueous solution of sodium bicarbonate. After separation, the organic layer was concentrated under reduced pressure and the residue was purified by flash chromatography on silica gel using a gradient of ethyl acetate (1 to 10%) in dichloromethane to afford 91% of ethyl 7-(benzyl(ethyl)amino)-2-oxo-2H-chromene-3-carboxylate (cpd 40) as an oily residue. ESI/APCI(+): 352 (M+H), 374 (M+Na).

Step 4: Ethyl 7-(benzyl(ethyl)amino)-2-oxo-2H-chromene-3-carboxylate (cpd 40) (1 eq.) was stirred in a mixture of ethanol (4.6 mL per mmol of cpd 40) and an aqueous solution of sodium hydroxide 1M (4.6 eq.). After 3 hours, the reaction mixture was acidified to pH 3 by addition of a solution of hydrochloric acid 1 M. The formed precipitate was collected by filtration and dried to give 69% of 7-(benzyl(ethyl)amino)-2-oxo-2H-chromene-3-carboxylic acid as a yellow solid. ¹H NMR (300 MHz, DMSO): δ ppm 12.57 (br. s, 1H), 8.58 (s, 1H), 7.63 (d, 1H), 7.18-7.40 (m, 5H), 6.82 (d, 1H), 6.59 (br. s., 1H), 4.74 (s, 2H), 3.63 (q, 2H), 1.18 (t, 3H). ESI/APCI(+): 324 (M+H), 306 (M+H−H₂O), 346 (M+Na). ESI/APCI(−): 278 (M−H—CO₂).

### Example 25 and 26

### Synthesis of Compounds Cpd 39 and Cpd 44

Following the procedure described above for Example 24 and substituting the appropriate reagents and starting materials and purification methods known to those skilled in the art, the following compounds of the present invention were prepared.

| | | | LC/MS | | | | | |
|---|---|---|---|---|---|---|---|---|
| | Reac- | Global | | | Retention | | | |
| | tionnel | Yield | APCI (+) | LC/MS | Time | Purity | Purification | |
| Code | Sequence | (%) | (M + H)+: | Condition | (min.) | (%) | Type | Conditions |
| cpd 39 | Step | 7 | 296 | L2 | 2.1 | 99.2 | Flash | gradient |
| | [1, 2 and 4] | | | | | | chroma- | of MeOH |
| | | | | | | | tography | (0-6%) in |
| | | | | | | | on silica | CH₂Cl₂ |
| | | | | | | | gel | |
| cpd 44 | Step | 3.7 | 338 | L2 | 4.0 | 100 | HPLC | H1 |
| | [1, 2, 3 and 4] | | | | | | | |

### Example 27

### Synthesis of Cpd 42 and Cpd 43

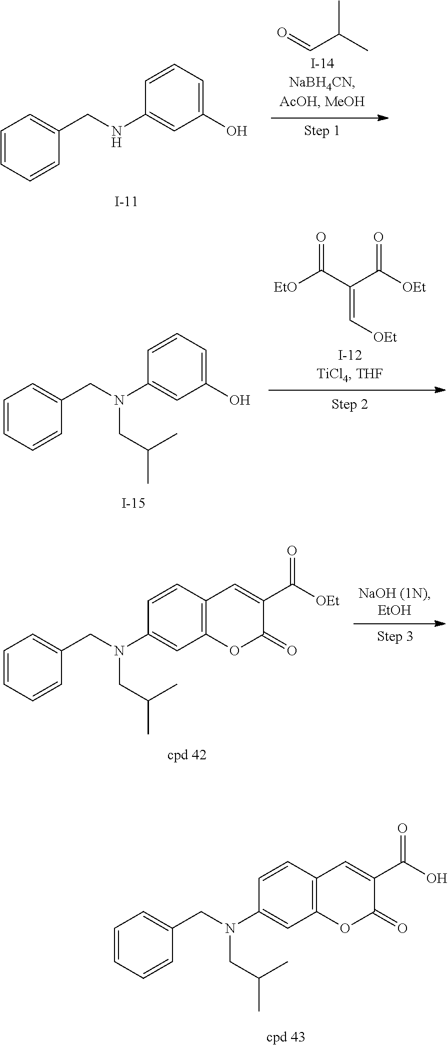

Step 1: A solution of 3-(benzylamino)phenol (I-11) (1 eq.), isobutyraldehyde (1-14) (1.2 eq.); and glacial acetic acid (1 eq.) in methanol (7 mL per mmol of 1-10) was stirred under an argon atmosphere for 3 hours. Sodium cyanoborohydride (2 eq.) was added by portion and the reaction mixture was stirred for 72 hours at room temperature, after which the solvent was removed under reduced pressure. The residue was dissolved in ethyl acetate, and the solution was washed with water and brine, dried over magnesium sulfate, and concentrated under reduced pressure. The residue was purified by flash chromatography on silica gel using a gradient of ethyl acetate (1 to 6%) in dichloromethane to afford 23% of 3-(benzyl(isobutyl)amino)phenol (1-15) as an oily residue. ESI/APCI(+): 256 (M+H).

Step 2: Titanium(IV) chloride (1.25 eq.) was added to a stirred solution of 3-(benzyl(isobutyl)amino)phenol (1-15) (1.0 eq.) and diethyl 2-(ethoxymethylene)malonate (I-12) (1.1 eq.) in THF (1.1 mL per 1 mmol of I-15). The mixture was stirred at 85° C. for 20 hours. The cooled reaction mixture partitioned between dichloromethane and water. After separation, the organic layer was concentrated under reduced pressure and the residue was purified by flash chromatography on silica gel using a gradient of ethyl acetate (0 to 6%) in dichloromethane to afford 60% of ethyl 7-(benzyl(isobutyl)amino)-2-oxo-2H-chromene-3-carboxylate (cpd 42) as a yellow an oily residue. ESI/APCI(+): 380 (M+H).

Step 3: Ethyl 7-(benzyl(isobutyl)amino)-2-oxo-2H-chromene-3-carboxylate (cpd 42) (1 eq.) was stirred in a mixture of ethanol (6.6 mL per mmol of cpd 42) and an aqueous solution of sodium hydroxide 1M (3.4 eq.). After 3 hours, the reaction mixture was acidified to pH 3 by addition of a solution of sodium bisulfate acid 1 M. The formed precipitate was collected by filtration purified by flash chromatography on silica gel using a gradient of methanol (0 to 3%) in dichloromethane to give 17% of 7-(benzyl(isobutyl)amino)-2-oxo-2H-chromene-3-carboxylic acid as a yellow solid. ESI/APCI(+): 352 (M+H).

### Example 28-34

### Synthesis of Compounds Cod 45, Cod 46, Cod 47, Cod 48, Cod 49, Cpd 50 and Cpd 51

Following the procedure described above for Example 27 and substituting the appropriate reagents and starting materials and purification methods known to those skilled in the art, the following compounds of the present invention were prepared.

| | | Global | | LC/MS | | | |
|---|---|---|---|---|---|---|---|
| | Reactionnel | Yield | | APCI (+) | Con- | Retention | Purity |
| Code | Sequence | (%) | ¹H NMR | (M + H)+: | dition | Time (min.) | (%) |
| cpd 45 | Step | 61 | (300 MHz, CDCl₃) δ ppm: 12.32 (br. s., 1H), | 276 | L2 | 2.8 | 96.9 |
| | [1, 2 and 3] | | 8.65 (s, 1H), 7.45 (d, 1H), 6.74 (d, 1H), 6.53 | | | | |
| | | | (br. s., 1H), 3.30 (d, 2H), 3.16 (s, 3H), 1.91- | | | | |
| | | | 2.30 (m, 1H), 0.98 (d, 6H) | | | | |
| cpd 46 | Step | 5 | (300 MHz, CDCl₃) δ ppm: 12.31 (br. s., 1H), | 316 | L2 | 2.3 | 97.4 |
| | [1, 2 and 3] | | 8.71 (s, 1H), 7.51 (d, 1H), 7.21-7.25 (m, 1H), | | | | |
| | | | 6.97 (d, 2H), 6.86 (d, 1H), 6.68 (br. s., 1H), | | | | |
| | | | 4.84 (s, 2H), 3.23 (s, 3H) | | | | |
| cpd 47 | Step | 10 | (300 MHz, CDCl₃) δ ppm: 12.24 (br. s., 1H), | 328 | L2 | 2.8 | 99.4 |
| | [1, 2 and 3] | | 8.70 (s, 1H), 7.49 (d, 1H), 7.29-7.40 (m, 1H), | | | | |
| | | | 6.93-7.05 (m, 2H), 6.82 (dd, 2H), 6.60 (br. s., | | | | |
| | | | 1H), 4.71 (s, 2H), 3.26 (s, 3H) | | | | |
| cpd 48 | Step | 30 | (300 MHz, CDCl₃) δ ppm: 12.31 (br. s., 1H), | 316 | L2 | 4.5 | 99.3 |
| | [1, 2 and 3] | | 8.67 (s, 1H), 7.46 (d, 1H), 6.73 (d, 1H), 6.53 | | | | |
| | | | (br. s., 1H), 3.31 (d, 2H), 3.14 (s, 3H), 1.51- | | | | |
| | | | 1.89 (m, 6H), 1.15-1.35 (m, 3H), 0.88-1.15 | | | | |
| | | | (m, 2H) | | | | |
| cpd 49 | Step | 12 | (300 MHz, CDCl₃) δ ppm: 8.68 (s, 1H), 7.46 (d, | 340 | L2 | 2.6 | 96.8 |
| | [1, 2 and 3] | | J = 9.0 Hz, 1H), 7.27-7.35 (m, 1H), 6.66- | | | | |
| | | | 6.86 (m, 4H), 6.60 (br. s., 1H), 4.68 (s, 2H), | | | | |
| | | | 3.79 (s, 3H), 3.25 (s, 3H) | | | | |
| cpd 50 | Step | 16 | (300 MHz, CDCl₃) δ ppm: 12.32 (s, 1H), 8.69 (s, | 318 | L1 | 5.2 | 95.2 |
| | [1, 2 and 3] | | 1H), 7.49 (d, 1H), 6.74 (d, 1H), 6.55 (br. s., | | | | |
| | | | 1H), 4.01 (d, 2H), 3.27-3.44 (m, 4H), 3.17 (s, | | | | |
| | | | 3H), 1.95-2.11 (m, 1H), 1.57-1.64 (m, 2H), | | | | |
| | | | 1.33-1.53 (m, 2H) | | | | |
| cpd 51 | Step | 5 | (300 MHz, CDCl₃) δ ppm: 12.4 (br. s., 1H), 8.68 | 386 | L2 | 4.3 | 90.3 |
| | [1, 2 and 3] | | (s, 1H), 7.31-7.48 (m, 7H), 7.13-7.25 (m, | | | | |
| | | | 4H), 6.82 (d, 8.3 Hz, 1H), 6.66 (br. s., 1H), | | | | |
| | | | 4.80 (s., 4H) | | | | |

### Example 35

### Synthesis of Cpd 115 and 7-benzamido-2-oxo-2H-chromene-3-carboxylic acid (Cpd 116)

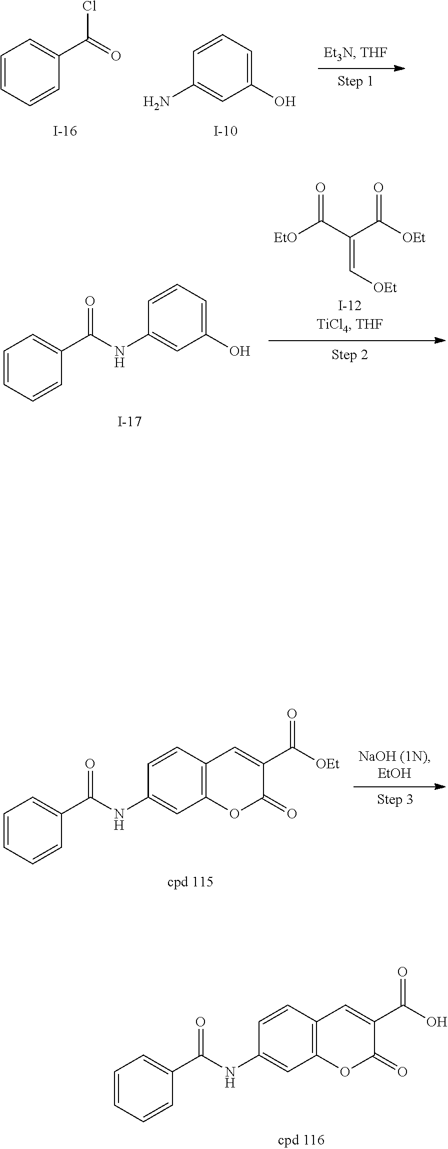

Step 1: Benzoyl chloride (1-16) (12.7 mL; 109.9 mmol) was added to a solution of 3-aminophenol (1-10) (4 g; 36.65 mmol), triethylamine (20.4 mL; 146.6 mL) in THF (200 mL). The mixture was stirred overnight at room temperature, then it was diluted with water. HCl (6N) was added till pH 1 and the aqueous phase was extracted with EtOAc (2×). The combined organic layers were dried over magnesium sulfate, and concentrated under reduced pressure. The residue was dissolved in THF/water (4/1; 250 mL) and LiOH (6.3 g; 146.6 mmol) was added. After 3 h, the reaction mixture was acidified till pH 1-2 and aqueous phase was extracted with EtOAc (2×). The combined organic phases were washed with a saturated solution of sodium hydrogen carbonate and brine. The organic layer was dried over magnesium sulfate and filtered. The solvent was removed under reduced pressure to furnish 7.64 (97%) of the desired compound (1-17) as a white solid. ESI/APCI(−): 212 (M−H).

Step 2: Titanium(IV) chloride (1.9 mL; 11.8 mmol) was added to a stirred solution of N-(3-hydroxyphenyl)benzamide (2 g; 9.4 mmol) and diethyl 2-(ethoxymethylene)malonate (2.1 mL; 10.3 mmol) (1-17) in THF (10 mL). The mixture was stirred at 85° C. for 20 hours in a sealed tube. The cooled reaction mixture was poured with stirring into water (30 mL). The water insoluble product was filtered off and purified by flash chromatography on silica gel using a gradient of ethyl acetate (0 to 10%) in dichloromethane to afford 0.2 g (6%) of the desired product (cpd 115) as a white solid. ESI/APCI(+): 338 (M+H). ESI/APCI(−): 336 (M−H).

Step 3: Ethyl 7-benzamido-2-oxo-2H-chromene-3-carboxylate (cpd 115) (0.07 g; 0.2 mmol) was stirred in a mixture of ethanol (1.5 mL) and an aqueous solution of sodium hydroxide (1M; 1 mL; 1 mmol). After 3 hours, the reaction mixture was acidified to pH 3 by addition of a solution of hydrochloric acid (1 M). The formed precipitate was collected by filtration and dried to give 0.042 g (65%) of the desired product (cpd 116) as a yellow solid. ESI/APCI(+): 310 (M+H). ESI/APCI(−): 308 (M−H). In the LCMS condition L1, retention time was 6.4 minutes and purity was 95.4%.

### Part B

### Example 36

### Evaluation of the Inhibition of Lactate Influx in SiHa Cells Via [¹⁴C]-Lactate Uptake Assay

SiHa cells were seeded on flat-bottom 24-well plates previously coated with poly-L-lysine (500 000 cells/well). After 6 h of incubation, the culture medium was replaced by 1 ml of DMEM containing 10 mM lactate and incubated overnight for metabolic adaptation. Cells were first rinsed with a modified Krebs solution (containing 10 μM L-lactate, without glucose) and pre-exposed to vehicle or increasing concentrations of compounds of the invention (0.1, 1, 10 and 100 μM) at 37° C. in the modified Krebs solution before addition of 2 μM [¹⁴C]-lactate for 12 min. Cells were then rinsed with an ice-cold D-lactate-containing Krebs solution (10 μM D-Lactate, without glucose) and lysed with 0.1 M NaOH. Sample aliquots were then incubated with liquid scintillation solution (Microscint 40) into a 96-well plate (Optiplate). After a 1 h agitation, radioactivity was measured (PerkinElmer Topcount); cpm values were normalized per protein amounts.

Representative compounds listed in table 1 have an IC₅₀ between 10 nM and 100 μM when tested in lactate influx assay described in the methodology herein above. Examples of the activity of compounds of the invention as inhibitors of the lactate influx are shown in table 2.

TABLE 2

| | ¹⁴C-Lactate uptake |
|---|---|
| Code | IC₅₀ (μM) |
| cpd 31 | 0.011 |
| cpd 36 | 0.25 |

### Example 37

### Evaluation of the Inhibition of Lactate Influx in SiHa Cells Via Lactate Consumption Assay

SiHa cells were seeded on flat-bottom 24-well plates (500 000 cells/well) in normal DMEM. After 6 h of incubation, the culture medium was replaced by 1 ml of DMEM containing 10 mM lactate and incubated overnight for metabolic adaptation. Cells were then treated for 24 h with increasing concentrations of compounds (0.1, 1, 10 and 100 μM) in lactate-containing medium at 37° C. The supernatants were then centrifuged using deproteinizing columns (15 min, 10000 g at 4° C.) and lactate concentration was determined using the enzymatic assay commercialized by CMA Microdialysis AB on a CMA600 analyzer (Aurora Borealis).

Representative compounds listed in table 1 have an IC₅₀ between 50 nM and 100 μM when tested in lactate influx assay described in the methodology herein above. Examples of the activity of compounds of the invention as inhibitors of the lactate influx are shown in table 3.

TABLE 3

| | Lactate |
|---|---|
| | consumption assay |
| Code | IC₅₀ (μM) |
| cpd 2 | 2.3 |
| cpd 9 | 1.9 |
| cpd 15 | 2.4 |
| cpd 26 | 1.25 |
| cpd 31 | 0.059 |
| cpd 34 | 7.9 |
| cpd 35 | 1.4 |
| cpd 36 | 0.86 |
| cpd 37 | 1.3 |

### Example 38

### Cytotoxicity Assay to Evaluate the Cytotoxic and/or Anti-Proliferative Activity of the Compounds of the Invention on Cancer Cells Using Lactate as Energy Source

SiHa cells (human cervix squamous carcinoma) were routinely cultured in DMEM containing serum and antibiotics as previously described in Sonveaux, P. et al. J. Clin. Invest 2008 118, 3930. Two distinct media were used for the cytotoxicity assay, either DMEM containing 25 mM D-glucose (without pyruvate) or DMEM containing 10 mM L-lactate (without glucose). SiHa cells were seeded in flat-bottom 96-well plates in normal DMEM. After 6 h of incubation, the culture medium was replaced by 100 μl of glucose- or lactate-containing medium and incubated overnight for metabolic adaptation. SiHa cells were treated with the compounds of the invention at a concentration range of 0.01 μM to 100 μM. After a 72 h incubation at 37° C., cell medium was removed and replaced by freshly prepared MTT in PBS (1 mg/ml, 100 μl/well). After a 3 h incubation at 37° C., the plates were centrifuged (1000 g, 10 min at 4° C.) and the supernatant was removed before addition of DMSO (100 μl/well). Plates were kept for 10 min in the dark before reading at the spectrophotometer (Victor X4).

Representative compounds listed in table 1 have a CC₅₀ between 40 nM and 100 μM when tested in the cytotoxicity and/or antiproliferative assay using lactate as energy source as described in the methodology herein above. Representative compounds of the invention were not cytotoxic when using glucose in the medium as described above. Examples of the activity of compounds of the invention are shown in table 4.

TABLE 4

| | Lactate cytotoxicity assay |
|---|---|
| Code | CC₅₀ (μM) |
| cpd 2 | 9.8 |
| cpd 3 | 0.6 |
| cpd 5 | 1.09 |
| cpd 7 | 1.17 |
| cpd 9 | 9.1 |
| cpd 11 | 4.23 |
| cpd 13 | 4.6 |
| cpd 15 | 5.6 |
| cpd 17 | 11.7 |
| cpd 19 | 68.4 |
| cpd 22 | 0.702 |
| cpd 24 | 1.1 |
| cpd 26 | 3.9 |
| cpd 28 | 6.86 |
| cpd 30 | 7.8 |
| cpd 31 | 0.22 |
| cpd 33 | 14.3 |
| cpd 34 | 17.5 |
| cpd 35 | 2.9 |
| cpd 36 | 1.7 |
| cpd 37 | 1.2 |
| cpd 39 | 1.79 |
| cpd 41 | 1.9 |
| cpd 43 | 16.3 |
| cpd 44 | 11.1 |
| cpd 45 | 0.22 |
| cpd 46 | 0.21 |
| cpd 47 | 0.04 |
| cpd 48 | 0.2 |
| cpd 49 | 0.07 |
| cpd 50 | 0.1 |
| cpd 51 | 12.7 |
| cpd 116 | 1.5 |

### Example 39

### In Vivo Evaluation of the Anti-Cancer Activity of the Compounds of the Invention Against Cervix Cancer

7 weeks old female nude mice (Janvier®, 4-5 mice per group, Balb-c or NMRI) with various human cancer cell lines xenografts were used. SiHa cells were subcutaneously injected (10⁶ to 2.10⁶ (10⁶ or 2.10⁶) cells in sterile NaCl 0.9% per mouse) on the left flank of the mice (100 μl/mouse). The treatment of the mice with compounds of the invention was started when tumor xenografts reach approximately 5 mm of length and width (Caliper® measurement). cpd 31 and cpd 36 (3 mg/kg) were daily injected intraperitoneally (IP 50 μl in DMSO). Tumor volume was calculated as following: (length×width²×π)/6 and expressed in mm³. Tumor growth delay graphs and statistical analysis (Two-way ANOVA) were performed using GraphPad Prism 4 software.

Both compounds tested (cpd 31 and cpd 36) showed a clear tumor growth delay as shown in FIGS. 1(A) and 1(B).

### Example 40

### In Vivo Evaluation of the Anti-Cancer Activity of the Compounds of the Invention Against Colon Cancer

Seven weeks old female nude mice (Janvier®, 4-5 mice per group, NMRI), were used for the experiments. HCT116 cells were subcutaneously injected (2.10⁶ cells in sterile NaCl 0.9% per mouse) on the left flank of the mice (100 μl/mouse). The treatment of the mice with compounds of the invention was started when tumor xenografts reached approximately 5 mm of length and width (Caliper® measurement). CPD 36 and Cpd 31(3 mg/kg) were daily injected intraperitoneally (IP 50 μl in DMSO). Tumor volume was calculated as the following: (length×width²×π)/6 and expressed in mm³. Tumor growth delay graphs and statistical analysis (Two-way ANOVA) were performed using GraphPad Prism 4 software.

Both compounds tested (cpd 31 and cpd 36) showed a clear tumor growth delay as shown in FIG. 2.

### Example 41

### In Vivo Evaluation of the Anti-Cancer Activity of the Compounds of the Invention in Combination with Chemotherapy Against Cervix Cancer

Seven weeks old female nude mice (Janvier®, 4-5 mice per group, NMRI), were used for the experiments. SiHa cells were subcutaneously injected (2.10⁶ cells in sterile NaCl 0.9% per mouse) on the left flank of the mice (100 μl/mouse). The treatment of the mice with compounds of the invention was started when tumor xenografts reached approximately 5 mm of length and width (Caliper® measurement). Cisplatin was injected IP at day 0 and day 7 (5 mg/kg) with/without cpd 31 or cpd 36 which were injected from day 1-6 and 8-11 (3 mg/kg daily injected intraperitoneally 50 μl in DMSO). Tumor volume was calculated as the following: (length×width²×π)/6 and expressed in mm³. Tumor growth delay graphs and statistical analysis (Two-way ANOVA) were performed using GraphPad Prism 4 software.

Both compounds tested (cpd 31 and cpd 36) showed a clear delay in tumor relapse as shown in FIG. 3.

1. A compound according to formula (A) for use in the prevention and/or treatment of cancer in a subject, 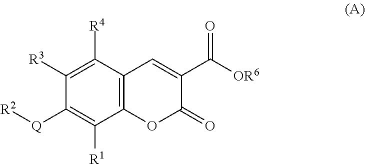
wherein,
each R¹, R³ and R⁴ is independently selected from hydrogen; halogen; hydroxyl; sulfhydryl; trifluoromethyl; trifluoromethoxy; nitro; amino; cyano; alkyl; alkenyl; alkynyl; heteroalkyl; heteroalkenyl; and heteroalkynyl;
wherein said alkyl, alkenyl, alkynyl, heteroalkyl, heteroalkenyl, and heteroalkynyl can be unsubstituted or substituted with one or more substituents selected from R⁷; or
R¹ can be combined with R² to form a 6, or 7 membered heterocycle; or R³ can be combined with R² to form a 5, 6, or 7 membered heterocycle; or wherein R¹ or R³ can be combined with R⁵ to form a 5, 6, or 7 membered heterocycle;
Q is independently selected from 0 and NR⁵;
R² is independently selected from alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; aryl heteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
R² is taken together with R⁵ to form a 5, 6, or 7 membered heterocycle which can be unsubstituted or substituted with one or more substituents selected from R⁸;
R⁵ is independently selected from hydrogen; alkyl; cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
and wherein said alkyl, cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
R⁵ is taken together with R² to form a 4, 5, 6, or 7 membered heterocycle which can be unsubstituted or substituted with one or more substituents selected from R⁸;
R⁶ is independently selected from hydrogen; and alkyl;
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
and isomers (in particular stereo-isomers or tautomers), solvates, salts (in particular pharmaceutically acceptable salts) or prodrugs thereof.

2. The compound of claim 1, wherein R⁶ is hydrogen.

3. The compound according to claim 1, wherein R¹, R³ and R⁴ are hydrogen.

4. The compound according to claim 1, wherein R⁵ is selected from hydrogen and C₁₋₉ alkyl.

5. A compound of formula (D2) and isomers (in particular stereo-isomers or tautomers), solvates, salts (in particular pharmaceutically acceptable salts) or prodrugs thereof, 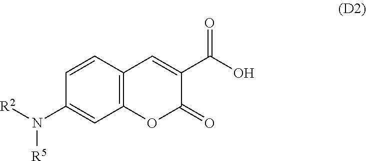
wherein,
R² is independently selected from cycloalkyl; alkenyl; cycloalkenyl; alkynyl; cycloalkynyl; heteroalkyl; heteroalkenyl; heteroalkynyl; aryl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
and wherein said cycloalkyl, alkenyl, cycloalkenyl, alkynyl, cycloalkynyl, heteroalkyl, heteroalkenyl, heteroalkynyl, aryl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl; heterocycle-heteroalkenyl, or heterocycle-heteroalkynyl can be unsubstituted or substituted with one or more substituents selected from R⁸; or
R⁵ is independently selected from alkyl; cycloalkyl; alkenyl; cycloalkenyl; alkynyl; cycloalkynyl; heteroalkyl; heteroalkenyl; heteroalkynyl; heterocycle; arylalkyl; arylalkenyl; arylalkynyl; arylheteroalkyl; arylheteroalkenyl; arylheteroalkynyl; heterocycle-alkyl; heterocycle-alkenyl; heterocycle-alkynyl; heterocycle-heteroalkyl, heterocycle-heteroalkenyl; or heterocycle-heteroalkynyl;
and wherein said alkyl, cycloalkyl, alkenyl, cycloalkenyl, alkynyl, cycloalkynyl, heteroalkyl, heteroalkenyl, heteroalkynyl, heterocycle, arylalkyl, arylalkenyl, arylalkynyl, arylheteroalkyl, arylheteroalkenyl, arylheteroalkynyl, heterocycle-alkyl, heterocycle-alkenyl, heterocycle-alkynyl, heterocycle-heteroalkyl, heterocycle-heteroalkenyl, or heterocycle-heteroalkynyl can be unsubstituted or substituted with one or more substituents selected from R⁹; or
R⁷ is independently selected from hydroxyl; ═O; halogen; —SH; ═S; trifluoromethyl; —OCF₃; cyano; nitro; —C(O)OH; and NH₂;
each R⁸ and R⁹ is independently selected from alkyl; alkenyl; alkynyl; heteroalkyl; heteroalkenyl; heteroalkynyl; hydroxyl; ═O; halogen; —SH; ═S; trifluoromethyl; —OCF₃; cyano; nitro; —C(O)OH; or NH₂;
and isomers (in particular stereo-isomers or tautomers), solvates, salts (in particular pharmaceutically acceptable salts) or prodrugs thereof.

6. The compound according to claim 5, wherein said compound is a compound of formula (E2), or (E4) and isomers (in particular stereo-isomers or tautomers), solvates, salts (in particular pharmaceutically acceptable salts) or prodrugs thereof, 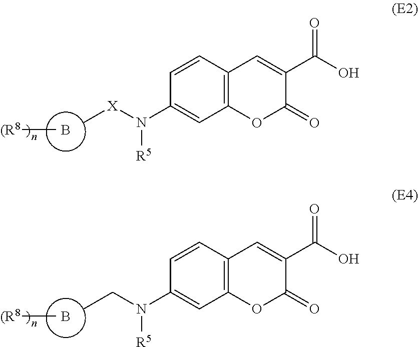
wherein,
each R⁵ and R⁸ is as in claim 5;
n is selected from 0; 1; 2; 3 and 4;
X is selected from alkylene, —CO—, —SO₂—, or represents a single bond (thereby establishing a direct bond between 0 and cycle B for formula (E1) or N and cycle B for formula (E2)); and
cycle B is selected from cycloalkyl; cycloalkenyl; cycloalkynyl; aryl; and heterocycle.

7. The compound according to claim 6, wherein
n is selected from 0; 1; 2; 3 and 4;
X is selected from alkylene, or represents a single bond (thereby establishing a direct bond between N and cycle B for formula (E2)); and cycle B is selected from cycloalkyl; cycloalkenyl; cycloalkynyl; aryl; and heterocycle.

8. The compound according to claim 6, wherein said compound is a compound of formula (E5), or (E6) and isomers (in particular stereo-isomers or tautomers), solvates, salts (in particular pharmaceutically acceptable salts) or prodrugs thereof, 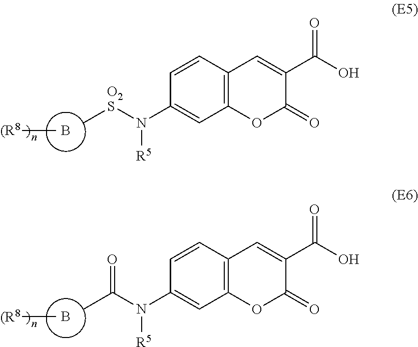
wherein, each of R⁸ and R⁵ have the same meaning as that defined in claim 6,
n is selected from 0; 1; 2; 3 and 4; and
cycle B is selected from cycloalkyl; cycloalkenyl; cycloalkynyl; aryl; and heterocycle.

9. The compound according to claim 5, for use in the prevention and/or treatment of cancer in a subject.

10. The compound according to claim 9, wherein the cancer is a solid cancer.

11. The compound according to claim 9, wherein the cancer is a cancer expressing MCT1 and/or MCT4.

12. The compound according to claim 10, wherein the cancer is a solid cancer expressing MCT1 and/or MCT4.

13. The compound according to claim 9, wherein the cancer is selected from cervix cancer and colon cancer.

14. A pharmaceutical composition comprising the compound according to claim 1 in combination with a pharmaceutically acceptable carrier.

15. A method for the prevention or treatment of a cancer in an animal, mammal or human comprising administering to said animal, mammal or human in need for such prevention or treatment an effective dose of the compound according to claim 1.
